SEQUENCE LISTING (19 sequences)

SEQ ID NO: 1 - protein, 8 aa, unidentified
GYTFTSYY

SEQ ID NO: 2 - protein, 8 aa, unidentified
INPSNAGT

SEQ ID NO: 3 - protein, 12 aa, unidentified
TREGNPYYTMNY

SEQ ID NO: 4 - protein, 12 aa, unidentified
QSLLNSGNQKTY

SEQ ID NO: 5 - skipped

SEQ ID NO: 6 - protein, 9 aa, unidentified
QNDHSYPFT

SEQ ID NO: 7 - protein, 119 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSS

SEQ ID NO: 8 - protein, 113 aa, unidentified
DIVMTQSPDSLAVSLGERATISCKSSQSLLNSGNQKTYLAWYQQKPGQPPKLLIYGASTRESGVPDRFSGSGSGTDFTLTISSLQAEDVAVYYCQNDHSYPFTFGQGTKLEIK

SEQ ID NO: 9 - protein, 449 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 220 aa, unidentified
DIVMTQSPDSLAVSLGERATISCKSSQSLLNSGNQKTYLAWYQQKPGQPPKLLIYGASTRESGVPDRFSGSGSGTDFTLTISSLQAEDVAVYYCQNDHSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 446 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 449 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 449 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 446 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 446 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 446 aa, unidentified
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 446 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 10 aa, unidentified
FLSRPTEKTI

(19) United States
(12) Patent Application Publication
(10) Pub. No.: US 2025/0059264 A1
(43) Pub. Date: Feb. 20, 2025

(54) METHOD OF TREATING STROKE

(71) Applicant: AlaMab Therapeutics, Inc., Princeton, NJ (US)

(72) Inventor: Yanfeng Zhang, Phoenixville, PA (US)

(21) Appl. No.: 18/803,013

(22) Filed: Aug. 13, 2024

Related U.S. Application Data
(60) Provisional application No. 63/519,465, filed on Aug. 14, 2023.

(51) Int. Cl. C07K 16/18; A61P 9/10

(52) U.S. Cl. CPC C07K 16/18; A61P 9/10; C07K 2317/24

(57) ABSTRACT

The present disclosure provides compositions and methods of treating a stroke in a subject. The method comprises blocking the opening of connexin 43 (Cx43) hemichannel in cells by, e.g., using a composition comprising an anti-Cx43 antibody.

### CROSS-REFERENCE TO RELATED APPLICATION

This application claims priority to U.S. Provisional Application Ser. No. 63/519,465, filed on Aug. 14, 2023, the disclosure of which is hereby incorporated by reference in its entirety.

### INCORPORATION OF SEQUENCE LISTING

The instant application contains a Sequence Listing, which has been submitted via Patent Center. The Sequence Listing titled 172628-203003_US.xml, which was created on Aug. 2, 2024, and is 20,916 bytes in size, is hereby incorporated by reference in its entirety.

### BACKGROUND

Stroke is a leading cause of death and disability worldwide. It can cause permanent neurologic damage or even death if not promptly diagnosed and treated. It is the third leading cause of death and the leading cause of adult disability in the United States and industrialized European nations.

During a stroke, blood perfusion into the brain is disturbed by an occlusion or hemorrhage of blood vessels, with part of the brain with disturbed perfusion no longer receives adequate oxygen (hypoxia). The lack of adequate oxygen causes cells in the brain to die or be seriously damaged, impairing brain function. A stroke also results in a transient loss of blood-brain barrier function, which in turn leads to a loss of ionic and neurotransmitter homeostasis, influx of immune cells and water into the brain, and swelling of the brain. The death of brain tissue and the changes associated with the disruption of the blood-brain barrier following a stroke can severely impact neurological function and, in the case of severe stroke, even result in death. However, despite the medical emergency presented by stroke, options for treating stroke are limited.

Some cells in the brain express connexin Cx43 hemichannels, and these channels mediate the passage of small molecules (less than 1.2 kDa) between inside/outside of the cell. Hemichannels are normally closed, but under certain conditions, such as mechanical stress and inflammatory conditions, they are activated and opened. Opened Cx43 hemichannels in cells in the brain can promote the release of pro-inflammatory factors, such as prostaglandin E2 (PGE2) and ATP, and can contribute to the loss of ion and neurotransmitter homeostasis associated with a stroke. Inhibiting the opening of Cx43 hemichannels in cells (e.g., by chemical reagents, etc.) can ameliorate the death of brain tissue, swelling and disruption of neurological function associated with stroke in a subject.

Thus, there remains a need for therapies for effective treatment of stroke and other pathological conditions associated with increased hemichannel activity.

### SUMMARY

In one aspect, provided herein is a method for treating a stroke in a subject, comprising administering to the subject at least one dose of an anti-connexin 43 (Cx43) antibody, wherein the anti-Cx43 antibody comprises heavy chain CDR sequences and light chain CDR sequences as follows:
HCDR1: SEQ ID NO: 1;
HCDR2: SEQ ID NO: 2;
HCDR3: SEQ ID NO: 3;
LCDR1: SEQ ID NO: 4;
LCDR2: SEQ ID NO: 5; and
LCDR3: SEQ ID NO: 6.

In some embodiments, the stroke is an ischemic stroke. In some embodiments, wherein the stroke is a hemorrhagic stroke.

In some embodiments, the at least one dose of the anti-Cx43 antibody is about 0.01 mg/kg to about 100 mg/kg. In some embodiments, the at least one dose of the anti-Cx43 antibody is about 15 mg/kg. In some embodiments, the at least one dose of the anti-Cx43 antibody is about 25 mg/kg. In some embodiments, the at least one dose of the anti-Cx43 antibody is about 50 mg/kg.

In some embodiments, the method comprises administering a first dose of the anti-Cx43 antibody within 2 weeks from the stroke. In some embodiments, the method comprises administering a first dose of the anti-Cx43 antibody within one week from the stroke. In some embodiments, the method comprises administering a first dose of the anti-Cx43 antibody within 6 hours from the stroke. In some embodiments, the method comprises administering a first dose of the anti-Cx43 antibody within 2 hours from the stroke. In some embodiments, the method comprises administering a first dose of the anti-Cx43 antibody within 30 minutes from the stroke.

In some embodiments, the method comprises administering a second dose of the anti-Cx43 antibody at one day to four weeks after the first dose is administered. In some embodiments, the method comprises administering a second dose of the anti-Cx43 antibody at least four days after the first dose is administered. In some embodiments, the method comprises administering one or more subsequent doses of the anti-Cx43 antibody after the second dose is administered.

In some embodiments, the anti-Cx43 antibody is administered intravenously.

In some embodiments, at least post-stroke criterion is assessed within three to six months after the at least one dose of the anti-Cx43 antibody is administered. In some embodiments, at least post-stroke criterion is assessed at least one day to two weeks after the at least one dose of the anti-Cx43 antibody is administered. In some embodiments, at least post-stroke criterion is assessed at least one to two days after the at least one dose of the anti-Cx43 antibody is administered. In some embodiments, the at least one post-stroke criterion comprises one or more of: (a) neurological function, (b) cerebral infarct volume, and (c) cerebral edema. In some embodiments, the at least one post-stroke criterion is improved after the at least one dose of the anti-Cx43 antibody is administered.

In some embodiments, the anti-Cx43 antibody comprises heavy chain variable sequence of SEQ ID NO: 7, and/or light chain variable sequence of SEQ ID NO: 8. In some embodiments, the anti-Cx43 antibody comprises heavy chain sequence of any one of SEQ ID NOs: 9 and 11-18, and/or light chain variable sequence of SEQ ID NO: 10. In some embodiments, the anti-Cx43 antibody comprises heavy chain sequence of SEQ ID NO: 9, and/or light chain variable sequence of SEQ ID NO: 10.

In another aspect, provided herein is a method for treating a stroke in a subject, comprising administering to the subject an effective amount of an anti-connexin 43 (Cx43) antibody, wherein the anti-Cx43 antibody is administered according to the following dosing regimen: i) a first dose within one week from the stroke, and ii) a second dose within a month from the stroke.

In some embodiments, the method comprises administering a first dose of the anti-Cx43 antibody within 6 hours from the stroke. In some embodiments, the method comprises administering a first dose of the anti-Cx43 antibody within 2 hours from the stroke. In some embodiments, the method comprises administering a first dose of the anti-Cx43 antibody within 30 minutes from the stroke. In some embodiments, the method comprises administering a second dose of the anti-Cx43 antibody at least four days after the first dose is administered. In some embodiments, the method further comprises administering one or more subsequent doses of the anti-Cx43 antibody after the second dose is administered.

In some embodiments, the first, second and/or subsequent dose of the anti-Cx43 antibody is about 0.01 mg/kg to about 100 mg/kg. In some embodiments, the first, second and/or subsequent dose of the anti-Cx43 antibody is about 15 mg/kg. In some embodiments, the first, second and/or subsequent dose of the anti-Cx43 antibody is about 25 mg/kg. In some embodiments, the first, second and/or subsequent dose of the anti-Cx43 antibody is about 50 mg/kg.

In some embodiments, the anti-Cx43 antibody is administered intravenously.

In some embodiments, the stroke is an ischemic stroke. In some embodiments, the stroke is a hemorrhagic stroke.

In some embodiments, at least post-stroke criterion is assessed within three to six months after the first, second and/or one or more subsequent doses of the anti-Cx43 antibody is administered. In some embodiments, at least post-stroke criterion is assessed at least one day to two weeks after first, second and/or one or more subsequent doses of the anti-Cx43 antibody is administered. In some embodiments, the at least one post-stroke criterion comprises one or more of: (a) neurological function, (b) cerebral infarct volume, and (c) cerebral edema. In some embodiments, at least one post-stroke criterion is improved after the first, second and/or one or more subsequent doses of the anti-Cx43 antibody is administered.

In some embodiments, the anti-Cx43 antibody comprises heavy chain CDR sequences and light chain CDR sequences as follows:
HCDR1: SEQ ID NO: 1;
HCDR2: SEQ ID NO: 2;
HCDR3: SEQ ID NO: 3;
LCDR1: SEQ ID NO: 4;
LCDR2: SEQ ID NO: 5; and
LCDR3: SEQ ID NO: 6.

In some embodiments, the anti-Cx43 antibody comprises heavy chain variable sequence of SEQ ID NO: 7, and/or light chain variable sequence of SEQ ID NO: 8. In some embodiments, the anti-Cx43 antibody comprises heavy chain sequence of any one of SEQ ID NOs: 9 and 11-18, and/or light chain variable sequence of SEQ ID NO: 10. In some embodiments, the anti-Cx43 antibody comprises heavy chain sequence of SEQ ID NO: 9, and/or light chain variable sequence of SEQ ID NO: 10.

In some embodiments of any of the methods provided herein, the subject is a human.

In some embodiments of any of the methods provided herein, the anti-Cx43 antibody blocks the opening of Cx43 hemichannel in the subject.

### BRIEF DESCRIPTION OF DRAWINGS

FIGS. 1A-1B depict the effect of treatment with an anti-Cx43 antibody on the beam walking time of a stroke mouse model. The stroke mouse model was established by medial cerebral artery occlusion (MCAO). The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 10 mg/kg. The beam walking time was measured 24 hours (FIG. 1A) and 48 hours (FIG. 1B) after infarction. Each data point corresponds to the mean beam walking time (±standard error of the mean [SEM], n=12). As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIGS. 2A-2B depict the effect of treatment with an anti-Cx43 antibody on the grip strength of a stroke mouse model. The stroke mouse model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 10 mg/kg. The grip strength was measured 24 hours (FIG. 2A) and 48 hours (FIG. 2B) after infarction. Each data point corresponds to the mean grip strength (±SEM, n=12). As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIGS. 3A-3B depict the effect of treatment with an anti-Cx43 antibody on the rotarod test score of a stroke mouse model. The stroke mouse model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 10 mg/kg. The rotarod test score was measured 24 hours (FIG. 3A) and 48 hours (FIG. 3B) after infarction. Each data point corresponds to the mean rotarod test score (±SEM, n=12). As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIGS. 4A-4G depict the effect of treatment with an anti-Cx43 antibody on the cerebral infarct volume of a stroke mouse model. The stroke mouse model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 10 mg/kg. At 48 hours after reperfusion, the whole brain tissue of the mice was harvested to harvested to perform 2,3,5-triphenyltetrazolium chloride (TTC) staining to measure the infarct area. FIG. 4A depicts the measurement of cerebral infarct volume. Each data point corresponds to the mean cerebral infarct volume (±SEM, n=6). FIGS. 4B-4G correspond to the TTC-stained brain slices for the sham (FIG. 4B), model (FIG. 4C), butylphthalide (FIG. 4D), and anti-Cx43 antibody 15 mg/kg, 25 mg/kg, and 50 mg/kg (FIG. 4E, FIG. 4F, and FIG. 4G, respectively) test groups used to assess the infarct area measurements shown in FIG. 4A. As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIG. 5 depicts the effect of treatment with an anti-Cx43 antibody on the brain water content of a stroke mouse model. The stroke mouse model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 10 mg/kg. At 48 hour after reperfusion, the whole brain tissue of the mice was harvested to measure the brain water content and calculate the ration of water content in the brain. Each data point corresponds to the mean brain water content (±SEM, n=6). As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIG. 6 depicts the effect of treatment with an anti-Cx43 antibody on the brain volume of a stroke mouse model. The stroke mouse model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 10 mg/kg. At 48 hours after reperfusion, the whole brain tissue of the mice was harvested to measure the brain volume. Each data point corresponds to the mean brain volume (±SEM, n=12). As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIGS. 7A-7B depict the effect of the time of treatment with an anti-Cx43 antibody on the beam walking time of a stroke mouse model. The stroke mouse model was established by medial cerebral artery occlusion (MCAO). The anti-Cx43 antibody was administered intravenously at a dose of 50 mg/kg at either 30 minutes, 2 hours, or 6 hours after the infarction. Butylphthalide was used as a control substance and was administered intravenously at a dose of 10 mg/kg at either 30 minutes, 2 hours, or 6 hours after the infarction. The beam walking time was measured 24 hours (FIG. 7A) and 48 hours (FIG. 7B) after infarction. Each data point corresponds to the mean beam walking time (±SEM, n=12). As used in the figure: sham, sham group (no stroke); Model, stroke mouse model (vehicle-treated); AM1, anti-Cx43 antibody ALMB-0166; ^{##}, p<0.01 vs. sham; and *, p<0.05 vs. model.

FIGS. 8A-8B depict the effect of the time of treatment with an anti-Cx43 antibody on the grip strength of a stroke mouse model. The stroke mouse model was established by MCAO. The anti-Cx43 antibody was administered intravenously at a dose of 50 mg/kg at either 30 minutes, 2 hours, or 6 hours after the infarction. Butylphthalide was used as a control substance and was administered intravenously at a dose of 10 mg/kg at either 30 minutes, 2 hours, or 6 hours after the infarction. The grip strength was measured 24 hours (FIG. 8A) and 48 hours (FIG. 8B) after infarction. Each data point corresponds to the mean grip strength (±SEM, n=12). As used in the figure: sham, sham group (no stroke); Model, stroke mouse model (vehicle-treated); AM1, anti-Cx43 antibody ALMB-0166; ^{##}, p<0.01 vs. sham; and *, p<0.05 vs. model.

FIGS. 9A-9B depict the effect of the time of treatment with an anti-Cx43 antibody on the rotarod test score of a stroke mouse model. The stroke mouse model was established by MCAO. The anti-Cx43 antibody was administered intravenously at a dose of 50 mg/kg at either 30 minutes, 2 hours, or 6 hours after the infarction. Butylphthalide was used as a control substance and was administered intravenously at a dose of 10 mg/kg at either 30 minutes, 2 hours, or 6 hours after the infarction. The rotarod test score was measured 24 hours (FIG. 9A) and 48 hours (FIG. 9B) after infarction. Each data point corresponds to the mean rotarod test score (±SEM, n=12). As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIGS. 10A-10G depicts the effect of the time of treatment with an anti-Cx43 antibody on the cerebral infarct volume of a stroke mouse model. The stroke mouse model was established by MCAO. The anti-Cx43 antibody was administered intravenously at a dose of 50 mg/kg at either 30 minutes, 2 hours, or 6 hours after the infarction. Butylphthalide was used as a control substance and was administered intravenously at a dose of 10 mg/kg at either 30 minutes, 2 hours, or 6 hours after the infarction. At 48 hours after reperfusion, the whole brain tissue of the mice was harvested to harvested to perform 2,3,5-triphenyltetrazolium chloride (TTC) staining to measure the infarct area. FIG. 10A depicts the measurement of cerebral infarct volume. Each data point corresponds to the mean cerebral infarct volume (±SEM, n=6). FIGS. 10B-10I correspond to the TTC-stained brain slices for the sham test group (FIG. 10B), the model test group (FIG. 10C), the test groups treated with butylphthalide at 30 minutes, 2 hours, or 6 hours post-infarction (FIG. 10D, FIG. 10E, and FIG. 10F, respectively), and the test groups treated with the anti-Cx43 antibody at 30 minutes, 2 hours, or 6 hours post-infarction (FIG. 10G, FIG. 10H, and FIG. 10I, respectively) corresponding to the infarct area measurements shown in FIG. 10A. As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIG. 11 depicts the effect of time of treatment with an anti-Cx43 antibody on the brain water content of a stroke mouse model. The stroke mouse model was established by MCAO. The anti-Cx43 antibody was administered intravenously at a dose of 50 mg/kg at either 30 minutes, 2 hours, or 6 hours after the infarction. Butylphthalide was used as a control substance and was administered intravenously at a dose of 10 mg/kg at either 30 minutes, 2 hours, or 6 hours after the infarction. At 48 hours after reperfusion, the whole brain tissue of the mice was harvested to measure the brain water content and calculate the ration of water content in the brain. Each data point corresponds to the mean brain water content (±SEM, n=6). As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIG. 12 depicts the effect of time of treatment with an anti-Cx43 antibody on the brain volume of a stroke mouse model. The stroke mouse model was established by MCAO. The anti-Cx43 antibody was administered intravenously at a dose of 50 mg/kg at either 30 minutes, 2 hours, or 6 hours after the infarction. Butylphthalide was used as a control substance and was administered intravenously at a dose of 10 mg/kg at either 30 minutes, 2 hours, or 6 hours after the infarction. At 48 hours after reperfusion, the whole brain tissue of the mice was harvested to measure the brain volume. Each data point corresponds to the mean brain volume (±SEM, n=12). As used in the figure: sham, sham group (no stroke); Model, stroke mouse model (vehicle-treated); AM1, anti-Cx43 antibody ALMB-0166; ^{##}, p<0.01 vs. sham; *, p<0.05 vs. model; and **, p<0.01 vs. model.

FIGS. 13A-13B depict the effect of treatment with an anti-Cx43 antibody on the beam walking time of a stroke rat model. The rat stroke model was established by medial cerebral artery occlusion (MCAO). The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 5 mg/kg. The beam walking time was measured 24 hours (FIG. 13A) and 48 hours (FIG. 13B) after infarction. Each data point corresponds to the mean beam walking time (±SEM, n=12). As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and **, p<0.01 vs. model.

FIGS. 14A-14B depict the effect of treatment with an anti-Cx43 antibody on the grip strength of a rat stroke model. The rat stroke model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 5 mg/kg. The grip strength was measured 24 hours (FIG. 2A) and 48 hours (FIG. 2B) after infarction. Each data point corresponds to the mean grip strength (±SEM, n=12). As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and **, p<0.01 vs. model.

FIGS. 15A-15B depict the effect of treatment with an anti-Cx43 antibody on the rotarod test score of a rat stroke model. The rat stroke model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 5 mg/kg. The rotarod test score was measured 24 hours (FIG. 15A) and 48 hours (FIG. 15B) after infarction. Each data point corresponds to the mean rotarod test score (±SEM, n=12). As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and **, p<0.01 vs. model.

FIGS. 16A-16G depicts the effect of treatment with an anti-Cx43 antibody on the cerebral infarct volume of a rat stroke model. The rat stroke model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 5 mg/kg. At 48 hour after reperfusion, the whole brain tissue of the rats was harvested to harvested to perform 2,3,5-triphenyltetrazolium chloride (TTC) staining to measure the infarct area. FIG. 16A depicts the measurement of cerebral infarct volume. Each data point corresponds to the mean cerebral infarct volume (±SEM, n=6). FIGS. 16B-16G correspond to the TTC-stained brain slices for the sham (FIG. 16B), model (FIG. 16C), butylphthalide (FIG. 16D), and anti-Cx43 antibody 15 mg/kg, 25 mg/kg, and 50 mg/kg (FIG. 16E, FIG. 16F, and FIG. 16G, respectively) test groups used to assess the infarct area measurements shown in FIG. 16A. As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and ** p<0.01 vs. model.

FIG. 17 depicts the effect of treatment with an anti-Cx43 antibody on the brain water content of a rat stroke model. The rat stroke model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 5 mg/kg. At 48 hours after reperfusion, the whole brain tissue of the rats was harvested to measure the brain water content and calculate the ration of water content in the brain. Each data point corresponds to the mean brain water content (±SEM, n=6). As used in the figure: sham, sham group (no stroke); Model, rat stroke model (vehicle-treated); AM1, anti-Cx43 antibody ALMB-0166; *, p<0.01 vs. sham; *, p<0.05 vs. model; and **, p<0.01 vs. model.

FIG. 18 depicts the effect of treatment with an anti-Cx43 antibody on the brain volume of a rat stroke model. The rat stroke model was established by MCAO. The anti-Cx43 antibody was administered intravenously 30 minutes after the infarction. Three different dosages of the anti-Cx43 antibody were tested (15 mg/kg, 25 mg/kg, and 50 mg/kg). Butylphthalide was used as a control substance and was administered intravenously, twice a day, at a dose of 5 mg/kg. At 48 hours after reperfusion, the whole brain tissue of the rats was harvested to measure the brain volume. Each data point corresponds to the mean brain volume (±SEM, n=12). As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and **, p<0.01 vs. model.

FIG. 19 depicts the effect of an anti-Cx43 antibody dosing regimen on the body weight of a stroke rat model. The rat stroke model was established by medial cerebral artery occlusion (MCAO). For anti-Cx43 antibody treatment, rats were intravenously administered a first dose at 30 minutes after the infarction and a second dose at Day 4 after the infarction. Three different dosages of the anti-Cx43 antibody were tested (15 mg/kg, 25 mg/kg, and 50 mg/kg). For butylphthalide treatment, rats were intravenously administered a first dose of 5 mg/kg butylphthalide at 30 minutes after the infarction and a second dose at Day 4 after the infarction. The body weight was measured at before MCAO (Day 0 of study) and at Days 2, 4, 7 and 11 after infarction. Each data point corresponds to the mean body weight (±SEM, n=6). As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and **, p<0.01 vs. model.

FIGS. 20A-20F depict the effect an anti-Cx43 antibody dosing regimen on the beam walking time of a stroke rat model. The rat stroke model was established by medial cerebral artery occlusion (MCAO). For anti-Cx43 antibody treatment, rats were intravenously administered a first dose at 30 minutes after the infarction and a second dose at Day 4 after the infarction. Three different dosages of the anti-Cx43 antibody were tested (15 mg/kg, 25 mg/kg, and 50 mg/kg). For butylphthalide treatment, rats were intravenously administered a first dose of 5 mg/kg butylphthalide at 30 minutes after the infarction and a second dose at Day 4 after the infarction. The beam walking time was measured at Day 1 (FIG. 20B), Day 2 (FIG. 20C), Day 4 (FIG. 20D), Day 7 (FIG. 20E) and Day 14 (FIG. 20F) after infarction. Each data point corresponds to the mean beam walking time (±SEM, n=6). As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and **, p<0.01 vs. model.

FIGS. 21A-21F depict the effect of an anti-Cx43 antibody dosing regimen on the grip strength of a rat stroke model. The rat stroke model was established by MCAO. For anti-Cx43 antibody treatment, rats were intravenously administered a first dose at 30 minutes after the infarction and a second dose at Day 4 after the infarction. Three different dosages of the anti-Cx43 antibody were tested (15 mg/kg, 25 mg/kg, and 50 mg/kg). For butylphthalide treatment, rats were intravenously administered a first dose of 5 mg/kg butylphthalide at 30 minutes after the infarction and a second dose at Day 4 after the infarction. The grip strength was measured at Day 1 (FIG. 21B), Day 2 (FIG. 21C), Day 4 (FIG. 21D), Day 7 (FIG. 21E) and Day 14 (FIG. 21F) after infarction. Each data point corresponds to the mean grip strength (±SEM, n=12). As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and **, p<0.01 vs. model.

FIGS. 22A-22F depict the effect of an anti-Cx43 antibody dosing regimen on the rotarod test score of a rat stroke model. The rat stroke model was established by MCAO. For anti-Cx43 antibody treatment, rats were intravenously administered a first dose at 30 minutes after the infarction and a second dose at Day 4 after the infarction. Three different dosages of the anti-Cx43 antibody were tested (15 mg/kg, 25 mg/kg, and 50 mg/kg). For butylphthalide treatment, rats were intravenously administered a first dose of 5 mg/kg butylphthalide at 30 minutes after the infarction and a second dose at Day 4 after the infarction. The rotarod test score was measured at Day 1 (FIG. 22B), Day 2 (FIG. 22C), Day 4 (FIG. 22D), Day 7 (FIG. 22E) and Day 14 (FIG. 22F) after infarction. Each data point corresponds to the mean rotarod test score (±SEM, n=12). As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and **, p<0.01 vs. model.

FIGS. 23A-23G depict the effect an anti-Cx43 antibody dosing regimen on the cerebral infarct volume of a rat stroke model. The rat stroke model was established by MCAO. For anti-Cx43 antibody treatment, rats were intravenously administered a first dose at 30 minutes after the infarction and a second dose at Day 4 after the infarction. Three different dosages of the anti-Cx43 antibody were tested (15 mg/kg, 25 mg/kg, and 50 mg/kg). For butylphthalide treatment, rats were intravenously administered a first dose of 5 mg/kg butylphthalide at 30 minutes after the infarction and a second dose at Day 4 after the infarction. At 48 h after reperfusion, the whole brain tissue of the rats was harvested to harvested to perform 2,3,5-triphenyltetrazolium chloride (TTC) staining to measure the infarct area. FIG. 23A depicts the measurement of cerebral infarct volume. Each data point corresponds to the mean cerebral infarct volume (±SEM, n=6). FIGS. 23B-23G correspond to the TTC-stained brain slices for the sham (FIG. 23B), model (FIG. 23C), butylphthalide (FIG. 23D), and anti-Cx43 antibody 15 mg/kg, 25 mg/kg, and 50 mg/kg (FIG. 23E, FIG. 23F, and FIG. 23G, respectively) test groups used to assess the infarct area measurements shown in FIG. 16A. As used in the figure: sham, sham group (no stroke); Model, rat stroke model (vehicle-treated); AM1, anti-Cx43 antibody ALMB-0166; ^{##}, p<0.01 vs. sham; *, p<0.05 vs. model; and **, p<0.01 vs. model.

### DETAILED DESCRIPTION

The present disclosure provides methods and compositions for treating stroke in a subject or patent in need thereof, comprising administering at least one dose of an anti-Cx43 antibody to the subject or patent. In some embodiments, the anti-Cx43 antibody blocks or inhibits the opening of Cx43 hemichannel. In some embodiments, the anti-Cx43 antibody comprises specific CDR amino acid sequences. In some embodiments, the anti-Cx43 antibody is administered according to a dosing regimen.

### I. Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “of”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% of a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “providing” is used according to its ordinary meaning to supply or furnish for use. In some embodiments, the protein (e.g., an antibody) is provided directly by administering the protein, while in other embodiments, the protein (e.g., an antibody) is effectively provided by administering a nucleic acid that encodes the protein. In certain aspects the invention contemplates compositions comprising various combinations of nucleic acid, antigens, peptides, and/or epitopes.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “percent sequence identity” or “sequence identity” refers to the degree of identity between any given query sequence and a subject sequence. A percent identity for any query nucleic acid or amino acid sequence, relative to another subject nucleic acid or amino acid sequence can be determined using tools and technologies known in the art, for example, NCBI BLAST.

The term “pharmaceutical formulation” refers to a preparation that contains a therapeutic agent (e.g., an anti-Cx43 antibody). In such form as to permit the biological activity of the antibody to be effective, and which contains no additional components which are unacceptably toxic to a subject to which the formulation would be administered.

As used herein, the term “antigen” is a molecule capable of being bound by an antibody or T-cell receptor. In certain embodiments, binding moieties other than antibodies are engineered to specifically bind to an antigen, e.g., aptamers, avimers, and the like.

As used herein, the term “specifically binds” is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some embodiments, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹. Antibodies may, for example, bind with an EC so of 50 nM or less, 10 nM or less, 1 nM or less, 100 pM or less, or more preferably 10 pM or less. As kwon in the art, a variety of immunoassay formats may be used to select antibodies specifically immunoreactive with an antigen. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

An “affinity matured” antibody has one or more alterations in one or more hypervariable regions thereof which result an improvement in the affinity of the antibody for antigen, compared to a parent antibody which does not possess those alteration(s). In one aspect, affinity matured antibodies will have nanomolar or even picomolar affinities for the target antigen. Affinity matured antibodies are produced by procedures known in the art. Marks et al., Bio Technology, 10:779-783 (1992) describes affinity maturation by VH and VL domain shuffling. Random mutagenesis of CDR and/or framework residues is described by: Barbas et al., Proc Nat. Acad. Sci. USA, 91:3809-3813 (1994); Schier et al., Gene, 169: 147-155 (1995); Yelton et al., J. Immunol., 155:1994-2004 (1995); Jackson et al., J. Immunol., 154(7):3310-9 (1995); and Hawkins et al., J. Mol. Biol., 226:889-896 (1992).

As used herein, “stroke” is a general term that refers to conditions caused by the occlusion or hemorrhage of one or more blood vessels supplying the brain, leading to cell death. A stroke can be an ischemic stroke or a hemorrhagic stroke. “Ischemic stroke”, as used herein, refers to stroke caused by an occlusion of one or more blood vessels supplying the brain. Types of ischemic stroke include, but are not limited to, embolic stroke, cardioembolic stroke, thrombotic stroke, large vessel thrombosis, lacunar infarction, artery-artery stroke and cryptogenic stroke. “Hemorrhagic stroke”, as used herein, refers to stroke caused by hemorrhage of one or more blood vessels supplying the brain. Types of hemorrhagic stroke include, but are not limited to, subdural stroke, intraparenchymal stroke, epidural stroke, and subarachnoid stroke.

As used herein, a “transient ischemic attack” or “mini-stroke” is an ischemic event that less than 24 hours. The transient ischemic attack is associated with the same symptoms as an ischemic stroke such as sudden numbness or weakness of the face, arm, or leg sudden confusion, trouble speaking or understanding, sudden trouble seeing in one or both eyes, and/or sudden trouble walking, dizziness, loss of balance or coordination. Typically, Transient ischemic attacks do not result in permanent brain injury through acute infarction (i.e., tissue death), but they can indicate serious risk of subsequent stroke.

The term “affinity” refers to the strength of a binding reaction between a binding domain of an antibody and an epitope. It is the sum of the attractive and repulsive forces operating between the binding domain and the epitope. The term affinity, as used herein, refers to the dissociation constant K_{D}.

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. In one embodiment, an epitope is a region of an antigen that is bound by an antibody. In certain embodiments, an antibody is said to specifically bind an antigen when it preferentially recognizes its target antigen in a complex mixture of proteins and/or macromolecules. Methods for epitope mapping are well known in the art, such as X-ray co-crystallography, array-based oligo-peptide scanning, site-directed mutagenesis, high throughput mutagenesis mapping and hydrogen-deuterium exchange. Epitopes can be formed both from contiguous amino acids, or noncontiguous amino acids juxtaposed by tertiary folding of a protein. Epitopes formed from contiguous ammo acid are typically retained on exposure to denaturing solvents, whereas epitopes formed by tertiary folding are typically lost on treatment with denaturing solvents. An epitope typically includes at least 3, and more usually, at least 5 or 8-10 amino acid in a unique spatial conformation.

The terms “reduce,” “inhibit” and “block” as used interchangeably herein, refer to any statistically significant decrease in biological activity (e.g., hemichannel opening). For example, “reduction” can refer to a decrease of about 10%, 20%, 30%, 40%, 50%, 60%, 70%, 80%, 90%, or 100% in biological activity.

### II. Antibodies

The term “antibody” or “antibodies” herein is used in the broadest sense and specifically covers full length antibody, antibody peptide(s) or immunoglobulin(s), monoclonal antibodies, chimeric antibodies, polyclonal antibodies, human antibodies, humanized antibodies and antibodies from non-human species, including human antibodies derived from a human germline immunoglobulin sequence transduced into the non-human species, e.g., mouse, sheep, chicken or goat, recombinant antigen binding forms such as monobodies and diabodies, multi-specific antibodies (e.g., bispecific antibodies), and individual antigen binding fragments of any of the foregoing, e.g., of an antibody or the antibody from which it is derived, including dAbs, Fv, scFv, Fab, F(ab)′2, Fab′.

Antibody-like binding peptidomimetics are also contemplated in the methods described herein. Liu et al. (2003) describe “antibody-like binding peptidomimetics” (ABiPs), which are peptides that act as pared-down antibodies and have certain advantages of longer serum half-life as well as less cumbersome synthesis methods.

The term “monoclonal antibody” as used herein refers to an antibody obtained from a population of substantially homogeneous antibodies, i.e., the individual antibodies comprising the population are identical and/or bind the same epitope. The modifier “monoclonal” indicates the character of the antibody as being obtained from a substantially homogeneous population of antibodies, and is not to be construed as requiring production of the antibody by any particular method.

“Antigen binding fragments” of an antibody preferably comprise at least the variable regions of the heavy and/or light chains of an anti-Cx43 antibody. For example, an antigen binding fragment of anti-Cx43 antibodies can comprise amino acid sequences of SEQ ID NOs: 7 and 8. Examples of such antigen binding fragments include Fab fragments, Fab′ fragments, Fv fragments, scFv and F(ab′)₂ fragments. Antigen binding fragments of an antibody can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a recombinant construct encoding the heavy chain of an F(ab′)₂ fragment can be designed to include DNA sequences encoding the CH1 domain and hinge region of the heavy chain. In one aspect, antigen binding fragments blocks or inhibits the opening of Cx43 hemi-channel in a subject and the effects associated with opening of Cx43 hemi-channel.

A “multi-specific antibody” as used herein refers to an artificial antibody that has two or more different portions, with each portion including the antigen binding region of an antibody to a different antigen or epitope. The portions of the multi-specific antibody can be, for example, full-length antibodies or antibody binding fragments. Bispecific or bifunctional antibodies are multi-specific antibodies that have two different portions (e.g., two different heavy and light chain pairs, or two different antibody binding fragments) that bind two different antigens or epitopes. Multi-specific antibodies can be produced by a variety of methods, including fusion of hybridomas or ligation of antibody binding fragments. For example, Songsivilai and Lachmann, Clin Exp Immunol, 79: 315-21 (1990); Kostelny et al., J. Immunol., See 148: 1547-53 (1992). In some cases, a multi-specific antibody (e.g., a bispecific antibody) includes at least one portion an anti-Cx43 antibody that blocks or inhibits the opening of Cx43 hemi-channel in a subject and the effects associated with opening of Cx43 hemi-channel.

A “therapeutic monoclonal antibody” is an antibody used for therapy of a human subject. Therapeutic monoclonal antibodies disclosed herein include anti-Cx43 antibodies. Antibody “effector functions” refer to those biological activities attributable to the Fc region (a native sequence Fc region or amino acid sequence variant Fc region) of an antibody. Examples of antibody effector functions include C1q binding, complement dependent cytotoxicity, Fc receptor binding, antibody-dependent cell-mediated cytotoxicity (ADCC), phagocytosis, down regulation of cell surface receptors (e.g., B cell receptor; BCR), and the like. To assess ADCC activity of a molecule of interest, an in vitro ADCC assay, such as those described in U.S. Pat. No. 5,500,362 or 5,821,337 may be performed.

An anti-Cx43 antibody of the disclosure can also be conjugated to at least one agent or moiety to form an antibody conjugate. The antibody may be linked to the agent covalently or non-covalently. The agent or moiety can increase the diagnostic or therapeutic potential of the antibody, and include, but are not limited to, effector or reporter molecules. Effector molecules include molecules with a desired activity (e.g., cytotoxic activity). Non-limiting examples of effector molecules that can be attached to an antibody include toxins, small molecule drugs, therapeutic enzymes, cytokines, antibiotics, radiolabeled nucleotides, and the like. Reporter molecules are molecules that can be detected by an assay, and include, without limitation, enzymes, radioactive labels, haptens, fluorescent labels, phosphorescent molecules, chemically luminescent molecules, chromophores, luminescent molecules, photoaffinity molecules, colored particles, or ligands (e.g., biotin).

Depending on the amino acid sequence of the constant domain of their heavy chains, full length antibodies can be assigned to different “classes.” There are five major classes of full-length antibodies: IgA, IgD, IgE, IgG, and IgM, and several of these may be further divided into “subclasses” (isotypes), e.g., IgGl, IgG2, IgG3, IgG4, IgA, and IgA2. The heavy-chain constant domains that correspond to the different classes of antibodies are called α, δ, ε, γ and μ, respectively. The subunit structures and three-dimensional configurations of different classes of antibodies are well known. The “light chains” of antibodies from any vertebrate species can be assigned to one of two clearly distinct types, called kappa (κ) and lambda (λ), based on the amino acid sequences of their constant domains.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association and is particularly not limited to chemical “conjugation.”

The term “hypervariable region” when used herein refers to the amino acid residues of an antibody which are responsible for antigen binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mal. Biol. 196:901-917 (1987)). “Framework Region” or “FR” residues are those variable domain residues other than the hypervariable region residues as herein defined. The hypervariable region or the CDRs thereof can be transferred from one antibody chain to another or to another protein to confer antigen binding specificity to the resulting (composite) antibody or binding protein.

As will be appreciated by those in the art, the CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

Amino acid substitutions are typically of single residues; insertions usually will be on the order of from about one to about twenty amino acid residues, although considerably larger insertions may be tolerated. Deletions range from about one to about twenty amino acid residues, although in some cases deletions may be much larger.

Substitutions, deletions, insertions or any combination thereof may be used to arrive at a final derivative or variant. Generally, these changes are done on a few amino acids to minimize the alteration of the molecule, particularly the immunogenicity and specificity of the antigen binding protein. However, larger changes may be tolerated in certain circumstances.

The term “Fab” or “Fab region,” as used herein, is meant the polypeptide that comprises the VH, CH1, VL, and CL immunoglobulin domains. Fab may refer to this region in isolation, or this region in the context of a full-length antibody, antibody fragment or Fab fusion protein, or any other antibody embodiments as outlined herein.

The term “Fv,” “Fv fragment” or “Fv region,” as used herein, refers to a polypeptide that comprises the VL and VH domains of a single antibody.

The term “framework,” as used herein, is refers to the region of an antibody variable domain exclusive of those regions defined as CDRs. Each antibody variable domain framework can be further subdivided into the contiguous regions separated by the CDRs (FR1, FR2, FR3 and FR4).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human antibody are replaced by corresponding non-human residues. Furthermore, humanized antibodies may comprise residues that are not found in the recipient antibody or in the donor antibody. These modifications are made to further refine antibody performance. For further details, see Jones et al., Nature, 321:522-525 (1986); Riechmann et al., Nature, 332:323-329 (1988); and Presta, Curr. Op. Struct. Biol., 2:593-596 (1992).

An “isolated” antibody is one which has been identified and separated and/or recovered from a component of its natural environment. In certain embodiments, the antibody will be purified (1) to greater than 95% by weight of protein as determined by the Lowry method, and alternatively, more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

### Anti-Cx43 Antibodies

Certain aspects of the present disclosure provide compositions and methods of treating a stroke in a subject. The methods and compositions comprise an anti-Cx43 antibody which specifically binds Cx43 hemichannel and blocks or inhibits the opening of it in cells.

The anti-Cx43 antibody can be any antibody specifically binds Cx43 known in the art. In various embodiments, the anti-Cx43 antibody used in the methods comprises an HCDR1 amino acid sequence having at least 90%, at least 95%, or at least 99% sequence identity to SEQ ID NO: 1, an HCDR2 amino acid sequence having at least 90%, at least 95%, or at least 99% sequence identity to SEQ ID NO: 2, and an HCDR3 amino acid sequence having at least 90%, at least 95%, or at least 99% sequence identity to SEQ ID NO: 3; and/or a LCDR1 amino acid sequence having at least 90%, at least 95%, or at least 99% sequence identity to SEQ ID NO: 4, a LCDR2 amino acid sequence having at least 90%, at least 95%, or at least 99% sequence identity to SEQ ID NO: 5, a LCDR3 amino acid sequence having at least 90%, at least 95%, or at least 99% sequence identity to SEQ ID NO: 6. In some specific embodiments, the anti-Cx43 antibody comprises an HCDR1 amino acid sequence identical to SEQ ID NO: 1, an HCDR2 amino acid sequence identical to SEQ ID NO: 2, and an HCDR3 amino acid sequence identical to SEQ ID NO: 3; and/or a LCDR1 amino acid sequence identical to SEQ ID NO: 4, a LCDR1 amino acid sequence identical to SEQ ID NO: 5, and a LCDR3 amino acid sequence identical to SEQ ID NO: 6.

In various embodiments, the anti-Cx43 antibody used in the method comprise a heavy chain having at least 80%, at least 85%, at least 90%, at least 95%, or at least 99% sequence identity to any one of SEQ ID NOs: 9 and 11-18; and/or a light chain having at least 80%, at least 85%, at least 90%, at least 95%, or at least 99% sequence identity to any one of SEQ ID NO: 10. In some specific embodiments, the anti-Cx43 antibody comprise a heavy chain having at least 80%, at least 85%, at least 90%, at least 95%, or at least 99% sequence identity to SEQ ID NO: 9; and a light chain having at least 80%, at least 85%, at least 90%, at least 95%, or at least 99% sequence identity to any one of SEQ ID NO: 10. In some other specific embodiments, the anti-Cx43 antibody comprise a heavy chain having at least 80%, at least 85%, at least 90%, at least 95%, or at least 99% sequence identity to SEQ ID NO: 11; and a light chain having at least 80%, at least 85%, at least 90%, at least 95%, or at least 99% sequence identity to any one of SEQ ID NO: 10. In some other specific embodiments, the anti-Cx43 antibody comprise a heavy chain having at least 80%, at least 85%, at least 90%, at least 95%, or at least 99% sequence identity to SEQ ID NO: 12; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 13; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 14; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 15; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 16; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 17; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 18; and a light chain having at least 80%, at least 85%, at least 90%, at least 95%, or at least 99% sequence identity to any one of SEQ ID NO: 10.

In some embodiments, the anti-Cx43 antibody comprises a heavy chain identical to any one of SEQ ID NOs: 9 and 11-18, and/or a light chain identical to any one of SEQ ID NO: 10. In a preferred embodiment, the anti-Cx43 antibody comprises a heavy chain identical to SEQ ID NO: 9, and a light chain identical to any one of SEQ ID NO: 10. In one embodiment, the anti-Cx43 antibody comprises a heavy chain identical to SEQ ID NO: 11, and a light chain identical to any one of SEQ ID NO: 10. In another embodiment, the anti-Cx43 antibody comprises a heavy chain identical to SEQ ID NO: 12, and a light chain identical to any one of SEQ ID NO: 10. In yet another embodiment, the anti-Cx43 antibody comprises a heavy chain identical to SEQ ID NO: 13, and a light chain identical to any one of SEQ ID NO: 10. In yet another embodiment, the anti-Cx43 antibody comprises a heavy chain identical to SEQ ID NO: 14, and a light chain identical to any one of SEQ ID NO: 10. In yet another embodiment, the anti-Cx43 antibody comprises a heavy chain identical to SEQ ID NO: 15, and a light chain identical to any one of SEQ ID NO: 10. In yet another embodiment, the anti-Cx43 antibody comprises a heavy chain identical to SEQ ID NO: 16, and a light chain identical to any one of SEQ ID NO: 10. In yet another embodiment, the anti-Cx43 antibody comprises a heavy chain identical to SEQ ID NO: 17, and a light chain identical to any one of SEQ ID NO: 10. In yet another embodiment, the anti-Cx43 antibody comprises a heavy chain identical to SEQ ID NO: 18, and a light chain identical to any one of SEQ ID NO: 10.

In various embodiments, provided herein is an antibody that binds an epitope located within, partially or entirely, the amino acid sequence of FLSRPTEKTI (SEQ ID NO: 19). In some embodiments, the epitope can comprise one or more amino acids selected from the group consisting of F1, S3, R4, P5, T6, E7, K8, T9, or I10 of SEQ ID NO: 19. In one embodiment the epitope consists of F1, S3, R4, P5, T6, E7, K8, T9 and 110 of SEQ 1D NO: 19. In some embodiments, the epitope can include all ten amino acids of SEQ ID NO: 19. In certain embodiments, the epitope consists of all ten amino acids of SEQ ID NO: 19.

The anti-Cx43 antibody is substantially pure and desirably substantially homogeneous (i.e., free from contaminating proteins, etc.). “Substantially pure” antibody means a composition comprising at least about 90% antibody by weight, based on total weight of the protein in the composition, at least about 95% or 97% by weight. “Substantially homogeneous” antibody means a composition comprising protein wherein at least about 99% by weight of protein is specific antibody, e.g., anti-Cx43 antibody, based on total weight of the protein.

In some embodiments, the anti-Cx43 antibody is a humanized antibody. In some embodiments, the anti-Cx43 antibody is a monoclonal antibody. In some embodiments, the anti-Cx43 antibody is a humanized monoclonal antibody.

### Pharmaceutical Formulation Comprising Anti-Cx43 Antibody

One aspect of the present disclosure provides a pharmaceutical formulation for treating a stroke in a subject comprising an anti-Cx43 antibody. In some embodiments, the anti-Cx43 antibody blocks or inhibits the opening of Cx43 hemichannels in cells. The anti-Cx43 antibody can be a full antibody or an antigen-binding fragment thereof.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active agent (e.g., an anti-Cx43 antibody) may be coated m a material to protect the active agent from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, or by the use of surfactants, such as polysorbate, sodium dodecyl sulfate, and nonionic surfactant. In many cases, it may be useful to include isotonic agents, for example, sugars, polyalcohols such as mannitol, sorbitol, or sodium chloride in the composition. Prolonged absorption of the injectable compositions can he brought about by including in the composition, an agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration. Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol phenol sorbic acid, and the like. it may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions described herein can also have various viscosities or osmolarities. Methods of measuring viscosity of antibody formulations are known to those in the art, and can include, e.g., a rheometer (e.g., Anton Paar MCR301 Rheometer with either a 50 mm, 40 mm or 20 mm plate accessory). Methods of measuring osmolarity of antibody formulations are known to those in the art, and can include, e.g., an osmometer (e.g., an Advanced Instrument Inc 2020 freezing point depression osmometer).

The pharmaceutical compositions described herein can also have various pH levels. The pH of the pharmaceutical composition can be adjusted by any method known in the art, such as, for example, addition of a buffer.

In some embodiments, the pharmaceutical composition includes an anti-Cx43 antibody described herein, a histidine/histidine hydrochloride buffer, Polysorbate 80, and sucrose. In some embodiments, the pharmaceutical composition includes about 40 mg/mL to about 60 mg/mL of an anti-Cx43 antibody described herein; about 10 mM to about 40 mM histidine/histidine hydrochloride buffer; about 0.005% w/v to about 0.05% w/v Polysorbate 80; and about 1% w/v to about 20% w/v sucrose. In some embodiments, the pharmaceutical composition includes about 50 mg/mL of an anti-Cx43 antibody described herein; about 20 mM histidine/aspartic acid buffer; about 0.02% w/v Polysorbate 80; and about 8% w/v sucrose. In some embodiments, the pharmaceutical composition has a pH of between about 5.4 to about 5.6. In some embodiments, the pharmaceutical composition has a pH of about 5.5.

It may be advantageous to formulate parenteral compositions or unit dosage form for ease of administration and uniformity of dosage. Unit dosage form as used herein refers to physically discrete units suited as unitary dosages for the patients to be treated: each unit contains a predetermined quantity of active agent calculated to produce the desired therapeutic effect in association with any required pharmaceutical carrier. The specification for unit dosage forms is dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitations, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Intravenous injection and infusion are often (but not exclusively) used for antibody administration.

The formulations described herein are administered to a subject in need of treatment with the anti-Cx43 antibodies, preferably a human, in accord with known methods, such as intravenous administration as a bolus or by continuous infusion over a period of time, by intramuscular, intradermal, intraperitoneal, intracerobrospinal, subcutaneous, intra-articular, intrasynovial, intrathecal, oral, topical, or inhalation routes.

In some embodiments, the anti-Cx43 antibodies are administered to a subject by intravenous or subcutaneous (i.e., beneath the skin) administration. For such purposes, the formulation may be injected using a syringe. However, other devices for administration of the formulation are available such as injection devices (e.g., the INJECT-EASE™ and GENJECT™ devices); injector pens (such as the GENPEN™); auto-injector devices, needleless devices (e.g., MEDIJECTOR™ and BIOJECTOR™); and subcutaneous patch delivery systems.

In a specific embodiment, the present disclosure is directed to kits for a single dose-administration unit. Such kits comprise a container of an aqueous formulation of therapeutic protein or antibody, including both single and multi-chambered pre-filled syringes. Exemplary pre-filled syringes are available from Vetter GmbH, Ravensburg, Germany.

### III. Method of Treating a Stroke

Certain aspects of the present disclosure provide methods of treating a stroke in a subject. The methods can comprise blocking the opening of Cx43 hemichannels in cells in a subject in need thereof by using, e.g., an anti-Cx43 antibody. The Cx43 hemichannel modulation can be a stand-alone therapy for the stroke, or in conjunction with other therapies.

A stroke involves the acute loss of brain function due to loss of normal Hood supply to the brain, brainstem, spinal cord, or retina. Occurrence of a stroke in a subject often results in death of brain tissue and swelling, which can in turn lead to impairments in neurological function. In severe cases, a stroke can lead to death of the subject, either directly, or indirectly resulting from complications associated with the stroke. In some embodiments, the stroke is an ischemic stroke. In some embodiments, the ischemic stroke is an embolic stroke, a cardioembolic stroke, a thrombotic stroke, a large vessel thrombosis, a lacunar infarction, an artery-artery stroke, or a cryptogenic stroke. In other embodiments, the stroke is a hemorrhagic stroke. In some embodiments, the hemorrhagic stroke is a subdural stroke, an intraparenchymal stroke, an epidural stroke, or a subarachnoid stroke.

Various cells are able to communicate with each other and with the extracellular environment through hemichannels and gap junctions formed by the protein connexin. Connexin proteins are ubiquitously expressed throughout the body. Six connexin proteins make up one hemichannel, and two hemichannels make up one gap junction channel. Gap junctions are a cluster of channels that are located in the plasma membrane between adjoining cells, and they mediate intercellular communication. Hemichannels are a separate entity from gap junction channels. Hemichannels permit the exchange of molecules between the intracellular compartments and the extracellular environment.

Some cells in the brain express hemichannels known as connexin (Cx) 43 hemichannels. Under normal conditions, Cx43 hemichannels in astrocytes remain closed. Inflammatory conditions and mechano-stimulation can induce opening of the Cx43 hemichannels, which leads to the release of pro-inflammatory factors, and contributes to the loss of ion and neurotransmitter homeostasis and swelling associated with a stroke. Inhibiting the opening of Cx43 hemichannels in cells (e.g., using an anti-Cx43 antibody) can thus ameliorate the effects of a stroke in a subject.

Cx43 is also known as gap junction alpha-1 protein (GJA1), which is a 43.0 kDa protein composed of 382 amino acids (NCBI Reference Sequence: NP 000156.1). GJA1 contains a long C-terminal tail, an N-terminal domain, and multiple transmembrane domains. The protein passes through the phospholipid bilayer four times, leaving its C- and N-terminals exposed to the cytoplasm. The C-terminal tail is composed of 50 amino acids and includes post-translational modification sites, as well as binding sites for transcription factors, cytoskeleton elements, and other proteins. As a result, the C-terminal tail is central to functions such as regulating pH gating and channel assembly. Notably, the DNA region of the GJA1 gene (NCBI Gene ID: 2697) encoding this tail is highly conserved, indicating that it is either resistant to mutations or becomes lethal when mutated. Meanwhile, the N terminal domain is involved in channel gating and oligomerization and, thus, may control the switch between the channel's open and closed states. The transmembrane domains form the gap junction channel while the extracellular loops facilitate proper channel docking. Moreover, two extracellular loops form disulfide bonds that interact with two hexamers to form a complete gap junction channel.

“Treatment” refers to therapeutic treatment. Those in need of treatment include those already with disease. Hence, the subject, e.g., a human, to be treated herein may have been diagnosed as suffering from a disease, such as osteoarthritis. A disease, e.g., osteoarthritis, is “inhibited” or “treated” if at least one symptom (as determined by responsiveness/non-responsiveness, or indicators known in the art and described herein) of the condition is alleviated, terminated, slowed, minimized, or prevented. The terms “patient” and “subject” are used interchangeably herein.

The term “subject” or “patient” refers to either a human or non-human, such as primates, mammals, and vertebrates. In particular embodiments, the subject is a human.

Treatment can be suitably administered to subjects, particularly humans, suffering from, having, susceptible to, or at risk of developing osteoarthritis. Determination of those subjects “at risk” can be made by any objective or subjective determination by a diagnostic test or opinion of a subject or health care provider. Identifying a subject in need of such treatment can be in the judgment of a subject or a health care professional and can be subjective (e.g., opinion) or objective (e.g., measurable by a test or diagnostic method).

The anti-Cx43 antibody for use in any of the methods disclosed herein can be stored as a lyophilized solid or an aqueous formulation, or any other forms known in the art. In the case of an anti-Cx43 antibody which is stored as a lyophilized solid, the antibody is reconstituted in a solution such as water (e.g., for injection) prior to administration. If prepared for infusion either from a lyophilized form or an aqueous formulation, the final concentration, e.g., after dilution of the reconstituted antibody (e.g., in a saline, Ringer's or 5% dextrose infusion system) of the anti-Cx43 antibody can be about 0.1 mg/ml to about 80 mg/ml for administration. The final concentration may be about 0.1 mg/ml to about 80 mg/ml, about 0.5 mg/ml to about 70 mg/ml, about 1 mg/ml to about 60 mg/ml, about 5 mg/ml to about 50 mg/ml, about 10 mg/ml to about 40 mg/ml, about 15 mg/ml to about 30 mg/ml, or about 20 mg/ml to about 25 mg/ml. In some embodiments, the final dosage form may be at a concentration of about 0.1 mg/ml, about 0.5 mg/ml, about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml or higher than 80 mg/ml.

The term “effective amount,” as used herein, refers to that amount of an agent, such as an anti-Cx43 antibody, which is sufficient to effect treatment, prognosis or diagnosis of osteoarthritis, when administered to a patient or a subject. Dosage regimens may be adjusted to provide the optimum therapeutic response. An effective amount is also one in which any toxic or detrimental effects (side effects) of the agent are minimized and/or outweighed by the beneficial effects. A therapeutically effective amount will vary depending upon the patient and disease condition being treated, the weight and age of the patient, the severity of the disease condition, the manner of administration, course of the condition, patient's clinical history and response to anti-Cx43 antibody and the like, which can readily be determined by one of ordinary skill in the art. For example, an effective amount or a dose of the anti-Cx43 antibody ranges from about 0.01 mg/kg to about 1000 mg/kg. In some embodiments, the effective amount or the dose of the anti-Cx43 antibody is about 0.01 mg/kg to about 900 mg/kg, about 0.1 mg/kg to about 800 mg/kg, about 0.5 mg/kg to about 700 mg/kg, about 1 mg/kg to about 600 mg/kg, about 1.5 mg/kg to about 500 mg/kg, about 2 mg/kg to about 400 mg/kg, about 5 mg/kg to about 300 mg/kg, about 10 mg/kg to about 200 mg/kg, about 15 mg/kg to about 100 mg/kg, about 20 mg/kg to about 50 mg/kg, about 25 mg/kg to about 45 mg/kg, or about 30 mg/kg to about 40 mg/kg. In some embodiments, the effective amount or the dose of the anti-Cx43 antibody is about about 0.01 mg/kg, about 0.1 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, or more than 1000 mg/kg.

In specific embodiments, an effective amount or a dose of the anti-Cx43 antibody is about 0.01 mg/kg to about 100 mg/kg. In some embodiments, the effective amount or a dose of the anti-Cx43 antibody is 15 mg/kg. In some embodiments, the effective amount or a dose of the anti-Cx43 antibody is 25 mg/kg. In some embodiments, the effective amount or a dose of the anti-Cx43 antibody is 50 mg/kg.

In some embodiments, an effective amount or a dose of the anti-Cx43 antibody can range from about 1 mg to about 800 mg. In some embodiments, the effective amount or the dose of the anti-Cx43 antibody is about 2 mg to 7000 mg, about 5 mg to 6000 mg, about 10 mg to 5000 mg, about 15 mg to 4000 mg, about 20 mg to 3000 mg, about 25 mg to 2000 mg, about 30 mg to 1000 mg, about 40 mg to 900 mg, about 50 mg to 800 mg, about 60 mg to 700 mg, about 70 mg to 600 mg, about 80 mg to 500 mg, about 90 mg to 400 mg, about 100 mg to 300 mg, or about 150 mg to 250 mg. In some embodiments, the effective amount or the dose of the anti-Cx43 antibody is less than 1 mg, about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1250 mg, about 1500 mg, about 1750 mg, about 2000 mg, about 2250 mg, about 2500 mg, about 2750 mg, about 3000 mg, about 4000 mg, about 5000 mg, about 6000 mg, about 7000 mg, about 8000 mg, or more than 8000 mg.

In the methods as described herein, an effective amount or at least one dose of the anti-Cx43 antibody is administered about once every day, about once every 2 days, about once every 3 days, about once every 4 days, about once every 5 days, about once every 6 days, about once every week, about once every 8 days, about once every 9 days, about once every 10 days, about once every 11 days, about once every 12 days, about once every 13 days, about once every 2 weeks, about once every 15 days, about once every 16 days, about once every 17 days, about once every 18 days, about once every 19 days, about once every 20 days, about once every 3 weeks, about once every 22 days, about once every 23 days, about once every 24 days, about once every 25 days, about once every 26 days, about once every 27 days, about once every 4 weeks, about once every 29 days, about once every 30 days, about once every 31 days, about once every 32 days, about once every 33 days, about once every 34 days, about once every 5 weeks, about once every 36 days, about once every 37 days, about once every 38 days, about once every 39 days, about once every 40 days, or about once every 41 days, about once every 6 weeks, about once every 7 weeks, about once every 8 weeks, about once every 9 weeks, about once every 10 weeks, about once every 11 weeks, about once every 12 weeks, about once every 13 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks or 6 months, or about once every more than 24 weeks or 6 months after the stroke.

In the methods as described herein, an effective amount or at least one dose of the anti-Cx43 antibody is administered within 15 minutes, within 30 minutes, within 1 hour, within 2 hours, within 3 hours, within 4 hours, within 5 hours, within 6 hours, within 7 hours, within 8 hours, within 9 hours, within 10 hours, within 11 hours, within 12 hours, within 24 hours, within 36 hours, within 48 hours, within 60 hours, within 72 hours, within 4 days, within 5 days, within 6 days, within 1 week, within 2 weeks, within 3 weeks, within 4 weeks, within 5 weeks, within 6 weeks, within 7 weeks, within 8 weeks, within 9 weeks, within 10 weeks, within 11 weeks, within 12 weeks or within more than 12 weeks from the stroke.

In some specific embodiments, an effective amount or a dose of the anti-Cx43 antibody is administered within one week from the stroke. In one particular embodiment, a dose of the anti-Cx43 antibody is administered within 30 minutes, within 2 hours, or within 6 hours from the stroke.

According to certain embodiments of the present disclosure, multiple doses of an anti-Cx43 antibody (or a pharmaceutical composition comprising a combination of an anti-Cx43 antibody and any of the additional therapeutically active agents mentioned herein) may be administered to a subject over a defined time course. The methods according to this aspect of the disclosure comprises sequentially administering multiple doses of an anti-Cx43 antibody of the disclosure to a subject.

As used herein, “sequentially administering” means that each dose of anti-Cx43 antibody is administered to the subject at a different point in time, e.g., on different days separated by a predetermined interval (e.g., hours, days, weeks or months). The present disclosure includes methods which comprise sequentially administering to the patient a single initial dose of an anti-Cx43 antibody, followed by a second dose of the anti-Cx43 antibody, and optionally followed by one or more subsequent doses of the anti-Cx43 antibody. The anti-Cx43 antibody may be administered at a dose of between 0.01 mg/kg to about 1000 mg/kg.

The terms “first dose,” and “second dose” refer to the temporal sequence of administration of the anti-Cx43 antibody of the disclosure. Thus, the “first dose” is the dose which is administered at the beginning of the treatment regimen (also referred to as the “baseline dose”); and the “second doses” are the doses which are administered after the first dose. The first and second may all contain the same amount of anti-Cx43 antibody, but generally may differ from one another in terms of frequency of administration. In certain embodiments, however, the amount of anti-Cx43 antibody contained in the first, second, and/or doses following the second dose varies from one another (e.g., adjusted up or down as appropriate) during the course of treatment.

In some embodiments of the methods of the disclosure, the method comprises administering a first dose and a second dose of the anti-Cx43 antibody. In some embodiments, the first dose of the anti-Cx43 antibody is administered within 1 week from the stroke. In some embodiments, the first dose of the anti-Cx43 antibody is administered within 30 minutes, 2 hours, or 6 hours from the stroke. In some embodiments, the second dose of the anti-Cx43 antibody is administered a day to 4 weeks after the first dose is administered. In some embodiments, the second dose of the anti-Cx43 antibody is administered at least 4 days after the first dose is administered. In some embodiments, the method further comprises administering one or more subsequent doses of the anti-Cx43 antibody after the second dose is administered.

In some embodiments of the methods of the disclosure, the method comprises administering an effective amount of an anti-Cx43 antibody according to the following dose regimens: a first dose within one week from the stroke, and a second dose within a month from the stroke. In some embodiments, the method further comprises administering subsequently doses of the anti-Cx43 antibody weekly after the fourth dose.

The effective amount or at least one dose of the anti-Cx43 antibody is suitably administered to the patient at one time or over a series of treatments and may be administered to the patient at any time from diagnosis onwards. The anti-Cx43 antibody may be administered as the sole treatment or in conjunction with other drugs or therapies useful in treating a stroke.

In the methods as described herein, an effective amount or a dose of the anti-Cx43 antibody can be administered to a patient over less than 5 minutes, about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more than 180 minutes. In some specific embodiments, the effective amount or the dose of the anti-Cx43 antibody can be administered to a patient over about 30 minutes. In one specific embodiment, the effective amount or the dose of the anti-Cx43 antibody can be intravenously administered to a patient over 30 minutes.

In the methods described herein, the methods include preparing the anti-Cx43 antibody for administration to a patient. If the anti-Cx43 antibody is in a formulation which is in a solid, e.g., dry state, the process of administration can comprise a step of converting the formulation to a liquid state. In one aspect, a dry formulation can be reconstituted, e.g., by a liquid as described above, for use in injection, e.g., intravenous, intradermal, intramuscular, intraperitoneal or subcutaneous injection. In another aspect, a solid or dry formulation can be administered topically, e.g., in a patch, cream, aerosol or suppository.

In the methods described herein, the anti-Cx43 antibody may be administered to a subject alone or in conjunction with another therapy. The anti-Cx43 antibody can be administered before, along with or subsequent to administration of the additional therapy. In one embodiment, the dose of the co-administered therapy can be decreased over time or completely tapered during the period of treatment by the anti-Cx43 antibody. In some embodiments, the additional comprises one or more of an anti-coagulant, a steroid, a surgery (e.g., mechanical clot retrieval), a cell-based therapy, a small molecule drug, a peptide, a hormone, a statin, a growth factor, a neuroprotective therapy, an antibody, electrical stimulation, an intravenous fibrinolytic (clot dissolving) therapy, or an intra-arterial fibrinolysis therapy.

In one aspect, the improved effectiveness of a combination according to the disclosure can be demonstrated by achieving therapeutic synergy. The term “therapeutic synergy” or “synergistic effect” is used when the combination of two products at given doses is more efficacious than the best of each of the two products alone at the same doses. In one example, therapeutic synergy can be evaluated by comparing a combination to the best single agent using estimates obtained from a two-way analysis of variance with repeated measurements (e.g., time factor) on an osteoarthritis severity parameter

The patient or subject, before, during, or after being treated with the anti-Cx43 antibody, is examined for stroke-related parameters using technologies and methods known in the art. Non-limiting examples of the common medical technologies and methods used to examine and diagnose a stroke include: physical examination by a physician, physical performance tests, blood tests, magnetic resonance imaging (MRI) radiography, including diffusion and perfusion MRI imaging, flow-sensitive imaging, such as fluid-attenuated inversion recovery (FLAIR), functional and spectroscopical imaging, positron emission tomography (PET), the Cincinnati Stroke Scale and the Los Angeles Prehospital Stroke Screen (LAPSS), the Canadian Neurological Scale (CNS), the Glasgow Coma Scale (GCS), the Hempispheric Stroke Scale, the Hunt and Hess Scale, the Mathew Stroke Scale, the Mini-Mental State Examination (MMSE), the National Institute of Health Stroke Scale (NIHSS), the Orgogozo Stroke Scale, the Oxfordshire Community Stroke Project Classification (Bamford), the Scandinavian Stroke Scale, the Berg Balance Scale, the Modified Rankin Scale, the Stroke Impact Scale (SIS), and the Stroke Specific Quality of Life Measure (SS-QOL), the American Heart Association Stroke Outcome Classification (AHA SOC), the Barthel Index, the Functional Independence Measurement (FIM), the Glasgow Outcome Scale (GOS), the Health Survey Short Form 36 (SF36) and Short Form 12 (SF12), the Action Research Arm Test, the Blessed-Dementia Scale, the Blessed-Dementia Information-Memory-Concentration Test, the DSM-IV criteria for the diagnosis of vascular dementia, the Hachinkski Ischemia Score, the Hamilton Rating Scale for Depression, the NINDS-AIREN criteria for the diagnosis of vascular dementia, the Orpington Prognostic Score, the Short Orientation-Memory-Concentration Test, and the Thrombosis In Myocardial Infarction grading scheme. The choice of technologies and methods, and the frequency of examination can be determined and/or adjusted by a person skilled in the art based on the patient's or subject's specific conditions.

In some embodiments, the subject, before, during, or after being treated with the anti-Cx43 antibody, is examined for improvement of the stroke as assessed by at least one post-stroke criterion. In some embodiments, at least post-stroke criterion is assessed within three to six months after the anti-Cx43 antibody is administered. In some embodiments, at least post-stroke criterion is assessed at least one day to two weeks after the anti-Cx43 antibody is administered. In some embodiments, at least post-stroke criterion is assessed at least one to two days after the anti-Cx43 antibody is administered. In some embodiments, at least one indicator of osteoarthritis severity is assessed at least one day, at least two days, at least three days, at least four days, at least five days, at least six days, or at least a week after anti-Cx43 antibody is administered to the subject. In some embodiments, at least one post-stroke criterion comprises neurological function, cerebral infarct volume, and/or cerebral edema. In some embodiments, at least one post-stroke criterion is improved after the anti-Cx43 antibody is administered.

In some embodiments, treatment with the anti-Cx43 antibody for stroke improves the disease symptoms as assessed by neurological function, cerebral infarct volume, and/or cerebral edema compared with a control, which can include but is not limited to a stroke population who receive or failed the same lines of therapy as the subject, but do not receive the anti-Cx43 antibody for stroke.

In some embodiments, the subject treated with the anti-Cx43 antibody for stroke has a duration of response of at least 1 day, at least 2 days, at least 3 days, at least 4 days, at least 5 days, at least 10 days, at least 15 days, at least 20 days, at least 25 days, at least 1 month, at least 2 months, at least 3 months, or more.

In some embodiments, the subject treated with the anti-Cx43 antibody for stroke has a time to response less than 3 months, less than 2 months, less than 1 month, less than 25 days, less than 20 days, less than 15 days, less than 10 days, less than 7 days, less than 6 days, less than 5 days, less than 4 days, less than 3 days, less than 2 days, or less than 1 day.

As used herein, “neurological function” refers to how the brain receives and sends information to the rest of the body. Neurological function can include movement and sensation, speech, thinking, reasoning, memory, vision, and emotions. Multiple factors can affect neurological function of a subject, including injury or disease (e.g., a stroke). In the case of injury or disease (e.g., a stroke), the disruption of neurological function is dependent on the size and location of the affected area in the brain. Neurological function, and disruptions thereof, can be assessed by any suitable method known in the art.

In some embodiments, treatment of a stroke in a subject with at least one dose of the anti-Cx43 antibody improves the neurological function of the subject. In some embodiments, treatment of a stroke in a subject with at least one dose of the anti-Cx43 antibody improves the neurological function of the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. In some embodiments, the neurological function is improved by at least one metric of neurological function.

As used herein, the term “infarction” refers to tissue death resulting from restricted blood supply to a region. An infarction can result from injury, blockage of blood supply to the tissue, or disease (e.g., a stroke). The “cerebral infarct volume” refers to infarcted volume of the brain, e.g., following a stroke. The cerebral infarct volume can be measured by any suitable method known in the art.

In some embodiments, treatment of a stroke in a subject with at least one dose of the anti-Cx43 antibody reduces the cerebral infarct volume in the subject. In some embodiments, treatment of a stroke in a subject with at least one dose of the anti-Cx43 antibody reduces the cerebral infarct volume cerebral edema in the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more.

As used herein, the “cerebral edema” refers to the swelling of the brain. Cerebral edema can result from injury to the brain or disease (e.g., a stroke), and can lead to debilitating complications in a subject. Cerebral edema can be categorized as vasogenic (resulting from disruption of the blood-brain barrier), cellular or cytotoxic ((resulting from disruption of glia, neurons or endothelial cells in the brain), interstitial (resulting from the outflow of cerebrospinal fluid from the intraventricular space to the interstitial areas of the brain), or osmotic (resulting from disrupted osmolarity and increased water content in the brain). Cerebral edema can be diffused, affecting multiple areas of the brain, or focal, affecting just one region of the brain. Any suitable method known in the art may be used to assess cerebral edema.

In some embodiments, treatment of a stroke in a subject with at least one dose of the anti-Cx43 antibody reduces cerebral edema in the subject. In some embodiments, treatment of a stroke in a subject with at least one dose of the anti-Cx43 antibody reduces cerebral edema in the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. In some embodiments, the cerebral edema is reduced by at least one metric of cerebral edema. In some embodiments, the at least one metric of cerebral edema comprises brain volume or brain water content.

As used herein, a “response” or “being responsive” to a treatment refers to the subject having improvement of at least one parameter of disease progression. The subject can have partial response or complete response to a treatment. The response to a treatment can be determined based on methods known in the art. A person skilled in the art can determine the proper methods based on the type of diseases being evaluated. Non-limiting examples of the methods include neurological function, cerebral infarct volume, and cerebral edema, as well as other methods described herein.

### EXAMPLES

The following examples are included to demonstrate preferred embodiments of the invention. It should be appreciated by those of skill in the art that the techniques disclosed in the examples which follow—represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Effects of Treatment with an Anti-Cx43 Antibody on a Mouse Model of Stroke

This Example describes the evaluation of an anti-Cx43 antibody for the treatment of stroke in a mouse model of stroke.

### Study Objective

The purpose of the study was to test the effect of the anti-Cx43 antibody ALMB-0166 on stroke symptoms in a mouse model of stroke induced by middle cerebral artery occlusion (MCAO).

### Regulatory Compliance

The experiment complied with Standard Operating Procedures (SOPs).

### Materials and Methods

### Test Materials

The anti-Cx43 antibody used in the study was ALMB-0166 (AlaMab Therapeutics Inc.). Butylphthalide and sodium chloride (CSPC NBP Pharmaceutical Group Co., Ltd.) was used as a positive control. Other materials used in the study included 0.9% normal saline (Anhui Shuanghe Pharmaceutical Co., Ltd). sterile water for injection (Sichuan Kelun Pharmaceutical Co., Ltd.), isoflurane (RWD Life Science Inc.), phosphate buffered saline (Thermo, batch no.), 2,3,5-triphenyltetrazolium chloride (TTC; Bide Pharmaceutical Technology, Co., Ltd.), and a 40% formaldehyde solution (Sinopharm Chemical Reagent Co., Ltd.)

For animal dosing, solutions of ALMB-0166 at 1 mg/mL, 2.5 mg/mL and 5 mg/mL were prepared by diluting the antibody stock solution with PBS. A 1% w/v TTC solution was also prepared for the study.

### Equipment

Equipment used in this study included a inhalation anesthesia apparatus (Midmark Corporation, type VWR), a mouse thread bolt (Beijing Cinontech Co., Ltd., type A4-162450), and a mouse brain mold (RWD Life Sciences Inc., type 175-800).

### Animals and Husbandry

Male Institute of Cancer Research (ICR) mouse (Specific Pathogen Free grade; Shanghai Bikai Keyi Biotechnology Co. Ltd) were obtained in the study. Each mouse was weight at the start of treatment, with the average weight ranging from 25-30 grams. Upon arrival at the test facility, the animals were housed at five mice per cage and acclimated for three to five days.

Animals were kept in an environment set to maintain a temperature of 23±2° C., with humidity of 40-70%, and a 12-hour light/12-hour dark cycle. The 12-hour dark cycle was temporarily interrupted to accommodate study procedures. SPF Rat Growth Breeding Feed (Beijing Keao Xieli Feed Co., Ltd)) was provided ad libitum throughout the in-life part of the study. Reverse Osmosis water was available ad libitum.

The animals used in the study were selected based on overall health and acclimation to caging. The mice were fasted for 12 hours before experimental modeling and were free to access both food and water afterwards.

A total of 72 rats were selected for participation in the study. Studies using common mammalian laboratory animals such as mice, dogs, and monkeys are essential and routinely used for the evaluation of the pharmacokinetics, pharmacodynamics and toxicology characteristics of new chemical entities. The number of animals in each test group was the minimal number needed for the assessment of variability between test animals.

### Study Design

### Animal Groups and Drugs

For the study, animal groups were formed as indicated in Table 1 below. The administration of the respective treatments was started 30 minutes after the MCAO procedure of the mice.

TABLE 1
Treatment and dosing information for animal test groups.

| | | | | | | Dosing route, |
|---|---|---|---|---|---|---|
| | | Animal | Dose | Dose volume | Concentration | frequency, and |
| Group | Treatment | Number | (mg/kg) | (mL/kg) | (mg/mL) | period |
| 1 | Sham (no stroke) | 12 | — | — | — | — |
| 2 | Model (vehicle- | 12 | — | 10 | — | Intravenous |
| | treated) | | | | | (i.v.), once |
| 3 | Butylphthalide | 12 | 10 | 20 | 0.25 | Intravenous |
| | 10 mg/kg | | | | | (i.v.), twice |
| | | | | | | daily (Bid) |
| 4 | ALMB-0166 15 | 12 | 15 | 10 | 1.5 | Intravenous |
| | mg/kg | | | | | (i.v.), once |
| 5 | ALMB-0166 25 | 12 | 25 | 10 | 2.5 | Intravenous |
| | mg/kg | | | | | (i.v.), once |
| 6 | ALMB-0166 50 | 12 | 50 | 10 | 5 | Intravenous |
| | mg/kg | | | | | (i.v.), once |

### Models and Methods

Seventy-two (72) ICR mice received behavioral training after adaptive feeding period and before the operation to establish stroke models. The behavioral training included beam-walking and rotarod test. The experimental mice were then randomly divided into 6 groups according to the values of the body weight, with 12 mice in each test group as described in Table 1.

The mice were fasted overnight before modeling and were anesthetized with isoflurane. Right middle cerebral artery occlusion (MCAO) was induced in mice using the intraluminal filament technique to mimic focal cerebral ischemic stroke. After 1 hr, reperfusion was allowed by removing the occlusion. During the operation, a white light was used to maintain the mouse rectal temperature between 36.5° C.-37.0° C.

Administration of each treatment was performed 0.5 h after the infarction. The neurological function score was evaluated at 24 hrs and 48 hrs after reperfusion, as was assessed by beam-walking, grip strength, and rotarod tests. At 48 h after reperfusion, the whole brain tissue of all rats was harvested to assess the cerebral infarct area by TTC staining, as well as to measure brain volume and brain water content, two indicators of acute cerebral edema.

### Statistical Analysis

The neurological function scores, the cerebral infarct volume, the brain volume, and the brain water content were expressed as the mean and standard error (Mean±SEM). The data between model group and administration (treatment) group at multiple time points were analyzed by repeated measures analysis of variance method with SPSS Statistics 21 software, and Tamhane's method for subsequent analysis. p<0.05 was considered to have a significant difference, and p<0.01 was considered to have a very significant difference. The final data were plotted with GraphPad Prism 6 software.

### Results

General Clinical Symptoms of Mice after Administration

All animals had no visible abnormalities during the experimental period.

### Effect of Treatment with Anti-Cx43 Antibody on Beam-Walking of Mice after MCAO

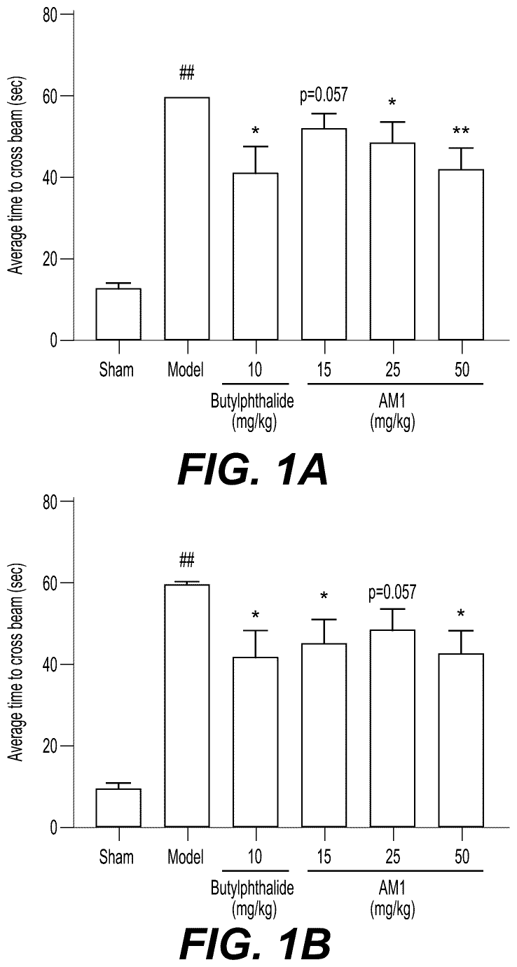

Beam-walking test was used to assess neurological function the mouse stroke model after the stroke. The beam-walking time of each test group after 24- and 48-hours post-operation are shown in Table 2 and FIGS. 1A-1B. A statistical difference was observed in the beam-walking time of the model (vehicle-treated) test group as compared to the sham group at both 24- and 48-hours post-operation (p<0.01; Table 2). At 24 hours post-operation, the butylphthalide and the ALMB-0166 25 mg/kg and 50 mg/kg groups showed a significant decrease in beam walking time, as compared to the model test group (p<0.05, p<0.05, and p<0.01, respectively; Table 2 and FIG. 1A). A difference was also observed between the ALMB-0166 15 mg/kg test group and the model test group at 24 hours (p=0.57). At 48 hours post-operation, a significantly lower beam walking time was observed for the butylphthalide and the ALMB-0166 mg/kg and 50 mg/kg test groups, as compared to the model test group (p<0.05; Table 2 and FIG. 1B). A difference was also observed between the ALMB-0166 25 mg/kg test group and the model test group at 24 hours (p=0.57).

TABLE 2
Beam-walking time of animal test groups over
study period (Mean ± SEM, n = 12)

| | Time (s) | |
|---|---|---|
| Group | 24 h | 48 h |
| Sham (no stroke) | 12.59 ± 4.54 | 10.04 ± 3.48 |
| Model (vehicle-treated) | 60.00 ± 0.00^{##} | 59.68 ± 1.10^{##} |
| Butylphthalide 10 mg/kg | 41.27 ± 21.52* | 41.92 ± 22.83* |
| ALMB-0166 15 mg/kg | 52.01 ± 13.05\hat{ } | 45.75 ± 18.89* |
| ALMB-0166 25 mg/kg | 48.52 ± 17.71* | 48.48 ± 18.26\hat{ } |
| ALMB-0166 50 mg/kg | 42.12 ± 17.16** | 42.87 ± 18.97* |

Note:
^{##}p < 0.01 vs. sham;
\hat{ }p < 0.57 vs. model;
*p < 0.05 vs. model; and
**p < 0.01 vs. model.

### Effect of Treatment with Anti-Cx43 Antibody on Grip Strength of Mice after MCAO

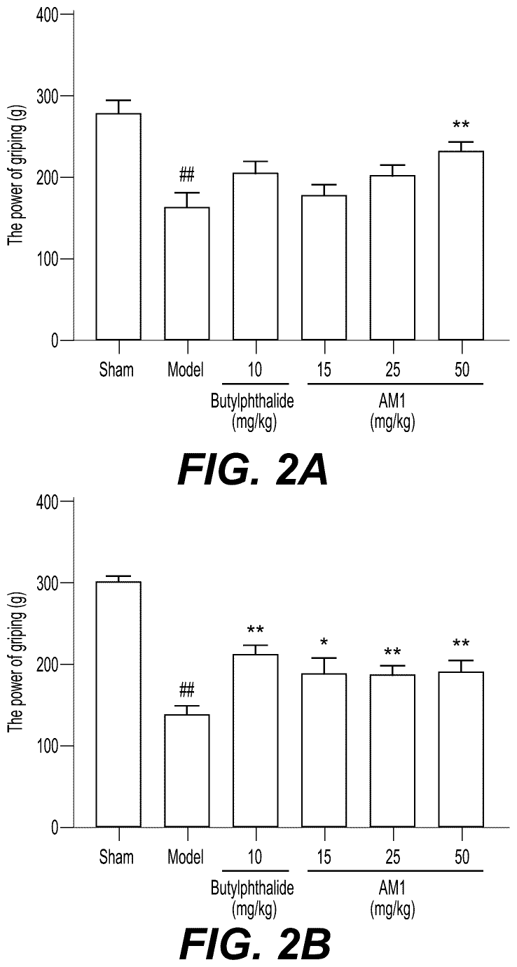

Grip strength measurements were taken as a second assessment neurological function the mouse stroke model after the stroke. The grip strength for each test group after 24- and 48-hours post-operation are shown in Table 3 and FIGS. 2A-2B. Compared with the sham test group, the model test group (vehicle-treated) showed a significantly lower grip strength at both 24- and 48-hours post-operation (p<0.01; Table 3). A significantly higher grip strength was observed for the ALMB-0166 50 mg/kg test group, but not the butylphthalide test group, as compared to the model test group at 24 hours post-procedure (p<0.01 and p=0.063, respectively; Table 3 and FIG. 2A). At 48 hours post-operation, a significantly higher grip strength was observed for the butylphthalide test group and all ALMB-0166 test groups, as compared to the model test group (Table 3 and FIG. 2B).

TABLE 3
Grip strength of animal test groups
over study period (Mean ± SEM, n = 12).

| | Grip Strength (g) | |
|---|---|---|
| Group | 24 h | 48 h |
| Sham (no stroke) | 279.60 ± 49.40 | 300.44 ± 27.68 |
| Model (vehicle-treated) | 163.11 ± 60.22^{##} | 138.12 ± 40.01^{##} |
| Butylphthalide 10 mg/kg | 206.29 ± 46.55\hat{ } | 214.71 ± 34.19** |
| ALMB-0166 15 mg/kg | 178.70 ± 44.90 | 189.95 ± 65.74* |
| ALMB-0166 25 mg/kg | 203.05 ± 45.83 | 189.96 ± 35.09** |
| ALMB-0166 50 mg/kg | 231.93 ± 42.00** | 193.00 ± 48.02** |

Note:
^{##}p < 0.01 vs. sham;
\hat{ }p = 0.63 vs. model;
*p < 0.05 vs. model; and
**p < 0.01 vs. model.

### Effect of Treatment with Anti-Cx43 Antibody on Rotarod Test Score of Mice after MCAO

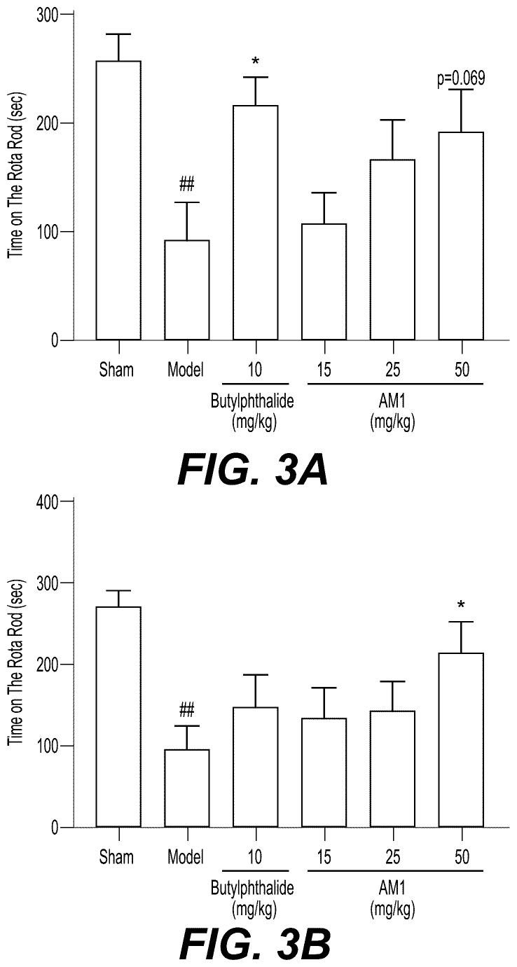

A rotarod test was performed as a third assessment of neurological function. The rotarod test scores for each test group after 24- and 48-hours post-operation are shown in Table 4 and FIGS. 3A-3B. A statistically significant difference was observed between the sham group and the model test group at both time points (p<0.01; Table 4). At 24-hours post-operation, a significantly higher rotarod test score was observed for the butylphthalide test group, as compared to the model test group (p<0.05; Table 4 and FIG. 3A). At 48-hours post-operation, a significantly higher rotarod test score was observed for the ALMB-0166 50 mg/kg test group, as compared to the model test group (p<0.05; Table 4 and FIG. 3B).

TABLE 4
Rotarod test score of animal test groups
over study period (Mean ± SEM, n = 12).

| | Time (s) | |
|---|---|---|
| Group | 24 h | 48 h |
| Sham (no stroke) | 258.42 ± 81.56 | 268.92 ± 69.31 |
| Model (vehicle-treated) | 91.58 ± 126.96^{##} | 93.83 ± 101.22^{##} |
| Butylphthalide 10 mg/kg | 216.67 ± 91.22* | 146.50 ± 137.21 |
| ALMB-0166 15 mg/kg | 106.17 ± 103.35 | 133.50 ± 127.01 |
| ALMB-0166 25 mg/kg | 167.83 ± 123.77 | 142.00 ± 119.55 |
| ALMB-0166 50 mg/kg | 192.92 ± 133.41\hat{ } | 212.00 ± 132.03* |

Note:
^{##}p < 0.01 vs. sham;
\hat{ }p < 0.69 vs. model; and
*p < 0.05 vs. Model

### Effect of Treatment with Anti-Cx43 Antibody on Cerebral Infarct Volume of Mice after MCAO

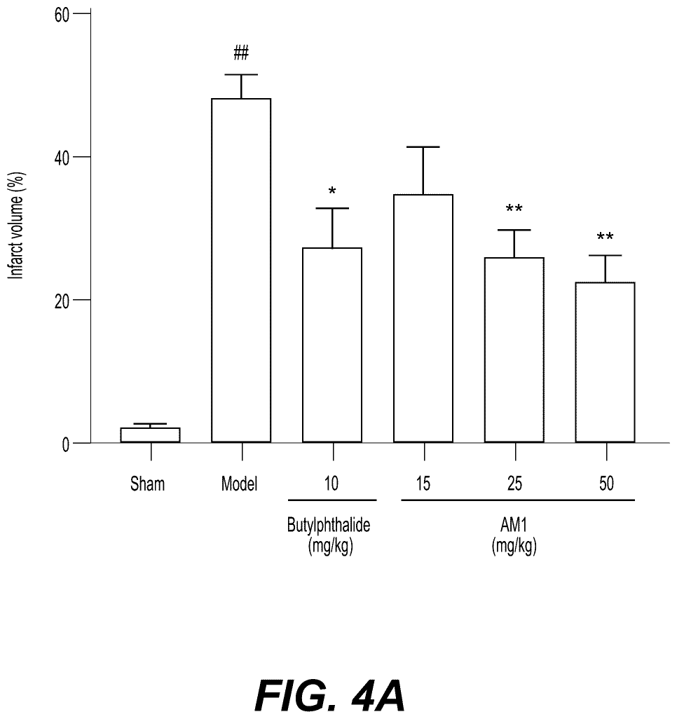

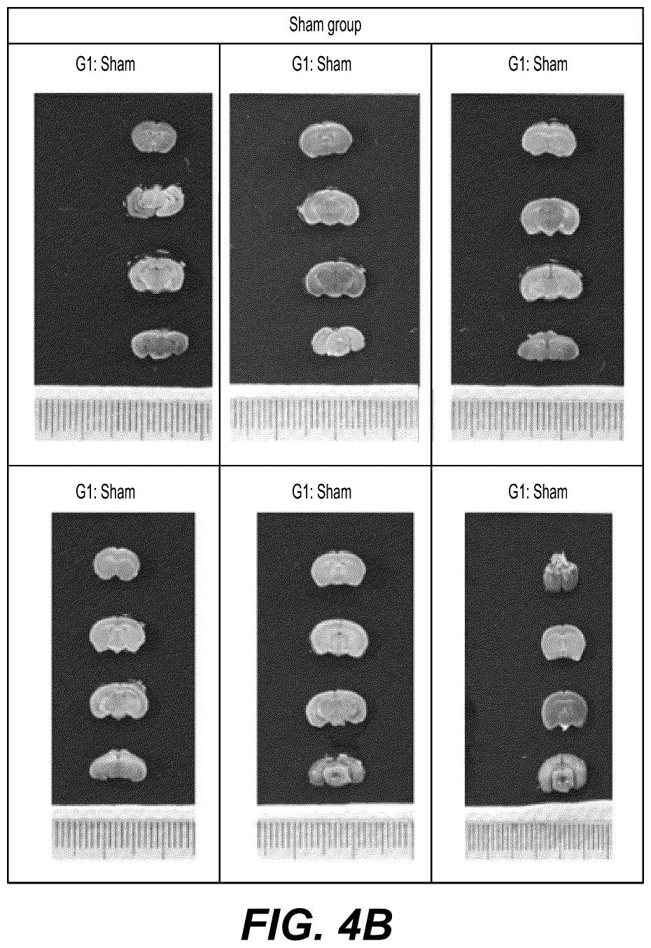

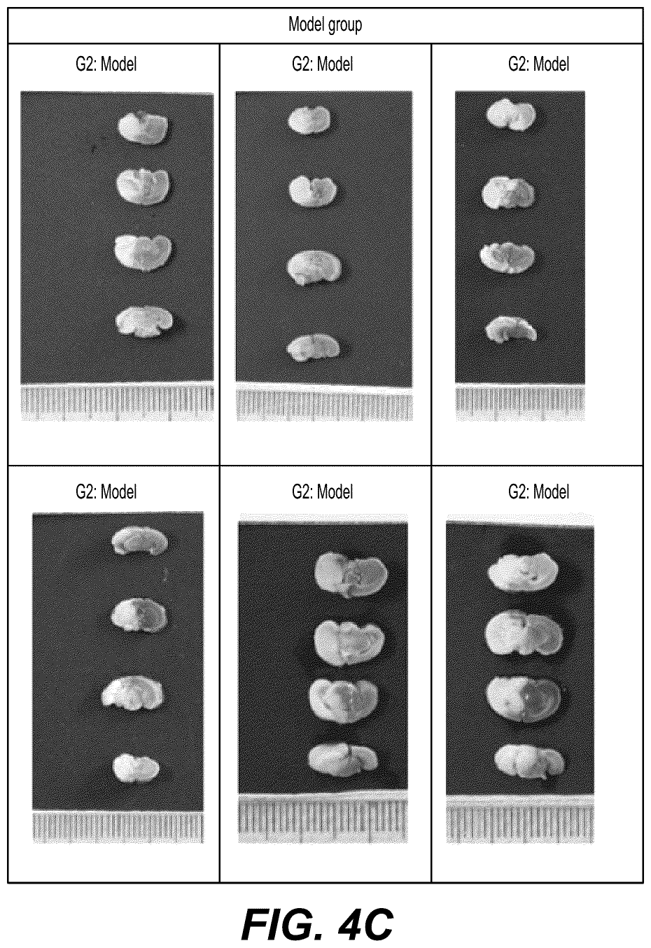

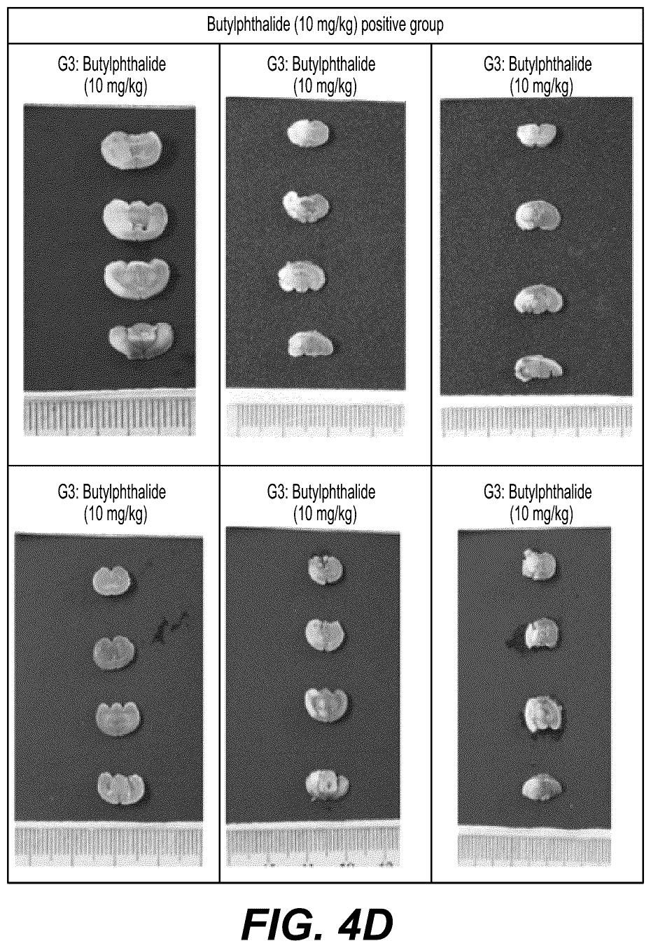

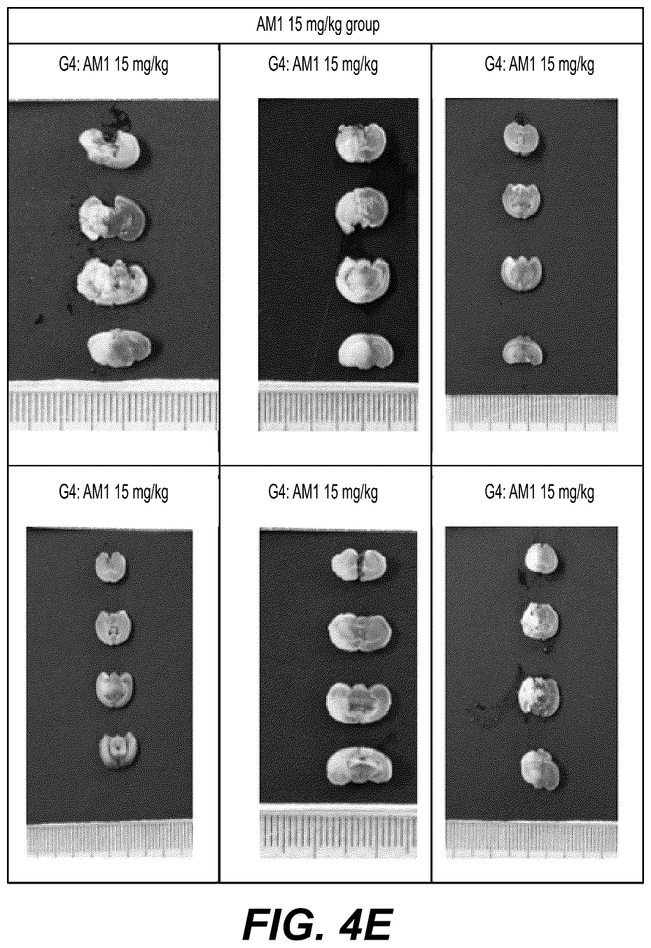

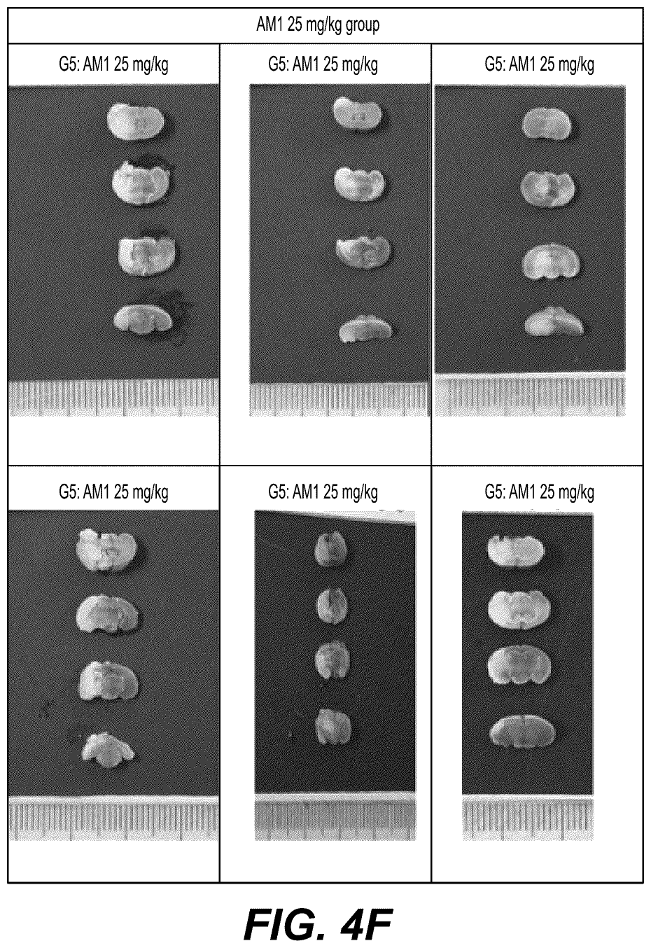

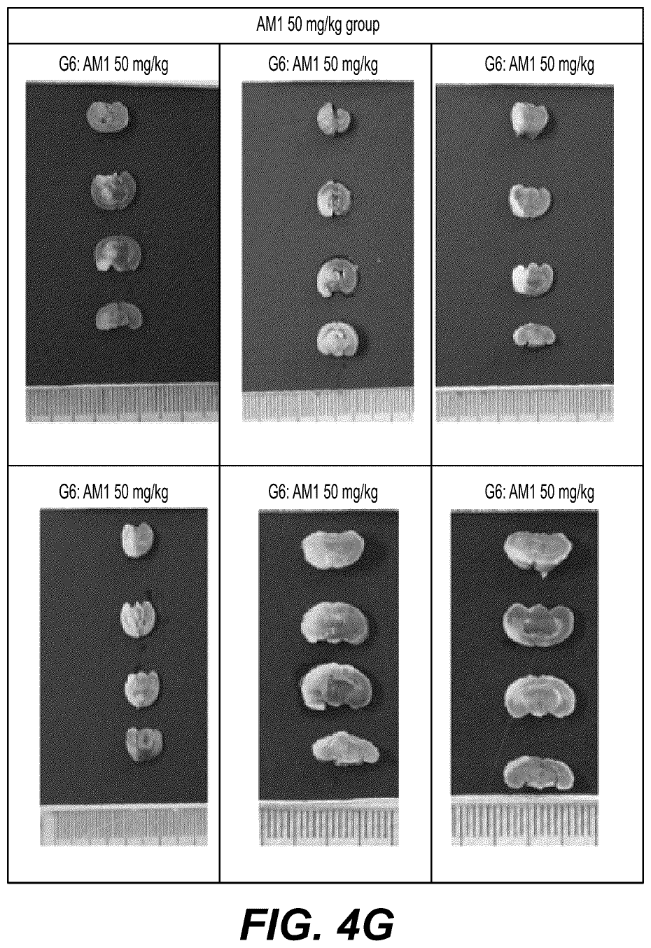

The cerebral infarct volume and the improvement on the rate of infarction of each test group was measured following 48-hours post-operation (Table 5 and FIGS. 4A-4G). A statistically significant difference in cerebral infarct volume was observed between the sham group and the model test group (p<0.01; Table 5 and FIG. 4A). The cerebral infarct volume was significantly lower in the butylphthalide test group and the ALMB-0166 25 mg/kg and 50 mg/kg test groups, as compared to the model test group (Table 5 and FIG. 4A). An improvement in the rate of infarction was observed for the butylphthalide test group as well as all ALMB-0166 test groups (Table 5).

TABLE 5
Cerebral infarct volume of animal test groups (Mean ± SEM, n = 6).

| | Cerebral Infarction | Improvement rate of |
|---|---|---|
| Group | Volume (%) | infarction (%) |
| Sham (no stroke) | 1.78 ± 1.42 | — |
| Model (vehicle-treated) | 47.90 ± 8.34^{##} | — |
| Butylphthalide 10 mg/kg | 27.29 ± 13.07* | 43.02 |
| ALMB-0166 15 mg/kg | 34.58 ± 16.15 | 27.80 |
| ALMB-0166 25 mg/kg | 25.77 ± 9.69** | 46.20 |
| ALMB-0166 50 mg/kg | 22.04 ± 9.71** | 53.98 |

Note:
^{##}p < 0.01 vs. sham;
*p < 0.05 vs. model; and
**p < 0.01 vs. model.

### Effect of Treatment with an Anti-Cx43 Antibody on Brain Water Content of Mice after MCAO

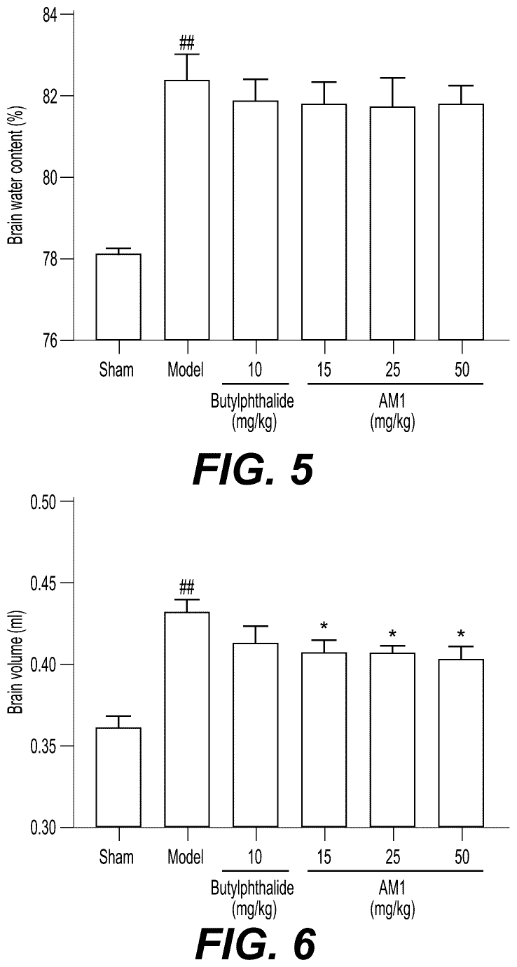

The brain water content and the brain water ratio of each test group was measured following 48-hours post-operation (Table 6 and FIG. 5). A significant difference in brain water content and brain water ratio were observed between the sham group and the model test group (p<0.01; Table 6 and FIG. 5). Compared with the model test group, no statistically significant difference was observed with all administration groups (Table 6 and FIG. 5).

TABLE 6
Brain water content of animal test groups (Mean ± SEM, n = 6).

| | Brain water content | Brain water |
|---|---|---|
| Group | (mg) | ratio (%) |
| Sham (no stroke) | 259.83 ± 9.54 | 78.18 ± 0.23 |
| Model (vehicle-treated) | 330.33 ± 25.37^{##} | 82.37 ± 1.54^{##} |
| Butylphthalide 10 mg/kg | 329.50 ± 23.60 | 81.84 ± 1.30 |
| ALMB-0166 15 mg/kg | 326.17 ± 29.81 | 81.77 ± 1.34 |
| ALMB-0166 25 mg/kg | 332.83 ± 30.01 | 81.69 ± 1.75 |
| ALMB-0166 50 mg/kg | 340.00 ± 13.01 | 81.74 ± 1.17 |

Note:
^{##}p < 0.01 vs. sham.

### Effect of Treatment with an Anti-Cx43 Antibody on Brain Volume of Mice after MCAO

The brain volume for each test group was measured following 48-hours post-operation (Table 7 and FIG. 6). A statistically significant difference in brain volume was observed between the sham group and the model test group (p<0.01; Table 7 and FIG. 6). The brain volume of mice in all ALMB-0166 test groups was significantly lower than in mice from the model test group (p<0.05; Table 7 and FIG. 6).

TABLE 7
Brain volume of animal test groups (Mean ± SEM, n = 12).

| Group | Brain volume (mL) |
|---|---|
| Sham (no stroke) | 0.362 ± 0.01 |
| Model (vehicle-treated) | 0.433 ± 0.02^{##} |
| Butylphthalide 10 mg/kg | 0.413 ± 0.03 |
| ALMB-0166 15 mg/kg | 0.408 ± 0.02* |
| ALMB-0166 25 mg/kg | 0.407 ± 0.01* |
| ALMB-0166 50 mg/kg | 0.404 ± 0.02* |

Note:
^{##}p < 0.01 vs. sham; and
*p < 0.05 vs. model.

### Conclusions

Treatment of a mouse model of stroke induced by MCAO with the anti-Cx43 antibody ALMB-0166 at dosages of 15 mg/kg, 25 mg/kg and 50 mg/kg led to improvements in neurological function post-stroke in a dose-dependent manner. Administration of ALMB-0166 also led to improvements in the cerebral infarct volume post-stroke, with a better protective rate with ALMB-0166 doses of 25 and 50 mg/kg than administration of 10 mg/kg butylphthalide. Moreover, while all doses of ALMB-0166 had a comparable effect to butylphthalide on the brain water content post-stroke, all doses of ALMB-0166 led to a higher improvement in brain volume post-stroke compared to butylphthalide.

### Example 2. Effects of Time of Treatment with an Anti-Cx43 Antibody on a Mouse Model of Stroke

This Example describes the evaluation of the effects of administration time of an anti-Cx43 antibody in the treatment of stroke in a mouse model of stroke.

### Study Objective

The purpose of the study was to test the effect of the administration time of the anti-Cx43 antibody ALMB-0166 on stroke symptoms in a mouse model of stroke induced by middle cerebral artery occlusion (MCAO).

### Regulatory Compliance

The experiment complied with Standard Operating Procedures (SOPs).

### Materials and Methods

### Test Materials

The anti-Cx43 antibody used in the study was ALMB-0166 (AlaMab Therapeutics Inc.). Butylphthalide and sodium chloride (CSPC NBP Pharmaceutical Group Co., Ltd.) was used as a positive control. Other materials used in the study included 0.9% normal saline (Anhui Shuanghe Pharmaceutical Co., Ltd). sterile water for injection (Sichuan Kelun Pharmaceutical Co., Ltd.), isoflurane (RWD Life Science Inc.), phosphate buffered saline (Thermo, batch no.), 2,3,5-triphenyltetrazolium chloride (TTC; Bide Pharmaceutical Technology, Co., Ltd.), and a 40% formaldehyde solution (Sinopharm Chemical Reagent Co., Ltd.)

For animal dosing, solutions of ALMB-0166 at 1 mg/mL, 2.5 mg/mL and 5 mg/mL were prepared by diluting the antibody stock solution with PBS. A 1% w/v TTC solution was also prepared for the study.

### Equipment

Equipment used in this study included an inhalation anesthesia apparatus (Midmark Corporation, type VWR), a mouse thread bolt (Beijing Cinontech Co., Ltd., type A4-162450), and a mouse brain mold (RWD Life Sciences Inc., type 175-800).

### Animals and Husbandry

Male Institute of Cancer Research (ICR) mouse (Specific Pathogen Free grade; Shanghai Bikai Keyi Biotechnology Co. Ltd) were obtained in the study. Each mouse was weight at the start of treatment, with the average weight ranging from 25-30 grams. Upon arrival at the test facility, the animals were housed at five mice per cage and acclimated for three to five days.

Animals were kept in an environment set to maintain a temperature of 23±2° C., with humidity of 40-70%, and a 12-hour light/12-hour dark cycle. The 12-hour dark cycle was temporarily interrupted to accommodate study procedures. SPF Rat Growth Breeding Feed (Beijing Keao Xieli Feed Co., Ltd)) was provided ad libitum throughout the in-life part of the study. Reverse Osmosis water was available ad libitum.

The animals used in the study were selected based on overall health and acclimation to caging. The mice were fasted for 12 hours before experimental modeling and were free to access both food and water afterwards.

A total of 96 rats were selected for participation in the study. Studies using common mammalian laboratory animals such as mice, dogs, and monkeys are essential and routinely used for the evaluation of the pharmacokinetics, pharmacodynamics and toxicology characteristics of new chemical entities. The number of animals in each test group was the minimal number needed for the assessment of variability between test animals.

### Study Design

### Animal Groups and Drugs

For the study, animal groups were formed as indicated in Table 8 below. The administration of 10 mg/kg butylphthalide and 50 mg/kg ALMB-0166 was started 30 minutes, 2 hours, or 6 hours after the MCAO procedure of the mice.

TABLE 8
Treatment and dosing information for animal test groups.

| | | | | | | Dosing route, |
|---|---|---|---|---|---|---|
| | | Animal | Dose | Dose volume | Concentration | frequency, and |
| Group | Treatment | Number | (mg/kg) | (mL/kg) | (mg/mL) | period |
| 1 | Sham (no stroke) | 12 | — | — | — | — |
| 2 | Model (vehicle- | 12 | — | 10 | — | Intravenous |
| | treated) | | | | | (i.v.), once |
| 3 | Butylphthalide - 0.5 hr | 12 | 10 | 20 | 0.25 | Intravenous |
| | | | | | | (i.v.), once |
| 4 | Butylphthalide - 2 hr | 12 | 10 | 20 | 0.25 | Intravenous |
| | | | | | | (i.v.), once |
| 5 | Butylphthalide - 6 hr | 12 | 10 | 20 | 0.25 | Intravenous |
| | | | | | | (i.v.), once |
| 6 | ALMB-0166 - 0.5 hr | 12 | 50 | 10 | 5 | Intravenous |
| | | | | | | (i.v.), once |
| 7 | ALMB-0166 - 2 hr | 12 | 50 | 10 | 5 | Intravenous |
| | | | | | | (i.v.), once |
| 8 | ALMB-0166 - 6 hr | 12 | 50 | 10 | 5 | Intravenous |
| | | | | | | (i.v.), once |

### Models and Methods

Ninety-six (96) ICR mice received behavioral training after adaptive feeding period and before the operation to establish stroke models. The behavioral training included beam-walking and rotarod test. The experimental mice were then randomly divided into 8 groups according to the values of the body weight, with 12 mice in each test group as described in Table 1.

The mice were fasted overnight before modeling and were anesthetized with isoflurane. Right middle cerebral artery occlusion (MCAO) was induced in mice using the intraluminal filament technique to mimic focal cerebral ischemic stroke. After 1 hr, reperfusion was allowed by removing the occlusion.

Administration of each treatment was performed 0.5 hrs, 2 hrs, or 6 hrs after the infarction. The neurological function score was evaluated at 24 hrs and 48 hrs after reperfusion, as was assessed by beam-walking, grip strength, and rotarod tests. At 48 hrs after reperfusion, the whole brain tissue of all rats was harvested to assess the cerebral infarct area by TTC staining, as well as to measure brain volume and brain water content, two indicators of acute cerebral edema.

### Statistical Analysis

The neurological function scores, the cerebral infarct volume, the brain volume, and the brain water content were expressed as the mean and standard error (Mean±SEM). The data between model group and administration (treatment) group at multiple time points were analyzed by repeated measures analysis of variance method with SPSS Statistics 21 software, and Tamhane's method for subsequent analysis. p<0.05 was considered to have a significant difference, and p<0.01 was considered to have a very significant difference. The final data were plotted with GraphPad Prism 6 software.

### Results

General Clinical Symptoms of Mice after Administration

All animals had no visible abnormalities during the experimental period.

### Effect of Time of Treatment with Anti-Cx43 Antibody on Beam-Walking of Mice after MCAO

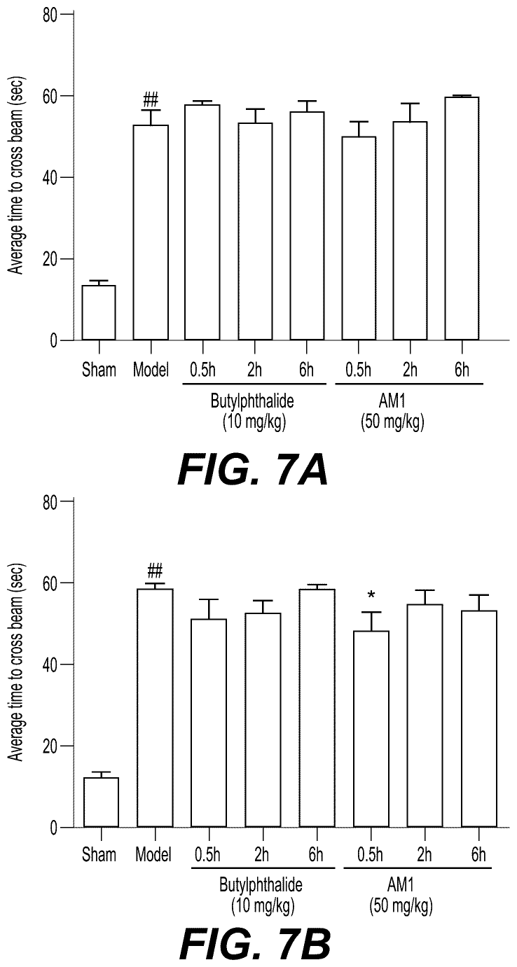

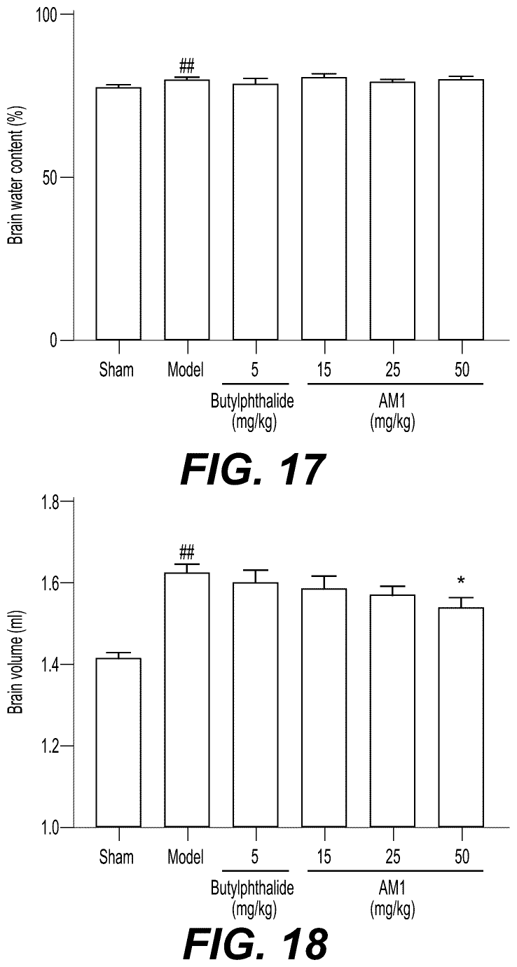

Beam-walking test was used to assess neurological function the mouse stroke model after the stroke. The beam-walking time of each test group after 24- and 48-hours post-operation are shown in Table 9 and FIGS. 17A-7B. A statistical difference was observed in the beam-walking time of the model (vehicle-treated) test group as compared to the sham group at both 24- and 48-hours post-operation (p<0.01; Table 9). At 24 hours post-operation, no significant difference in beam walking time was observed between the the butylphthalide and the ALMB-0166 test groups and the model test group (Table 9 and FIG. 7A). At 48 hours post-operation, a significantly lower beam walking time was observed for the ALMB-0166 30-minute administration group, as compared to the model test group (p<0.05; Table 9 and FIG. 7B), while no significant difference was observed between the model group and the ALMB-0166 2-hr and 6-hr administration groups or between the model group and all butylphthalide test groups.

TABLE 9
Beam-walking time of animal test groups
over study period (Mean ± SEM, n = 12)

| | Time (s) | |
|---|---|---|
| Group | 24 h | 48 h |
| Sham (no stroke) | 13.50 ± 4.22 | 12.31 ± 3.49 |
| Model (vehicle-treated) | 52.78 ± 12.21^{##} | 58.74 ± 4.37^{##} |
| Butylphthalide—0.5 hr | 57.69 ± 3.94 | 51.37 ± 16.46 |
| Butylphthalide—2 hr | 53.12 ± 12.48 | 52.73 ± 10.98 |
| Butylphthalide—6 hr | 10.32 | 58.57 ± 3.23 |
| ALMB-0166—0.5 hr | 49.86 ± 12.61 | 48.25 ± 16.31* |
| ALMB-0166—2 hr | 53.72 ± 14.90 | 55.22 ± 11.48 |
| ALMB-0166—6 hr | 59.67 ± 1.14 | 53.26 ± 13.26 |

Note:
^{##}p < 0.01 vs. sham; and
*p < 0.05 vs. model.

### Effect of Time of Treatment with Anti-Cx43 Antibody on Grip Strength of Mice after MCAO

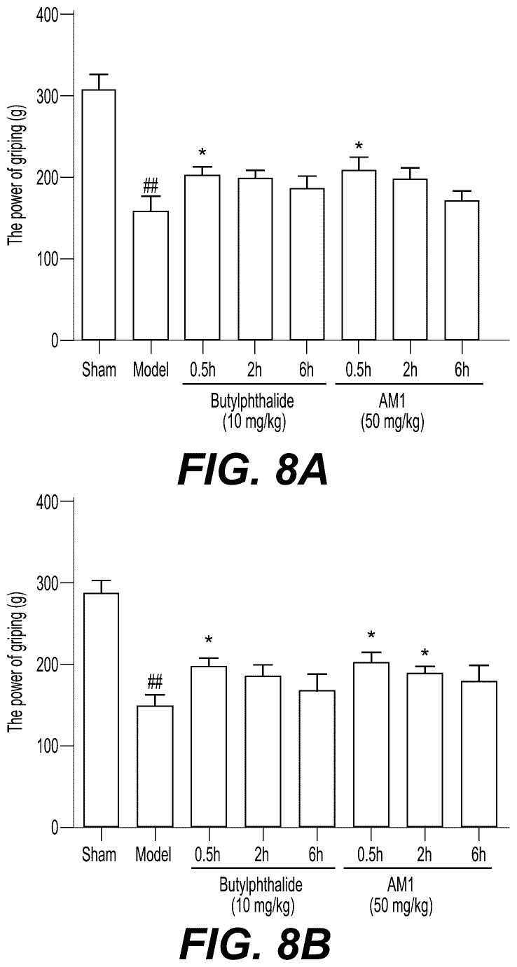

Grip strength measurements were taken as a second assessment neurological function the mouse stroke model after the stroke. The grip strength for each test group after 24- and 48-hours post-operation are shown in Table 10 and FIGS. 8A-8B. Compared with the sham test group, the model test group (vehicle-treated) showed a significantly lower grip strength at both 24- and 48-hours post-operation (p<0.01; Table 10). A significantly higher grip strength was observed for the butylphthalide and ALMB-0166 30-minute administration groups, as compared to the model test group at 24 hours post-procedure (p<0.05; Table 10 and FIG. 8A). At 48 hours post-operation, a significantly higher grip strength was observed for the butylphthalide test group and the ALMB-0166 30-minute and 2-hr administration groups, as compared to the model test group (p<0.05; Table 10 and FIG. 8B).

TABLE 10
Grip strength of animal test groups
over study period (Mean ± SEM, n = 12).

| | Grip Strength (g) | |
|---|---|---|
| Group | 24 h | 48 h |
| Sham (no stroke) | 308.90 ± 60.35 | 286.63 ± 54.59 |
| Model (vehicle-treated) | 159.73 ± 60.12^{##} | 148.71 ± 47.43^{##} |
| Butylphthalide—0.5 hr | 203.13 ± 35.30* | 196.04 ± 41.72* |
| Butylphthalide—2 hr | 199.41 ± 38.48 | 186.33 ± 44.64 |
| Butylphthalide—6 hr | 187.08 ± 53.81 | 168.16 ± 66.94 |
| ALMB-0166—0.5 hr | 211.41 ± 47.85* | 201.12 ± 47.31* |
| ALMB-0166—2 hr | 199.18 ± 45.89 | 189.23 ± 28.49* |
| ALMB-0166—6 hr | 171.16 ± 46.47 | 180.33 ± 62.44 |

Note:
^{##}p < 0.01 vs. sham; and
*p < 0.05 vs. model. model.

### Effect of Time of Treatment with Anti-Cx43 Antibody on Rotarod Test Score of Mice after MCAO

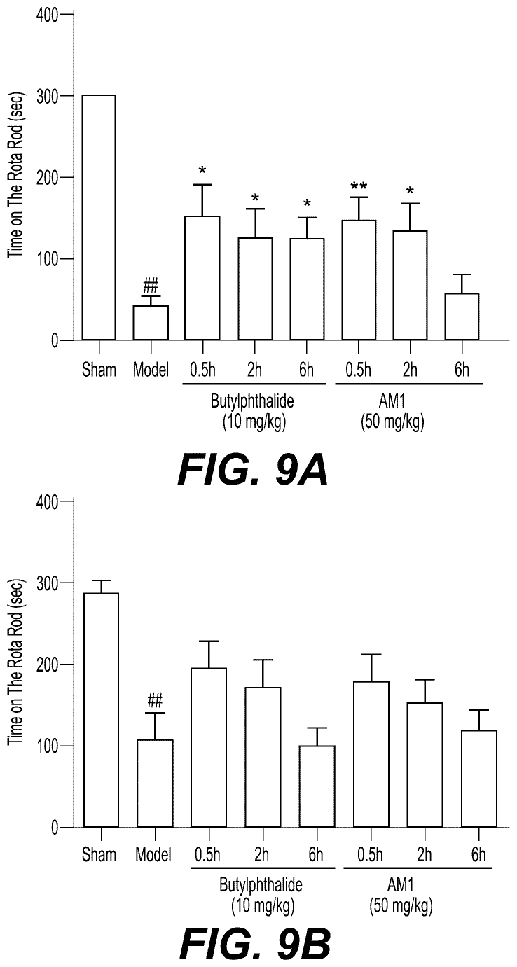

A rotarod test was performed as a third assessment of neurological function. The rotarod test scores for each test group after 24- and 48-hours post-operation are shown in Table 11 and FIGS. 9A-9B. A statistically significant difference was observed between the sham group and the model test group at both time points (p<0.01; Table 11). At 24-hours post-operation, a significantly higher rotarod test score was observed for all butylphthalide test groups, as well as the ALMB-0166 30-minute and 2-hr administration groups, as compared to the model test group (Table 11 and FIG. 9A). At 48-hours post-operation, no significant difference in rotarod test score was observed for all butylphthalide and ALMB-0166 test groups compared to the model test group (Table 11 and FIG. 9B).

TABLE 11
Rotarod test score of animal test groups
over study period (Mean ± SEM, n = 12).

| | Time (s) | | | |
|---|---|---|---|---|
| Group | 24 h | | 48 h | |
| Sham (no stroke) | 300.00 ± | 0.00 | 284.42 ± | 45.36 |
| Model (vehicle-treated) | 42.00 ± | 42.46^{##} | 104.92 ± | 123.13^{##} |
| Butylphthalide—0.5 hr | 151.83 ± | 133.78* | 193.25 ± | 125.61 |
| Butylphthalide—2 hr | 124.67 ± | 120.08* | 171.67 ± | 119.90 |
| Butylphthalide—6 hr | 121.42 ± | 105.31* | 98.00 ± | 84.79 |
| ALMB-0166—0.5 hr | 145.50 ± | 101.07** | 178.42 ± | 114.72 |
| ALMB-0166—2 hr | 133.50 ± | 116.4⁸* | 150.67 ± | 109.13 |
| ALMB-0166—6 hr | 56.67 ± | 79.42 | 116.33 ± | 93.62 |

Note:
^{##}p < 0.01 vs. sham;
*p < 0.05 vs. model; and
**p < 0.01 vs. model.

### Effect of Time of Treatment with Anti-Cx43 Antibody on Cerebral Infarct Volume of Mice after MCAO

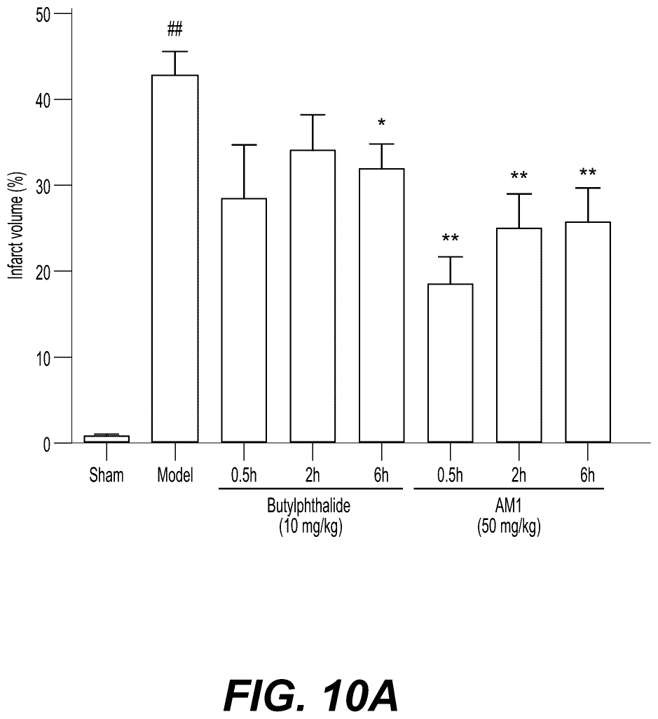

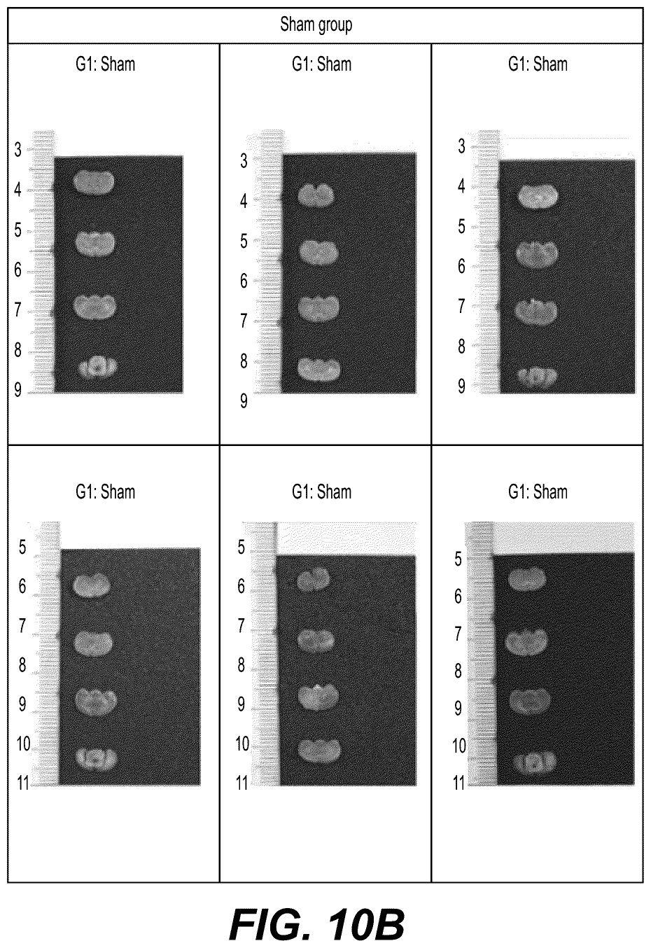

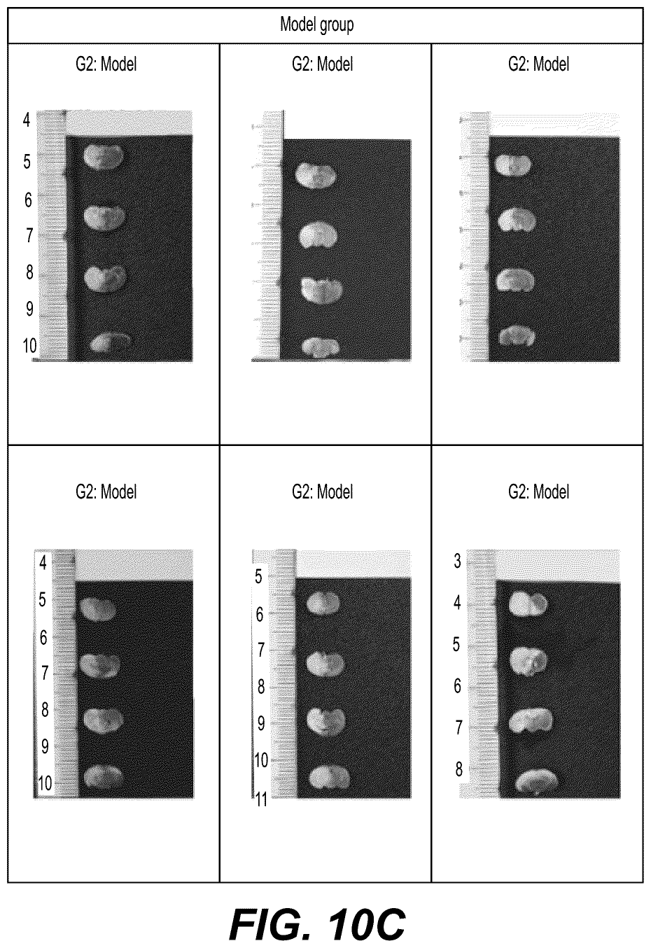

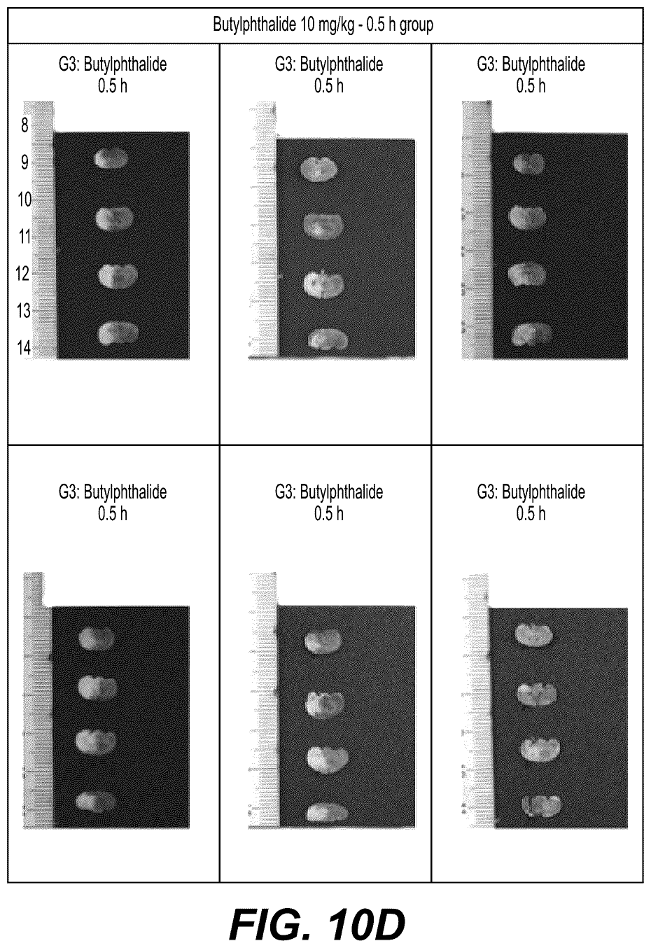

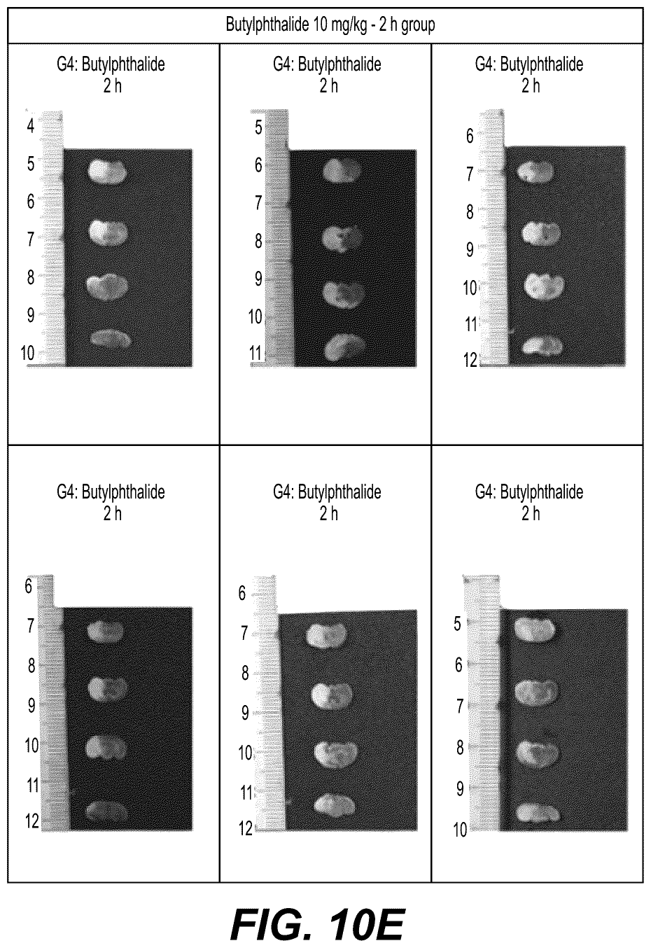

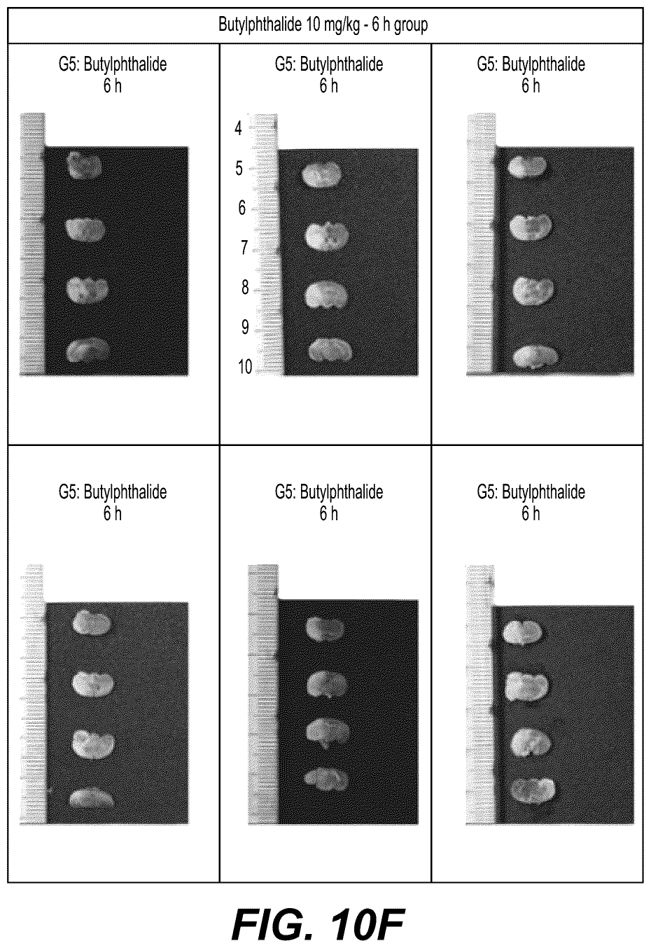

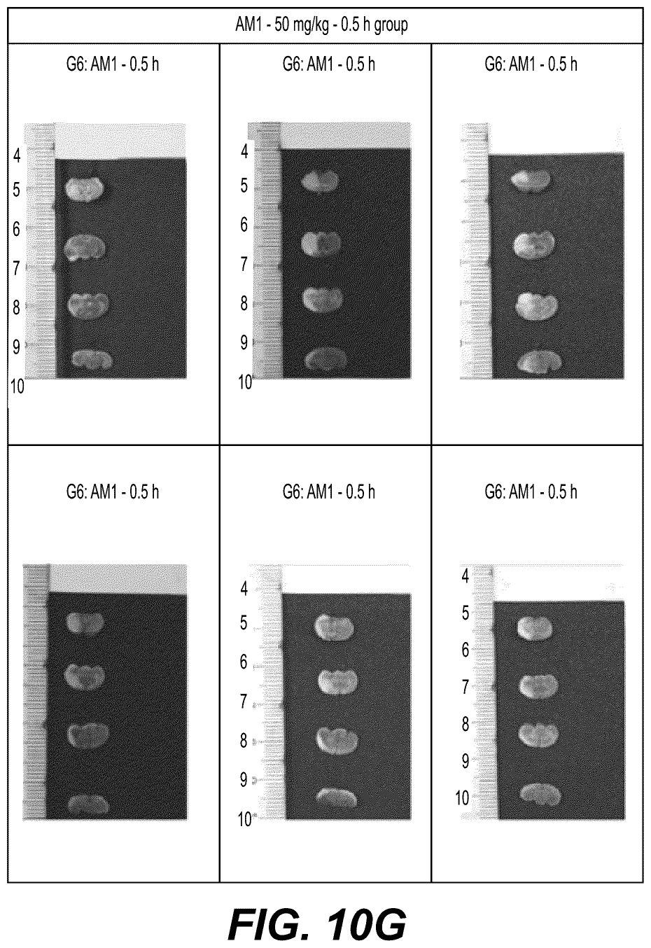

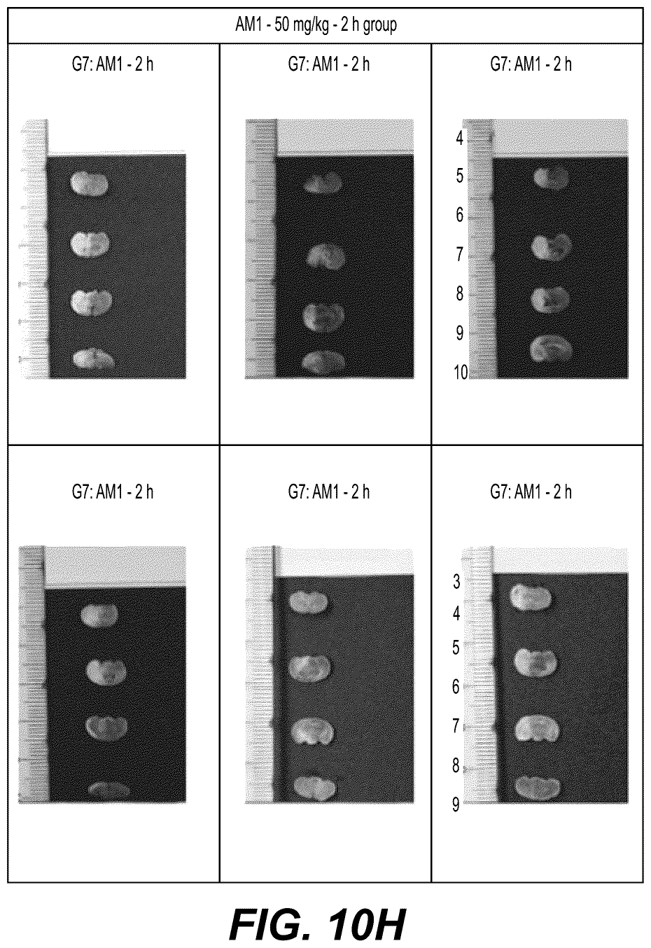

The cerebral infarct volume and the improvement on the rate of infarction of each test group was measured following 48-hours post-operation (Table 12 and FIGS. 10A-10I). A statistically significant difference in cerebral infarct volume was observed between the sham group and the model test group (p<0.01; Table 12 and FIG. 10A). The cerebral infarct volume was significantly lower in the butylphthalide 6-hr administration group, as well as in all ALMB-0166 test groups, as compared to the model test group (Table 12 and FIG. 10A). An improvement in the rate of infarction was observed for all butylphthalide and ALMB-0166 test groups (Table 12).

TABLE 12
Cerebral infarct volume of
animal test groups (Mean ± SEM, n = 6)

| | Cerebral | | Improvement |
|---|---|---|---|
| | Infarction | | rate of |
| Group | Volume (%) | | infarction (%) |
| Sham (no stroke) | 0.73 ± | 0.45 | — |
| Model (vehicle-treated) | 42.95 ± | 6.93^{##} | — |
| Butylphthalide—0.5 hr | 28.56 ± | 15.23\hat{ } | 33.50 |
| Butylphthalide—2 hr | 34.25 ± | 9.70 | 20.25 |
| Butylphthalide—6 hr | 31.89 ± | 7.29* | 25.76 |
| ALMB-0166—0.5 hr | 18.54 ± | 7.46** | 56.83 |
| ALMB-0166—2 hr | 25.16 ± | 9.67** | 41.42 |
| ALMB-0166—6 hr | 25.79 ± | 9.81** | 39.96 |

Note:
^{##}p < 0.01 vs. sham;
\hat{ }p = 0.073;
*p < 0.05 vs. model; and
**p < 0.01 vs. model.

### Effect of Time of Treatment with Anti-Cx43 Antibody on Brain Water Content of Mice after MCAO

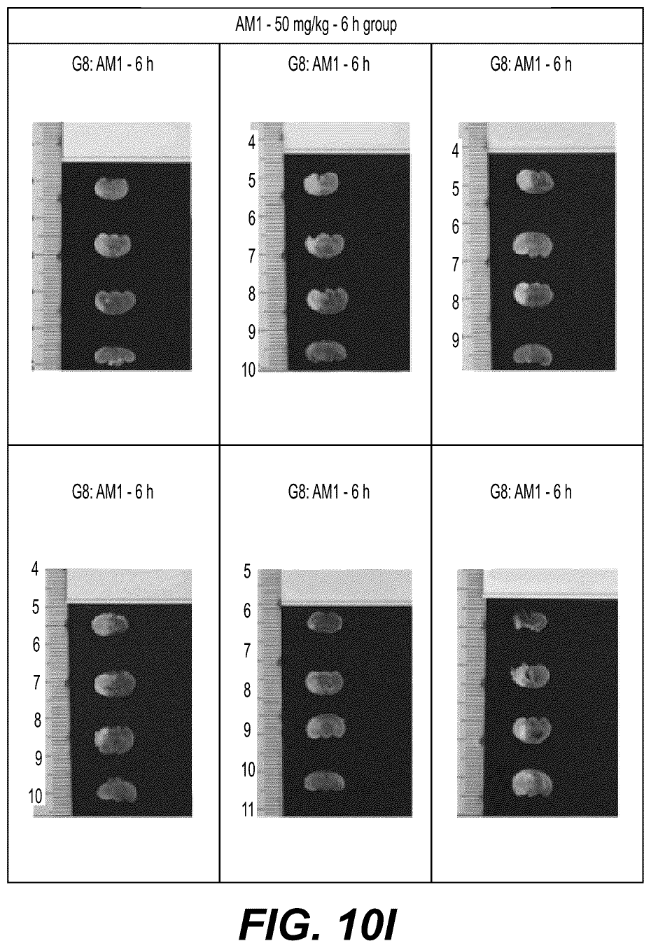

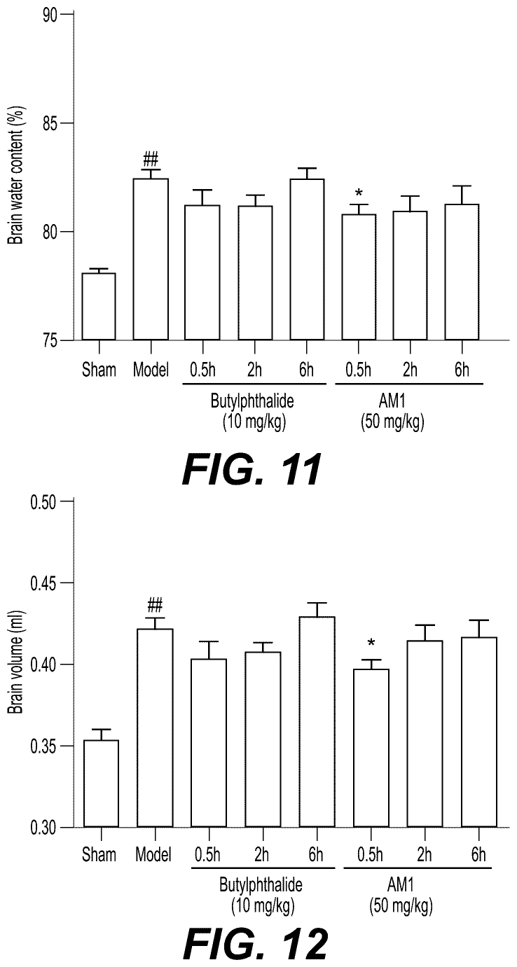

The brain water content and the brain water ratio of each test group was measured following 48-hours post-operation (Table 13 and FIG. 11). A significant difference in brain water content and brain water ratio were observed between the sham group and the model test group (p<0.01; Table 13 and FIG. 11). The butylphthalide 30-minute and 2-hr administration group and all ALMB-0166 administrations groups showed a lower brain water content as compared to the model test group (Table 13 and FIG. 11). The ALMB-0166 30-minute administration group showed a significant decrease in the brain water content (p<0.05; Table 13).

TABLE 13
Brain water content of animal test groups (Mean ± SEM, n = 6).

| | Brain water | | Brain water | |
|---|---|---|---|---|
| Group | content (mg) | | ratio (%) | |
| Sham (no stroke) | 275.83 ± | 15.68 | 78.09 ± | 0.57 |
| Model (vehicle-treated) | 341.83 ± | 22.92^{##} | 82.47 ± | 0.85^{##} |
| Butylphthalide—0.5 hr | 329.00 ± | 34.59 | 81.23 ± | 1.69 |
| Butylphthalide—2 hr | 326.33 ± | 14.35 | 81.25 ± | 1.06 |
| Butylphthalide—6 hr | 342.83 ± | 27.80 | 82.36 ± | 1.22 |
| ALMB-0166—0.5 hr | 316.33 ± | 22.93 | 80.88 ± | 0.89* |
| ALMB-0166—2 hr | 319.67 ± | 33.69 | 80.92 ± | 1.78 |
| ALMB-0166—6 hr | 328.67 ± | 34.30 | 81.30 ± | 1.82 |

Note:
^{##}p < 0.01 vs. sham; and
*p < 0.05 vs. model.

### Effect of Time of Treatment Anti-Cx43 Antibody on Brain Volume of Mice after MCAO

The brain volume for each test group was measured following 48-hours post-operation (Table 14 and FIG. 12). A statistically significant difference in brain volume was observed between the sham group and the model test group (p<0.01; Table 14 and FIG. 12). A significantly lower brain volume was observed for the ALMB-0166 30-minute administration group as compared to the model test group (p<0.05; Table 14 and FIG. 12).

TABLE 14
Brain volume of animal test groups (Mean ± SEM, n = 12).

| Group | Brain volume (mL) |
|---|---|
| Sham (no stroke) | 0.353 ± 0.02 |
| Model (vehicle-treated) | 0.421 ± 0.02^{##} |
| Butylphthalide—0.5 hr | 0.403 ± 0.04 |
| Butylphthalide—2 hr | 0.407 ± 0.02 |
| Butylphthalide—6 hr | 0.427 ± 0.03 |
| ALMB-0166—0.5 hr | 0.397 ± 0.02* |
| ALMB-0166—2 hr | 0.414 ± 0.03 |
| ALMB-0166—6 hr | 0.416 ± 0.04 |

Note:
^{##}p < 0.01 vs. sham; and
*p < 0.05 vs. model.

### Conclusions

Treatment of a mouse model of stroke induced by MCAO with the anti-Cx43 antibody ALMB-0166 led to improvements in neurological function post-stroke regardless of time of administration of the antibody. While administration of ALMB-0166 led to improvements in cerebral infarct volume, brain water content and brain volume, administration of ALMB-0166 30 minutes post-stroke resulted in the best protective effect following stroke.

### Example 3. Effects of Treatment with an Anti-Cx43 Antibody on a Rat Model of Stroke

This Example describes the evaluation of an anti-Cx43 antibody for the treatment of stroke in a rat model of stroke.

### Study Objective

The purpose of the study was to test the effect of the anti-Cx43 antibody ALMB-0166 on stroke symptoms in a rat model of stroke induced by middle cerebral artery occlusion (MCAO).

### Regulatory Compliance

The experiment complied with Standard Operating Procedures (SOPs).

### Materials and Methods

### Test Materials

The anti-Cx43 antibody used in the study was ALMB-0166 (AlaMab Therapeutics Inc.). Butylphthalide and sodium chloride (CSPC NBP Pharmaceutical Group Co., Ltd.) was used as a positive control. Other materials used in the study included 0.9% normal saline (Anhui Shuanghe Pharmaceutical Co., Ltd), sterile water for injection (Sichuan Kelun Pharmaceutical Co., Ltd.), isoflurane (RWD Life Science Inc.), phosphate buffered saline (Thermo, batch no.), 2,3,5-triphenyltetrazolium chloride (TTC; Bide Pharmaceutical Technology, Co., Ltd.), and a 40% formaldehyde solution (Sinopharm Chemical Reagent Co., Ltd.)

For animal dosing, solutions of ALMB-0166 at 1 mg/mL, 2.5 mg/mL and 5 mg/mL were prepared by diluting the antibody stock solution with PBS. A 1% w/v TTC solution was also prepared for the study.

### Equipment

Equipment used in this study included an inhalation anesthesia apparatus (Midmark Corporation, type VWR), a mouse thread bolt (Guangzhou Jialing Biotechnology Co., Ltd., type L3400), and a rat brain mold (RWD Life Sciences Inc., type 175-300).

### Animals and Husbandry

Male Sprague Dawley (SD) rats (Specific Pathogen Free grade; Shanghai Slac Laboratory Animal Co. Ltd) were obtained in the study. Each rat was weight at the start of treatment, with the average weight ranging from 180-200 grams. Upon arrival at the test facility, the animals were housed at five rats per cage and acclimated for one to three days.

Animals were kept in an environment set to maintain a temperature of 23±2° C., with humidity of 40-70%, and a 12-hour light/12-hour dark cycle. The 12-hour dark cycle was temporarily interrupted to accommodate study procedures. SPF Rat Growth Breeding Feed (Beijing Keao Xieli Feed Co., Ltd)) was provided ad libitum throughout the in-life part of the study. Reverse Osmosis water was available ad libitum.

The animals used in the study were selected based on overall health and acclimation to caging. The rats were fasted for 12 hours before experimental modeling and were free to access both food and water afterwards.

A total of 72 rats were selected for participation in the study. Studies using common mammalian laboratory animals such as rats, dogs, and monkeys are essential and routinely used for the evaluation of the pharmacokinetics, pharmacodynamics and toxicology characteristics of new chemical entities. The number of animals in each test group was the minimal number needed for the assessment of variability between test animals.

### Study Design

### Animal Groups and Drugs

For the study, animal groups were formed as indicated in Table 15 below. The administration of the respective treatments was started 30 minutes after the MCAO procedure of the rats.

TABLE 15
Treatment and dosing information for animal test groups.

| | | | | | | Dosing route, |
|---|---|---|---|---|---|---|
| | | Animal | Dose | Dose volume | Concentration | frequency, and |
| Group | Treatment | Number | (mg/kg) | (mL/kg) | (mg/mL) | period |
| 1 | Sham (no stroke) | 12 | — | — | — | — |
| 2 | Model (vehicle- | 12 | — | 10 | 1 | Intravenous |
| | treated) | | | | | (i.v.), once |
| 3 | Butylphthalide 5 | 12 | 5 | 20 | 0.25 | Intravenous |
| | mg/kg | | | | | (i.v.), twice |
| | | | | | | daily (Bid) |
| 4 | ALMB-0166 15 | 12 | 15 | 10 | 1.5 | Intravenous |
| | mg/kg | | | | | (i.v.), once |
| 5 | ALMB-0166 25 | 12 | 25 | 10 | 2.5 | Intravenous |
| | mg/kg | | | | | (i.v.), once |
| 6 | ALMB-0166 50 | 12 | 50 | 10 | 5 | Intravenous |
| | mg/kg | | | | | (i.v.), once |

### Models and Methods

Seventy-two (72) SD rats received behavioral training after adaptive feeding period and before the operation to establish stroke models. The behavioral training included beam-walking, grip and rotarod tests. The experimental rats were then randomly divided into 6 groups according to the body weight, with 12 rats in each test group as described in Table 15.

The rats were fasted overnight before modeling and were anesthetized with isoflurane. Right middle cerebral artery occlusion (MCAO) was induced in each rat using the intraluminal filament technique to mimic focal cerebral ischemic stroke. After 2 hrs, reperfusion was allowed by removing the occlusion.

Administration of each treatment was performed 0.5 h after the infarction. The rats were scored by the Longa method at 24-hours post-procedure. The neurological function was evaluated at 24-hr and 48-hr after reperfusion, and was assessed by beam-walking, grip strength, and rotarod tests. At 48 h after reperfusion, the whole brain tissue of all rats was harvested to assess the cerebral infarct area by TTC staining, as well as to measure brain volume and brain water content, two indicators of acute cerebral edema.

### Statistical Analysis

The neurological function scores, the cerebral infarct volume, the brain volume, and the brain water content were expressed as the mean and standard error (Mean±SEM). The data between model group and administration (treatment) group at multiple time points were analyzed by repeated measures analysis of variance method with SPSS Statistics 21 software, and Tamhane's method for subsequent analysis. p<0.05 was considered to have a significant difference, and p<0.01 was considered to have a very significant difference. The final data were plotted with GraphPad Prism 6 software.

### Results

General Clinical Symptoms of Rats after Administration

All animals had no visible abnormalities during the experimental period.

### Effect of Treatment with Anti-Cx43 Antibody on Beam-Walking of Rats after MCAO

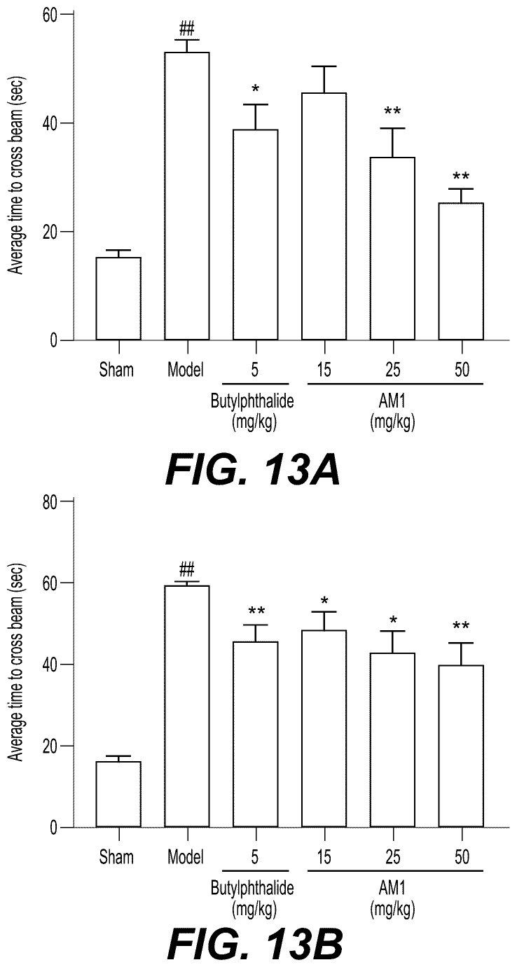

Beam-walking test was used to assess neurological function the rat stroke model after the stroke. The beam-walking time of each test group after 24- and 48-hours post-operation are shown in Table 16 and FIGS. 13A-13B. A statistical difference was observed in the beam-walking time of the model (vehicle-treated) test group as compared to the sham group at both 24- and 48-hours post-operation (p<0.01; Table 16). At 24 hours post-operation, the ALMB-0166 25 mg/kg and 50 mg/kg groups showed a significantly lower beam walking time as compared to the model test group (p<0.01; Table 16 and FIG. 13A). At 48 hours post-operation, a significantly lower beam walking time was observed for all butylphthalide and ALMB-0166 test groups as compared to the model test group (Table 16 and FIG. 13B).

TABLE 16
Beam-walking time of animal test groups
over study period (Mean ± SEM, n = 12)

| | Time (s) | | | |
|---|---|---|---|---|
| Group | 24 h | | 48 h | |
| Sham (no stroke) | 15.02 ± | 5.01 | 15.86 ± | 5.38 |
| Model (vehicle-treated) | 52.92 ± | 8.61^{##} | 59.48 ± | 1.82^{##} |
| Butylphthalide 10 mg/kg | 38.91 ± | 15.66* | 45.80 ± | 13.68** |
| ALMB-0166 15 mg/kg | 46.73 ± | 15.93 | 49.44 ± | 14.51* |
| ALMB-0166 25 mg/kg | 33.68 ± | 17.70** | 42.58 ± | 19.19* |
| ALMB-0166 50 mg/kg | 25.27 ± | 9.03** | 39.84 ± | 19.55** |

Note:
^{##}p < 0.01 vs. sham;
*p < 0.05 vs. model; and
**p < 0.01 vs. model.

### Effect of Treatment with Anti-Cx43 Antibody on Grip Strength of Rats after MCAO

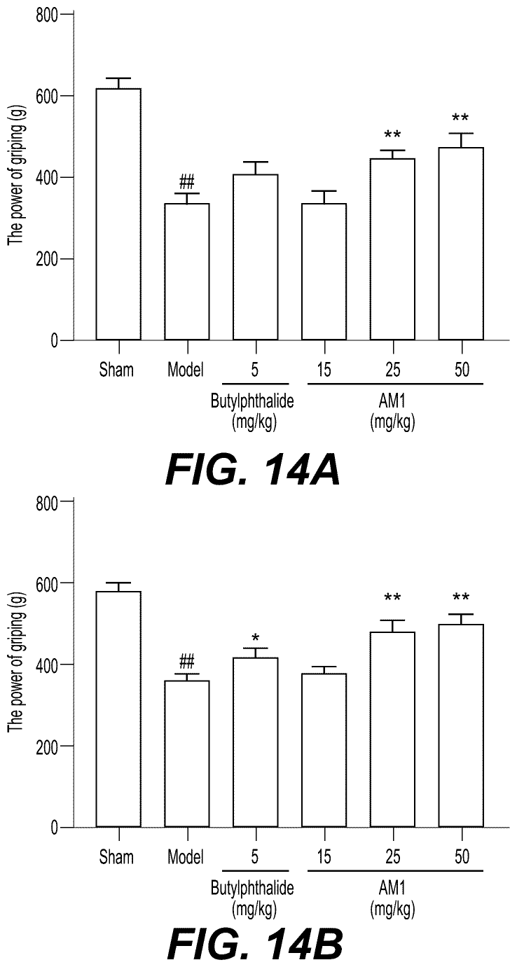

Grip strength measurements were taken as a second assessment neurological function the rat stroke model after the stroke. The grip strength for each test group after 24- and 48-hours post-operation are shown in Table 17 and FIGS. 14A-14B. Compared with the sham test group, the model test group (vehicle-treated) showed a significantly lower grip strength at both 24- and 48-hours post-operation (p<0.01; Table 17). A significantly higher grip strength was observed for the ALMB-0166 25 mg/kg and 50 mg/kg tests group when compared to the model test group at 24 hours post-procedure (p<0.01; Table 17 and FIG. 2A). At 48 hours post-operation, a significantly higher grip strength was observed for the butylphthalide and the ALMB-0166 25 mg/kg and 50 mg/kg tests groups, as compared to the model test group (Table 17 and FIG. 14B).

TABLE 17
Grip strength of animal test groups
over study period (Mean ± SEM, n = 12).

| | Grip Strength (g) | | | |
|---|---|---|---|---|
| Group | 24 h | | 48 h | |
| Sham (no stroke) | 616.67 ± | 76.98 | 584.11 ± | 60.60 |
| Model (vehicle-treated) | 334.82 ± | 80.12^{##} | 362.44 ± | 56.84^{##} |
| Butylphthalide 10 mg/kg | 404.73 ± | 111.68 | 419.93 ± | 71.14* |
| ALMB-0166 15 mg/kg | 338.70 ± | 93.15 | 380.20 ± | 51.19 |
| ALMB-0166 25 mg/kg | 443.49 ± | 80.35** | 480.19 ± | 98.08** |
| ALMB-0166 50 mg/kg | 471.84 ± | 124.44** | 499.28 ± | 79.45** |

Note:
^{##}p < 0.01 vs. sham;
\hat{ } p < 0.63 vs. model;
*p < 0.05 vs. model; and
**p < 0.01 vs. model.

### Effect of Treatment with Anti-Cx43 Antibody on Rotarod Test Score of Rats after MCAO

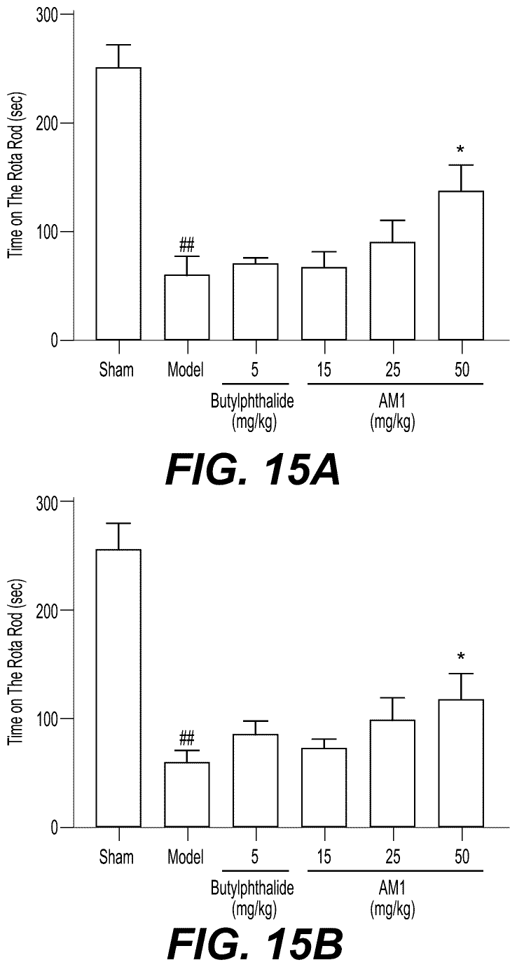

A rotarod test was performed as a third assessment of neurological function. The rotarod test scores for each test group after 24- and 48-hours post-operation are shown in Table 18 and FIGS. 15A-3B. A statistically significant difference was observed between the sham group and the model test group at both time points (p<0.01; Table 18). At both 24-hours and 48-hours post-operation, a significantly higher rotarod test score was observed for the ALMB-0166 50 mg/kg test group, as compared to the model test group (p<0.05; Table 18 and FIGS. 15A-15B).

TABLE 18
Rotarod test score of animal test groups
over study period (Mean ± SEM, n = 12).

| | Time (s) | | | |
|---|---|---|---|---|
| Group | 24 h | | 48 h | |
| Sham (no stroke) | 252.33 ± | 62.92 | 256.50 ± | 79.02 |
| Model (vehicle-treated) | 60.17 ± | 62.69^{##} | 60.25 ± | 39.72^{##} |
| Butylphthalide 10 mg/kg | 69.83 ± | 20.70 | 86.42 ± | 42.41 |
| ALMB-0166 15 mg/kg | 67.83 ± | 46.87 | 74.50 ± | 27.85 |
| ALMB-0166 25 mg/kg | 91.08 ± | 68.23 | 98.83 ± | 71.09 |
| ALMB-0166 50 mg/kg | 137.17 ± | 88.02* | 119.00 ± | 79.06* |

Note:
^{##}p < 0.01 vs. sham;
\hat{ } p 0.69 vs. model; and
*p < 0.05 vs. Model

### Effect of Treatment with an Anti-Cx43 Antibody on Cerebral Infarct Volume of Rats after MCAO

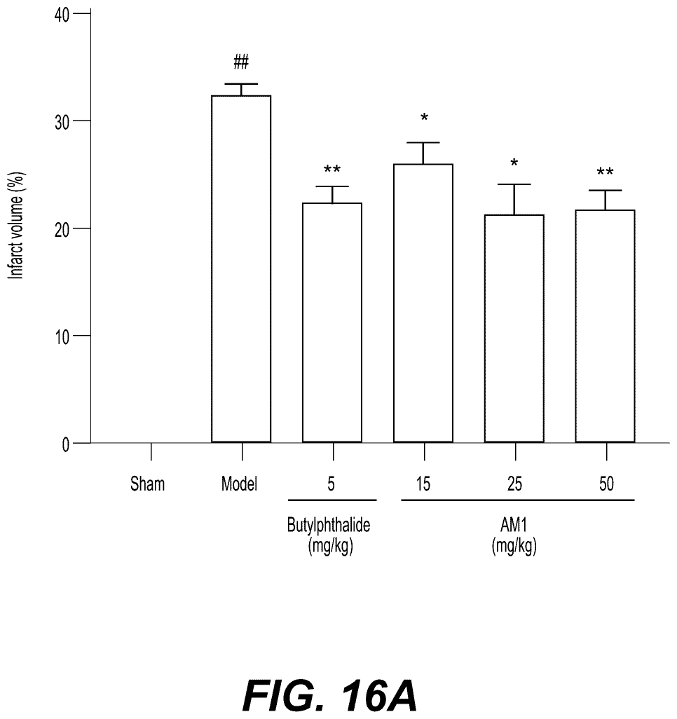

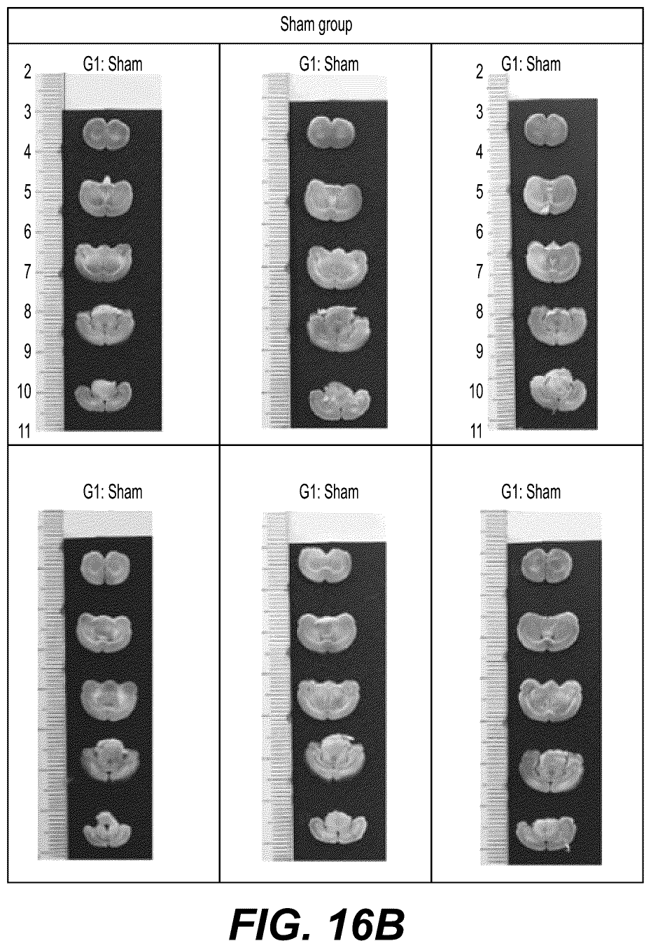

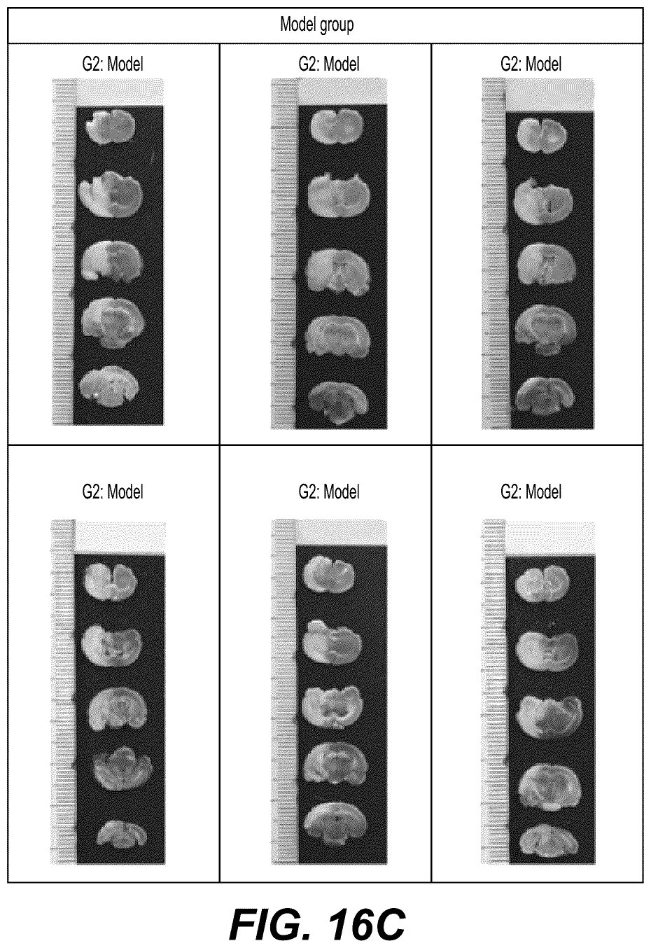

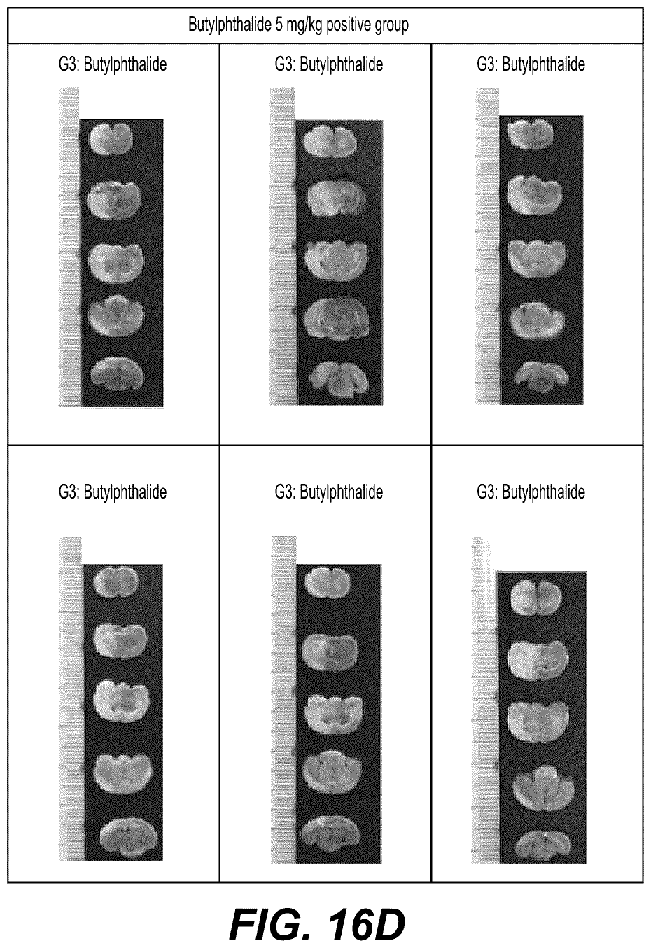

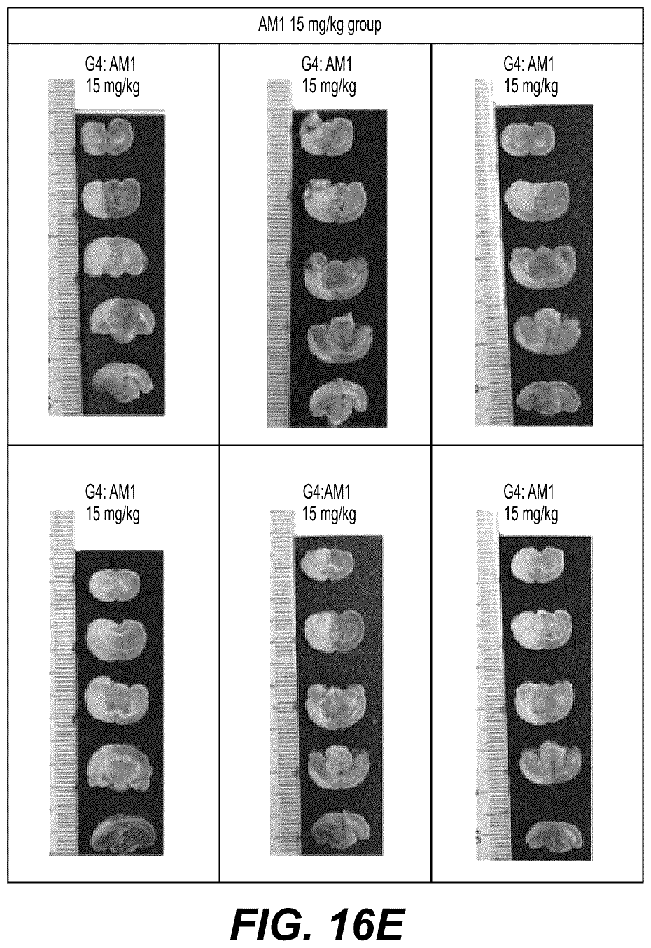

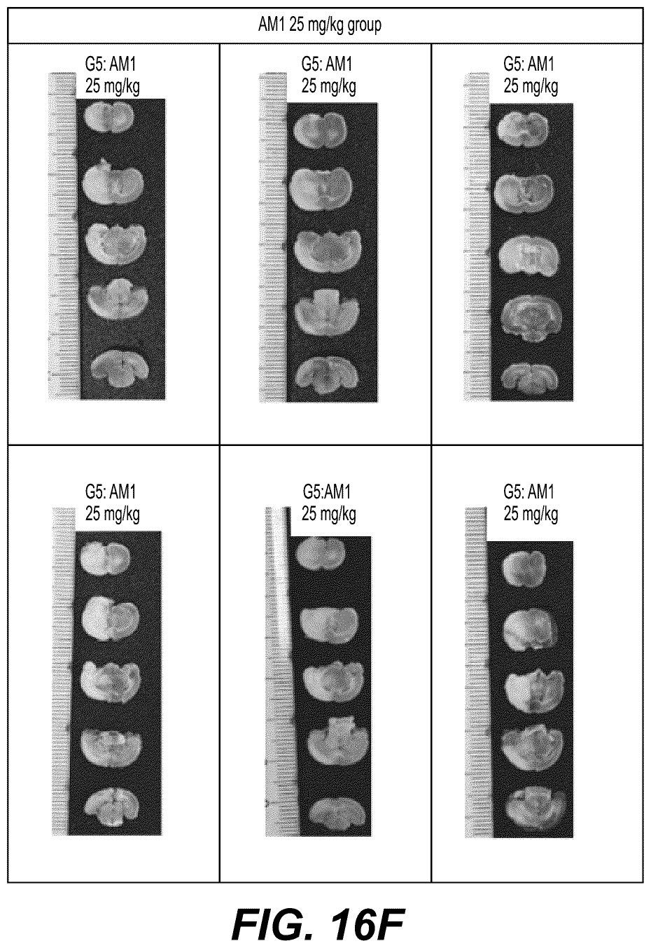

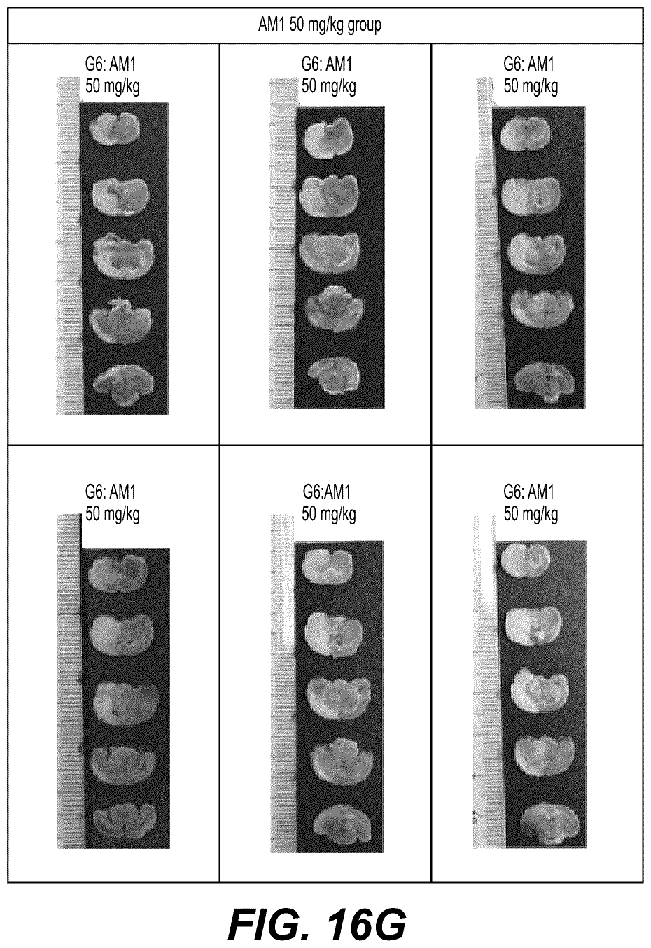

The cerebral infarct volume and the improvement on the rate of infarction of each test group was measured following 48-hours post-operation (Table 19 and FIGS. 16A-16G). A statistically significant difference in cerebral infarct volume was observed between the sham group and the model test group, with no cerebral infarct volume detected for the rats in the sham group (p<0.01; Table 19 and FIG. 16A). The cerebral infarct volume was significantly lower in the butylphthalide test group and all ALMB-0166 test groups, as compared to the model test group (Table 19 and FIG. 16A). An improvement in the rate of infarction was also observed for the butylphthalide test group as well as all ALMB-0166 test groups (Table 19). Table 19. Cerebral infarct volume of animal test groups (Mean±SEM, n=6).

TABLE 19
Cerebral infarct volume of
animal test groups (Mean ± SEM, n = 6).

| | Cerebral | | Improvement |
|---|---|---|---|
| | Infarction | | rate of |
| Group | Volume (%) | | infarction (%) |
| Sham (no stroke) | 0.00 ± | 0.00 | — |
| Model (vehicle-treated) | 32.41 ± | 2.50^{##} | — |
| Butylphthalide 10 mg/kg | 22.41 ± | 3.68** | 30.84 |
| ALMB-0166 15 mg/kg | 26.02 ± | 4.78* | 19.72 |
| ALMB-0166 25 mg/kg | 21.18 ± | 7.13* | 34.64 |
| ALMB-0166 50 mg/kg | 21.70 ± | 4.63** | 33.06 |

Note:
^{##}p < 0.01 vs. sham;
*p < 0.05 vs. model; and
**p < 0.01 vs. model.

### Effect of Treatment with an Anti-Cx43 Antibody on Brain Water Content of Rats after MCAO

The brain water content and the brain water ratio of each test group was measured following 48-hours post-operation (Table 20 and FIG. 17). A significant difference in brain water content and brain water ratio were observed between the sham group and the model test group (p<0.01; Table 20 and FIG. 17). Compared with the model test group, the butylphthalide test group and all ALMB-0166 test groups showed a lower brain water content and lower brain water ration (Table 20 and FIG. 17).

TABLE 20
Brain water content of animal
test groups (Mean ± SEM, n = 6).

| | Brain water | | Brain water | |
|---|---|---|---|---|
| Group | content (mg) | | ratio (%) | |
| Sham (no stroke) | 1084.17 ± | 43.98 | 77.63 ± | 0.97 |
| Model (vehicle-treated) | 1286.00 ± | 55.33^{##} | 80.06 ± | 1.16^{##} |
| Butylphthalide 10 mg/kg | 1282.00 ± | 75.80 | 78.80 ± | 2.37 |
| ALMB-0166 15 mg/kg | 1246.17 ± | 101.29 | 80.63 ± | 1.82 |
| ALMB-0166 25 mg/kg | 1230.83 ± | 100.57 | 79.24 ± | 1.15 |
| ALMB-0166 50 mg/kg | 1254.83 ± | 89.75 | 79.92 ± | 2.39 |

Note:
^{##}p < 0.01 vs. sham.

### Effect of Treatment with an Anti-Cx43 Antibody on Brain Volume of Rats after MCAO

The brain volume for each test group was measured following 48-hours post-operation (Table 21 and FIG. 18). A statistically significant difference in brain volume was observed between the sham group and the model test group (p<0.01; Table 21 and FIG. 18). The ALMB-0166 50 mg/kg test group showed a significantly lower than in rats from the model test group (p<0.05; Table 21 and FIG. 18).

TABLE 21
Brain volume of animal test groups (Mean ± SEM, n = 12).

| Group | Brain volume (mL) |
|---|---|
| Sham (no stroke) | 1.415 ± 0.05 |
| Model (vehicle-treated) | 1.623 ± 0.07^{##} |
| Butylphthalide 10 mg/kg | 1.597 ± 0.11 |
| ALMB-0166 15 mg/kg | 1.586 ± 0.10 |
| ALMB-0166 25 mg/kg | 1.572 ± 0.06 |
| ALMB-0166 50 mg/kg | 1.537 ± 0.09 |

Note:
^{##}p < 0.01 vs. sham; and
*p < 0.05 vs. model.

### Conclusions

Treatment of a rat model of stroke induced by MCAO with the anti-Cx43 antibody ALMB-0166 at dosages of 15 mg/kg, 25 mg/kg and 50 mg/kg led to improvements in neurological function post-stroke in a dose-dependent manner. These improvements in neurological function were superior to those observed for treatment of the rats with butylphthalide. Administration of ALMB-0166 also led to improvements in the cerebral infarct volume post-stroke in a dose-dependent manner. In addition, administration of ALMB-0166 led to improvements in the acute brain edema of the rats, as assessed by brain water content and brain volume.

### Example 4. Neuroprotective Effects of Treatment with an Anti-Cx43 Antibody on a Rat Model of Stroke

This Example describes the evaluation of the neuroprotective effects of an anti-Cx43 antibody in a rat model of stroke.

### Study Objective

The purpose of the study was to test the neuroprotective effects of a dosing regimen of the anti-Cx43 antibody ALMB-0166 on in a rat model of stroke induced by middle cerebral artery occlusion (MCAO).

### Regulatory Compliance

The experiment complied with Standard Operating Procedures (SOPs).

### Materials and Methods

### Test Materials

The anti-Cx43 antibody used in the study was ALMB-0166 (AlaMab Therapeutics Inc.). Butylphthalide and sodium chloride (CSPC NBP Pharmaceutical Group Co., Ltd.) was used as a positive control. Other materials used in the study included 0.9% normal saline (Anhui Shuanghe Pharmaceutical Co., Ltd), sterile water for injection (Sichuan Kelun Pharmaceutical Co., Ltd.), isoflurane (RWD Life Science Inc.), phosphate buffered saline (Thermo, batch no.), 2,3,5-triphenyltetrazolium chloride (TTC; Bide Pharmaceutical Technology, Co., Ltd.), and a 40% formaldehyde solution (Sinopharm Chemical Reagent Co., Ltd.)

For animal dosing, solutions of ALMB-0166 at 1 mg/mL, 2.5 mg/mL and 5 mg/mL were prepared by diluting the antibody stock solution with PBS. A 1% w/v TTC solution was also prepared for the study.

### Equipment

Equipment used in this study included an inhalation anesthesia apparatus (Midmark Corporation, type VWR), a mouse thread bolt (Guangzhou Jialing Biotechnology Co., Ltd., type L3400), and a rat brain mold (RWD Life Sciences Inc., type 175-300).

### Animals and Husbandry

Male Sprague Dawley (SD) rats (Specific Pathogen Free grade; Shanghai Slac Laboratory Animal Co. Ltd) were obtained in the study. Each rat was weight at the start of treatment, with the average weight ranging from 200-250 grams. Upon arrival at the test facility, the animals were housed at five rats per cage and acclimated for one to three days.

Animals were kept in an environment set to maintain a temperature of 23±2° C., with humidity of 40-70%, and a 12-hour light/12-hour dark cycle. The 12-hour dark cycle was temporarily interrupted to accommodate study procedures. SPF Rat Growth Breeding Feed (Beijing Keao Xieli Feed Co., Ltd)) was provided ad libitum throughout the in-life part of the study. Reverse Osmosis water was available ad libitum.

The animals used in the study were selected based on overall health and acclimation to caging. The rats were fasted for 12 hours before experimental modeling and were free to access both food and water afterwards.

A total of 36 rats were selected for participation in the study. Studies using common mammalian laboratory animals such as rats, dogs, and monkeys are essential and routinely used for the evaluation of the pharmacokinetics, pharmacodynamics and toxicology characteristics of new chemical entities. The number of animals in each test group was the minimal number needed for the assessment of variability between test animals.

### Study Design

### Animal Groups and Drugs

For the study, animal groups were formed as indicated in Table 22 below. The first administration of the respective treatments was started 30 minutes (Day 1 of study) after the MCAO procedure of the rats. For the butylphthalide group, subsequent doses were administered every day after for 14 days. For the ALMB-0166 test groups, a second dose of the antibody was administered at Day 4 post-stroke (Day 4 of the study).

TABLE 22
Treatment and dosing information for animal test groups.

| | | | | | | Dosing route, |
|---|---|---|---|---|---|---|
| | | Animal | Dose | Dose volume | Concentration | frequency, and |
| Group | Treatment | Number | (mg/kg) | (mL/kg) | (mg/mL) | period |
| 1 | Sham (no stroke) | 6 | — | — | — | — |
| 2 | Model (vehicle- | 6 | — | 10 | — | Intravenous |
| | treated) | | | | | (i.v.), at |
| | | | | | | days 1 and 4 |
| 3 | Butylphthalide 5 | 6 | 5 | 20 | 0.25 | Intravenous |
| | mg/kg | | | | | (i.v.), twice |
| | | | | | | daily (Bid) |
| 4 | ALMB-0166 15 | 6 | 15 | 10 | 1.5 | Intravenous |
| | mg/kg | | | | | (i.v.), at |
| | | | | | | days 1 and 4 |
| 5 | ALMB-0166 25 | 6 | 25 | 10 | 2.5 | Intravenous |
| | mg/kg | | | | | (i.v.), at |
| | | | | | | days 1 and 4 |
| 6 | ALMB-0166 50 | 6 | 50 | 10 | 5 | Intravenous |
| | mg/kg | | | | | (i.v.), at |
| | | | | | | days 1 and 4 |

### Models and Methods

Thirty-six (36) SD rats received behavioral training after adaptive feeding period and before the operation to establish stroke models. The behavioral training included beam-walking, grip and rotarod tests. The experimental rats were then randomly divided into 6 groups according to the body weight, with 6 rats in each test group as described in Table 15.

The rats were fasted overnight before modeling and were anesthetized with isoflurane. Right middle cerebral artery occlusion (MCAO) was induced in each rat using the intraluminal filament technique to mimic focal cerebral ischemic stroke. After 2 hrs, reperfusion was allowed by removing the occlusion.

Administration of butylphthalide was performed 0.5 hrs after the infarction, and every day after the operation (14 days). Administration of ALMB-0166 and the vehicle treatment was performed at 0.5 hrs and at Day 4 after the operation.

The body weight of rats was recorded before the operation (Day 0 of study) and on Days 2, 4, 7, and 11 after the procedure. The rats were scored by the Longa method at Day 1 (24-hours) post-procedure. The neurological function was evaluated at Days 1, 2, 4, 7, and 14 after the procedure, and was assessed by beam-walking, grip strength, and rotarod tests. At Day 14 after reperfusion, the whole brain tissue of all rats was harvested to assess the cerebral infarct area by TTC staining. The total infarct volume was determined, and the infarct volume ratio was calculated by dividing the total infarct volume by the whole brain volume. For calculating the infarct volume rate, the difference in infarct volume ratio between the model group and the corresponding administration group was divided by the infarct volume ratio of the model group.

### Statistical Analysis

The neurological function scores and the cerebral infarct volume were expressed as the mean and standard error (Mean±SEM). The homogeneity of variance was tested in the different test groups. If the variance was homogeneous, one-way analysis of variance was performed. If the variance was heterogeneous, T-test was used for subsequent analysis. p<0.05 was considered to have a significant difference, and p<0.01 was considered to have a very significant difference. The final data were plotted with GraphPad Prism 6 software.

### Results

General Clinical Symptoms of Rats after Administration

After the procedure, the Zea-Longa score of rats in each administration test group was about 2-3, while the Zea-Longa score for the sham group was 0. All animals had no visible abnormalities during the experimental period.

### Effect of an Anti-Cx43 Antibody Dosing Regimen on Body Weight of Rats after MCAO

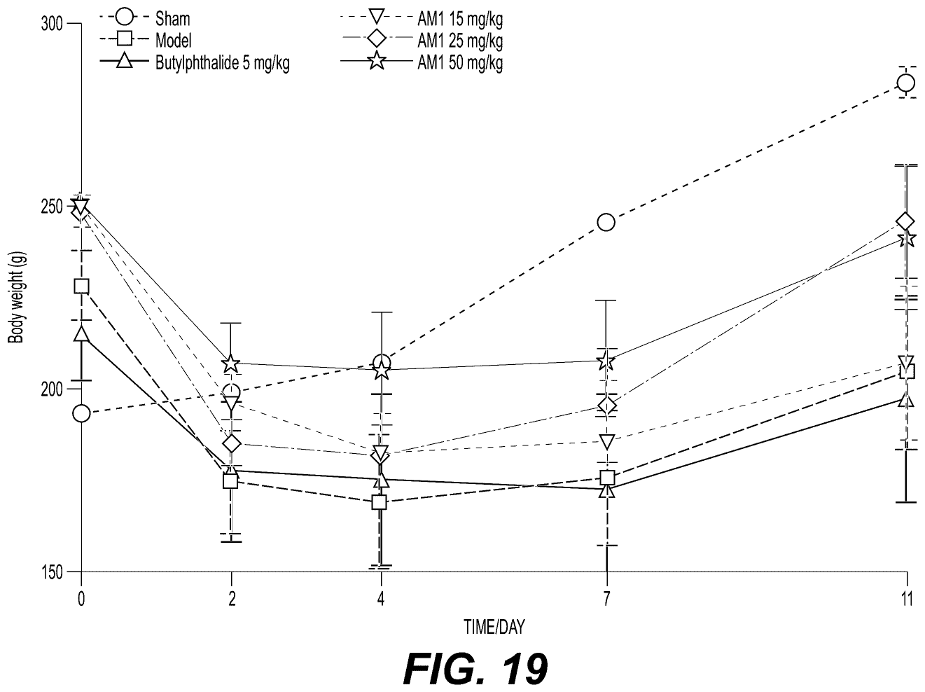

Body weight of rats in each test group was assessed at Days 0, 2, 4, 7 and 11 of the study (Table 23 and FIG. 19). The body weight of rats in the sham group increased continuously during the study period, while the body weight of rats in the model test group decreased during Days 2 and 4 post-procedure and increased at Days 7 and 11 post-procedure (Table 23 and FIG. 19). The body weight of animals in the butylphthalide and ALMB-0166 test groups decreased initially and increased following administration of a second dose of the respective treatment at Day 4 post-procedure (Table 23 and FIG. 19). No statistically significant difference was between the body weight of animals in the butylphthalide and ALMB-0166 test groups as compared to animals in the model test group.

TABLE 23
Weight of animal test groups over study period (Mean ± SEM, n = 6)

| | Weight (g) | | | | |
|---|---|---|---|---|---|
| Group | Day 0 | Day 2 | Day 4 | Day 7 | Day 11 |
| Sham (no | 193.33 ± 5.35 | 198.67 ± 3.88 | 207.33 ± 6.92 | 246.00 ± 5.90 | 284.17 ± 9.64 |
| stroke) | | | | | |
| Model | 228.33 ± 23.17 | 174.50 ± 34.75 | 169.33 ± 44.37 | 175.50 ± 44.77^{#} | 204.83 ± 51.94^{#} |
| (vehicle-treated) | | | | | |
| Butylphthalide | 215.73 ± 34.34 | 177.17 ± 46.84 | 175.33 ± 56.38 | 172.67 ± 64.22 | 197.17 ± 67.82 |
| 10 mg/kg | | | | | |
| ALMB-0166 | 249.17 ± 10.91 | 196.00 ± 19.22 | 182.50 ± 26.14 | 185.83 ± 39.97 | 207.33 ± 52.08 |
| 15 mg/kg | | | | | |
| ALMB-0166 | 248.17 ± 6.43 | 185.17 ± 15.41 | 181.83 ± 27.86 | 195.50 ± 37.64 | 246.33 ± 38.95 |
| 25 mg/kg | | | | | |
| ALMB-0166 | 252.22 ± 3.27 | 207.17 ± 27.30 | 205.67 ± 37.66 | 208.50 ± 39.22 | 242.00 ± 48.90 |
| 50 mg/kg | | | | | |

Note:
^{#}p < 0.05 vs. sham.

### Effect of an Anti-Cx43 Antibody Dosing Regimen on Beam-Walking of Rats after MCAO

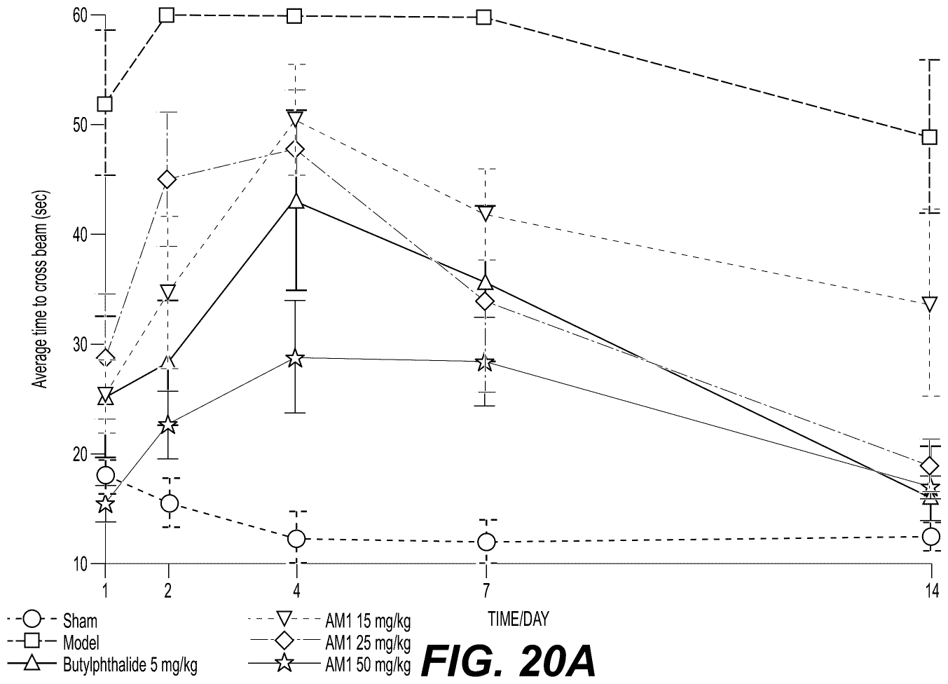

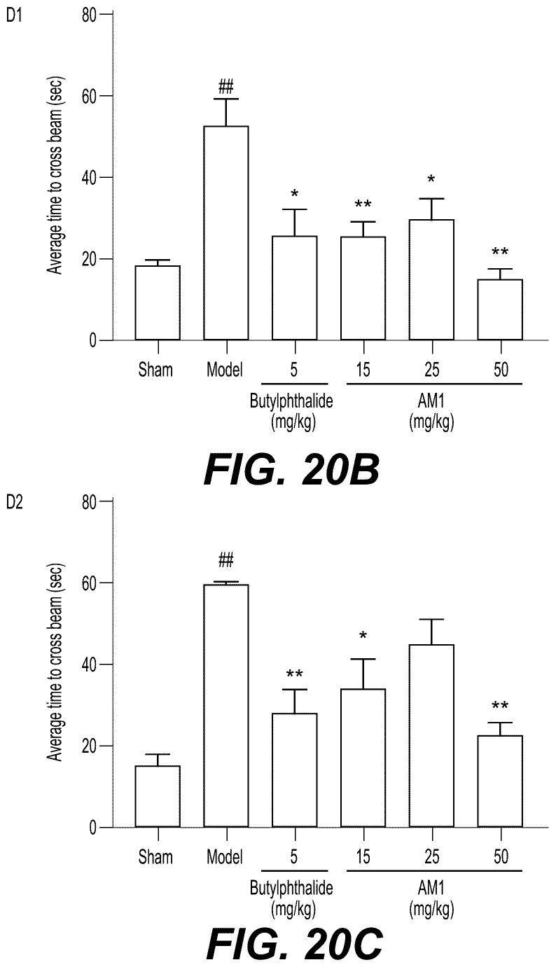

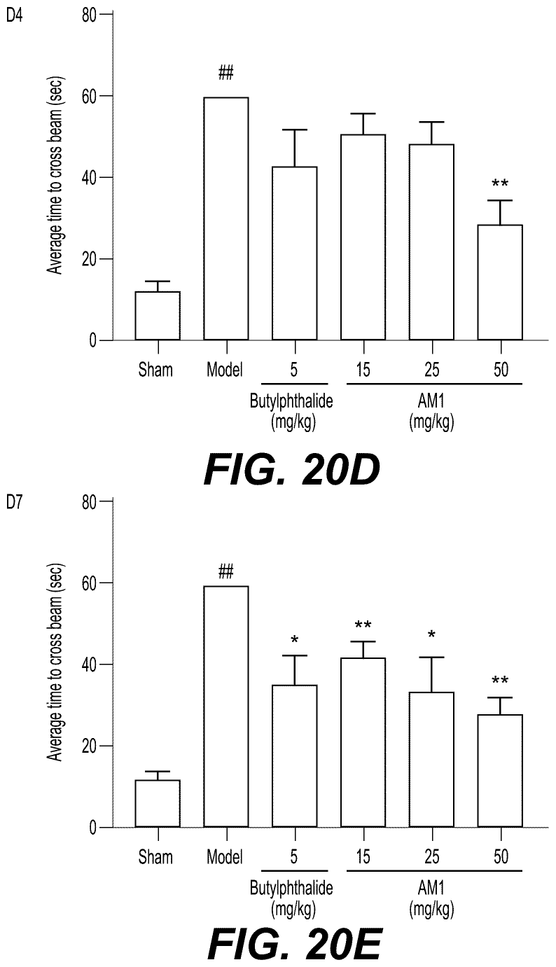

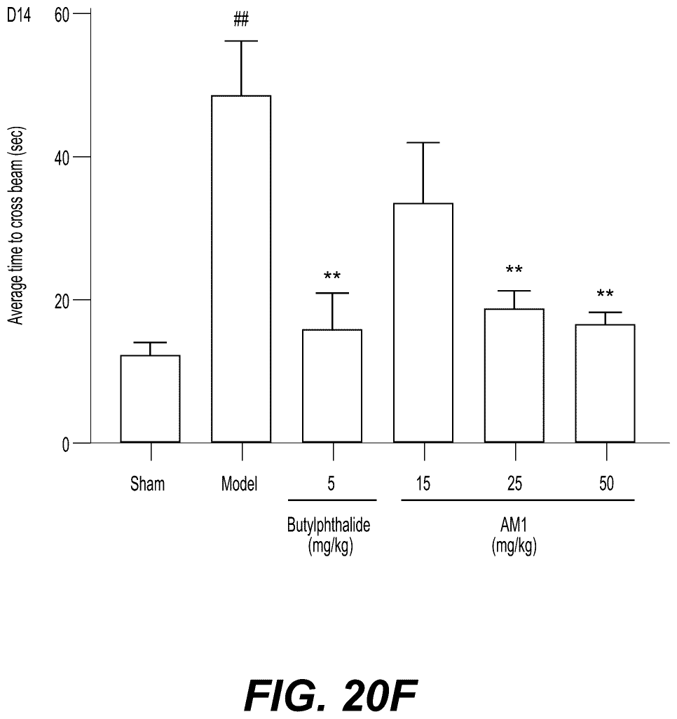

Beam-walking test was used to assess neurological function the rat stroke model after the stroke. The beam-walking time of each test group at Days 1, 2, 4, 7, and 14 post-operation are shown in Table 24 and FIGS. 20A-20F. A statistical difference was observed in the beam-walking time of the model (vehicle-treated) test group as compared to the sham group at all examined days post-operation (p<0.01; Table 24). The beam-walking time of rats in the butylphthalide test group was significantly lower than that of rats in the model test group at Days 1, 2, 4 and 14 post operation (p<0.05; Table 24 and FIGS. 20A-20F). When compared to the model test group, a lower beam-walking time was observed for the ALMB-0166 15 mg/kg, with a significant difference observed at Days 1, 2 and 7 post-operation (Table 24 and FIGS. 20A-20F). The ALMB-0166 25 mg/kg also showed a lower beam walking time than the model test group, with a significant difference observed on Days 1, 7 and 14 post-operation (Table 24 and FIGS. 20A-20F). When compared to the model test group, a significantly lower beam-walking time was observed for the ALMB-0166 50 mg/kg test group at all examined days (Table 24 and FIGS. 20A-20F).

TABLE 24
Beam-walking time of animal test groups over study period (Mean ± SEM, n = 6)

| | Time (s) | | | | | | | | | |
|---|---|---|---|---|---|---|---|---|---|---|
| Group | Day 0 | | Day 2 | | Day 4 | | Day 7 | | Day 14 | |
| Sham (no | 18.01 ± | 4.26 | 15.52 ± | 5.25 | 12.38 ± | 5.82 | 12.04 ± | 4.95 | 12.57 ± | 3.31 |
| stroke) | | | | | | | | | | |
| Model | 51.11 ± | 16.51^{##} | 60.00 ± | 0.00^{##} | 60.00 ± | 0.00^{##} | 60.00 ± | 0.00^{##} | 48.92 ± | 17.22^{##} |
| (vehicle- | | | | | | | | | | |
| treated) | | | | | | | | | | |
| Butylphthalide | 23.13 ± | 18.17* | 28.31 ± | 14.02** | 43.23 ± | 20.35 | 35.65 ± | 17.18* | 16.01 ± | 11.72** |
| 10 mg/kg | | | | | | | | | | |
| ALMB-0166 | 25.20 ± | 8.17** | 34.79 ± | 16.85* | 50.48 ± | 12.35 | 41.96 ± | 10.08** | 33.72 ± | 20.81 |
| 15 mg/kg | | | | | | | | | | |
| ALMB-0166 | 28.92 ± | 14.02* | 45.12 ± | 14.72 | 47.86 ± | 13.42 | 34.02 ± | 20.31* | 18.90 ± | 6.15** |
| 25 mg/kg | | | | | | | | | | |
| ALMB-0166 | 15.58 ± | 3.94** | 22.67 ± | 7.48** | 28.84 ± | 12.54** | 28.40 ± | 9.94** | 16.95 ± | 2.89** |
| 50 mg/kg | | | | | | | | | | |

Note:
^{##}p < 0.01 vs. sham;
*p < 0.05 vs. model; and
**p < 0.01 vs. model.

### Effect of an Anti-Cx43 Antibody Dosing Regimen on Grip Strength of Rats after MCAO

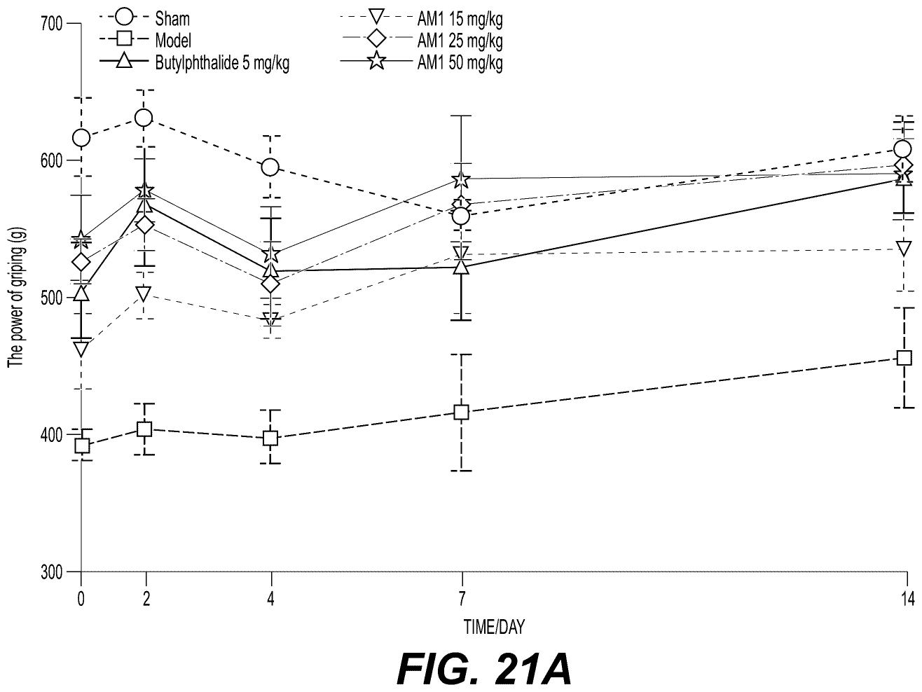

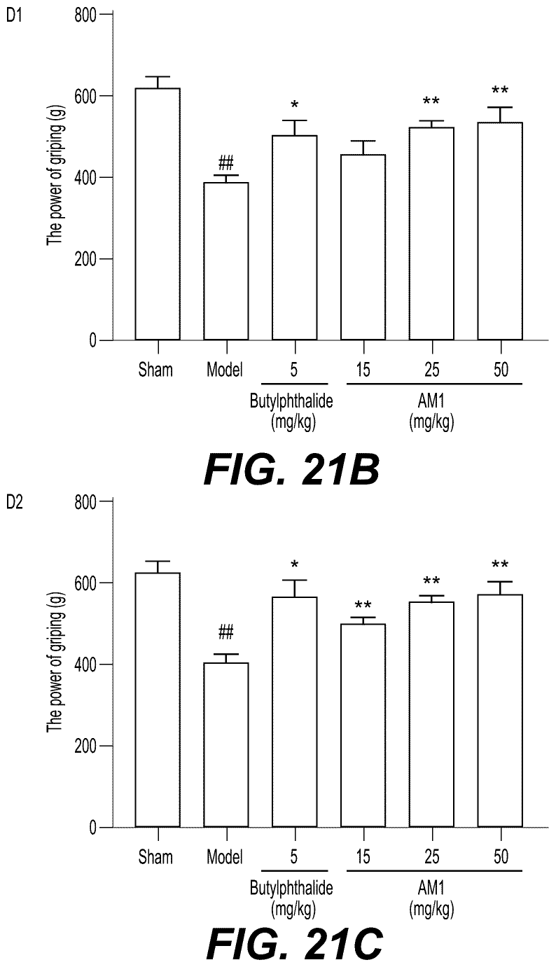

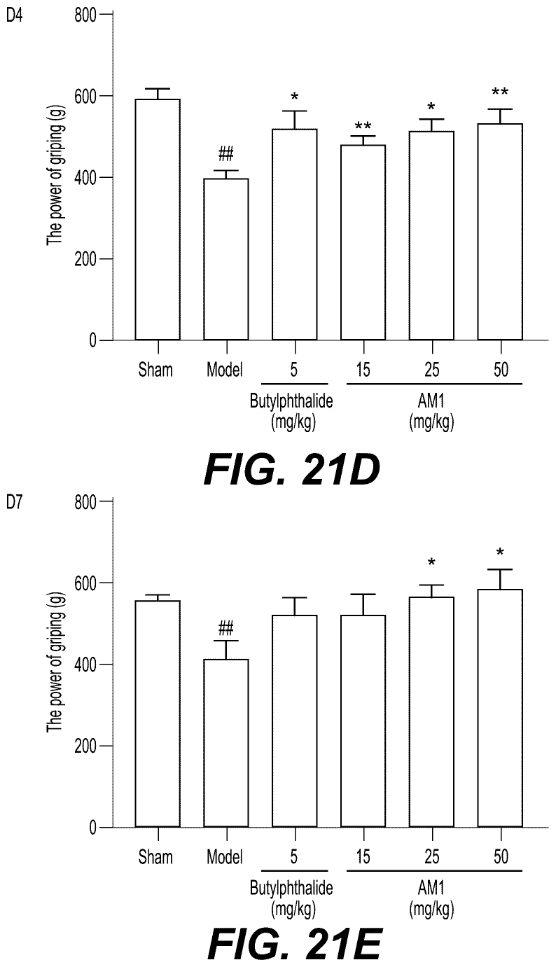

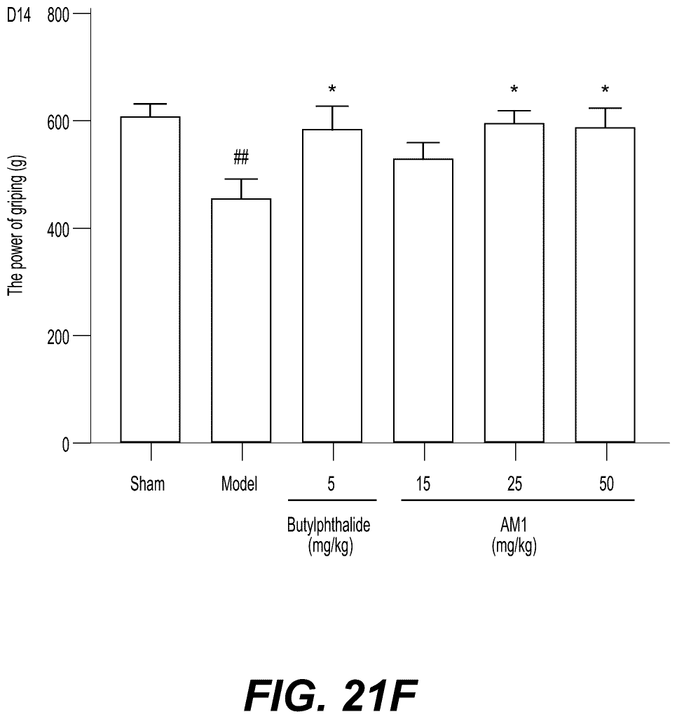

Grip strength measurements were taken as a second assessment neurological function the rat stroke model after the stroke. The grip strength for each test group at Days 1, 2, 4, 7 and 14 post-operation are shown in Table 25 and FIGS. 21A-21F. Compared with the sham test group, the model test group (vehicle-treated) showed a significantly lower grip strength at all examined days post-operation (Table 25). The grip strength of rats in the butylphthalide test group was significantly higher than that of rats in the model test group at Days 1, 2, 4 and 14 post operation (p<0.05; Table 25 and FIGS. 21A-21F). When compared to the model test group, a significantly higher grip strength was observed for the ALMB-0166 25 mg/kg and 50 mg/kg test groups at all examined days (Table 25 and FIGS. 21A-21F). The ALMB-0166 15 mg/kg showed a higher grip strength than the model test group at all examined days, with a significantly higher grip strength observed on Days 2 and 4 post-operation (Table 24 and FIGS. 21A-21F).

TABLE 25
Grip strength of animal test groups
over study period (Mean ± SEM, n = 6).

| | Grip Strength (g) | | | | | | | | | |
|---|---|---|---|---|---|---|---|---|---|---|
| Group | Day 0 | | Day 2 | | Day 4 | | Day 7 | | Day 14 | |
| Sham (no | 615.36 ± | 69.82 | 629.82 ± | 50.43 | 594.30 ± | 55.30 | 558.91 ± | 24.99 | 607.73 ± | 58.51 |
| stroke) | | | | | | | | | | |
| Model | 391.67 ± | 26.27^{##} | 402.77 ± | 44.78^{##} | 397.19 ± | 47.65^{##} | 415.22 ± | 103.31^{#} | 456.17 ± | 89.57#^{#} |
| (vehicle- | | | | | | | | | | |
| treated) | | | | | | | | | | |
| Butylphthalide | 504.65 ± | 83.03* | 565.99 ± | 106.98* | 520.36 ± | 89.50* | 523.26 ± | 97.69 | 585.23 ± | 102.52* |
| 10 mg/kg | | | | | | | | | | |
| ALMB-0166 | 459.42 ± | 67.78 | 500.68 ± | 42.02** | 482.46 ± | 30.46** | 529.71 ± | 102.53 | 533.51 ± | 69.76 |
| 15 mg/kg | | | | | | | | | | |
| ALMB-0166 | 525.43 ± | 38.23** | 555.19 ± | 47.05** | 509.56 ± | 74.81* | 567.18 ± | 73.61* | 596.42 ± | 44.61* |
| 25 mg/kg | | | | | | | | | | |
| ALMB-0166 | 542.68 ± | 75.62** | 577.07 ± | 57.73** | 531.93 ± | 81.23** | 585.90 ± | 113.41* | 589.68 ± | 81.16* |
| 50 mg/kg | | | | | | | | | | |

Note:
^{##}p < 0.01 vs. sham;
^{#}p < 0.05 vs. sham;
*p < 0.05 vs. model; and
**p < 0.01 vs. model.

### Effect of Treatment with Anti-Cx43 Antibody on Rotarod Test Score of Rats after MCAO

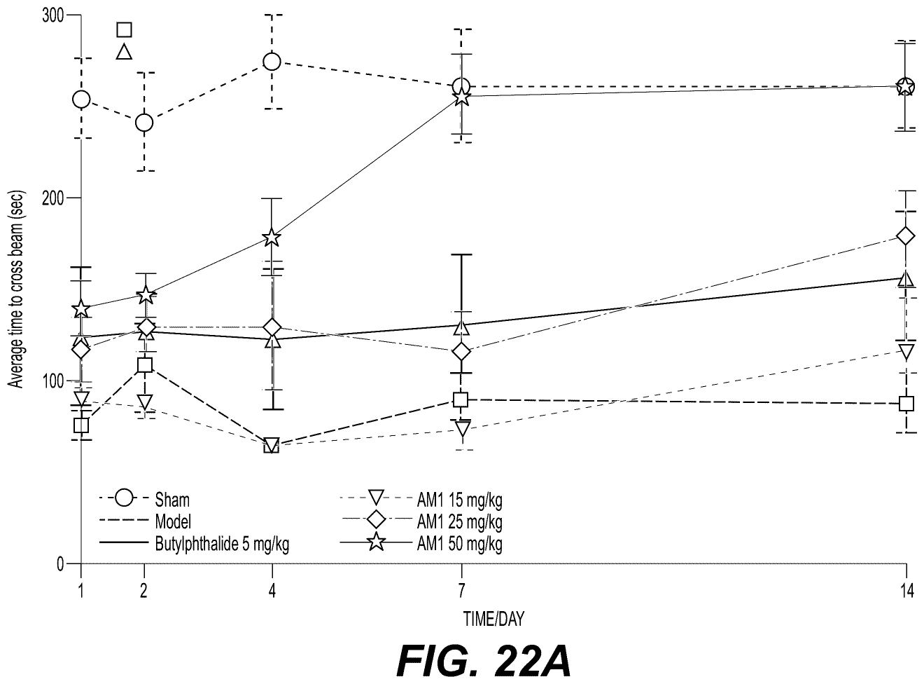

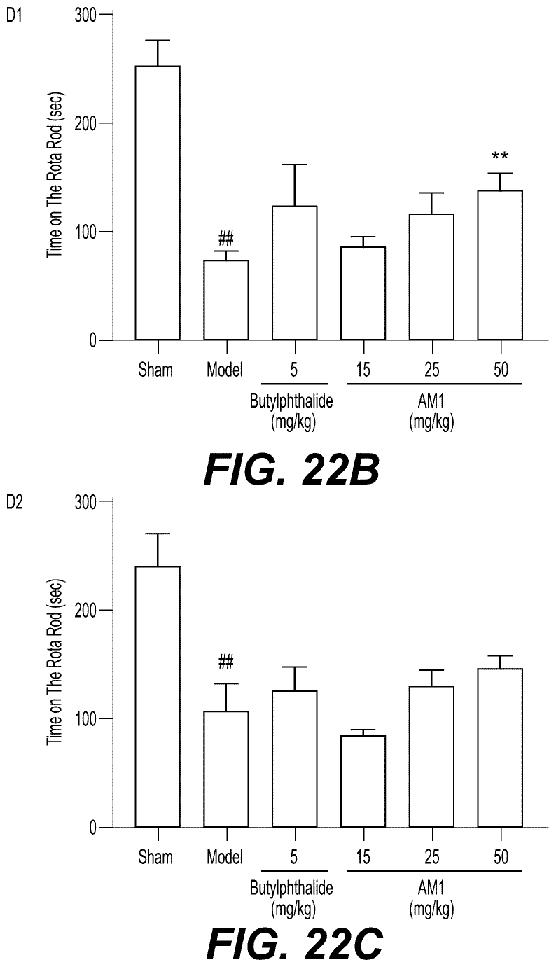

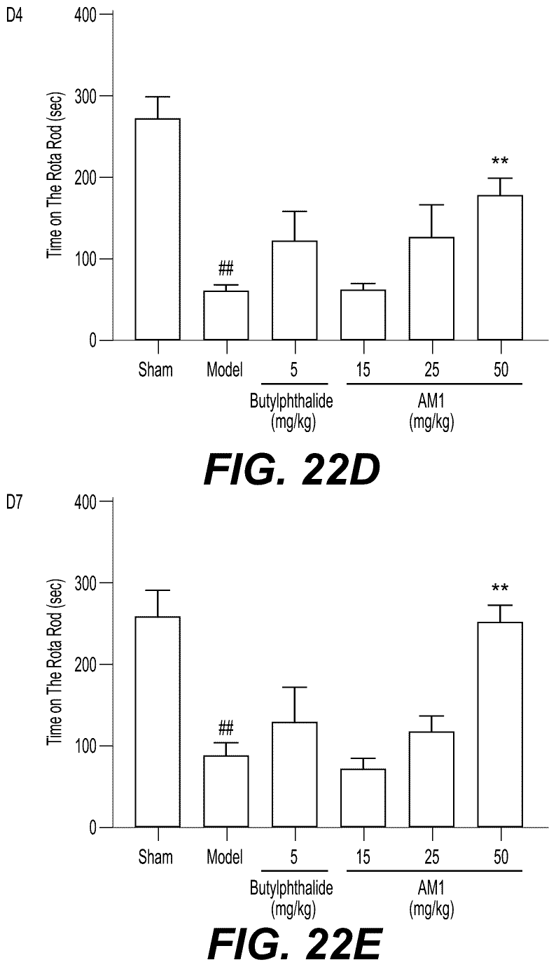

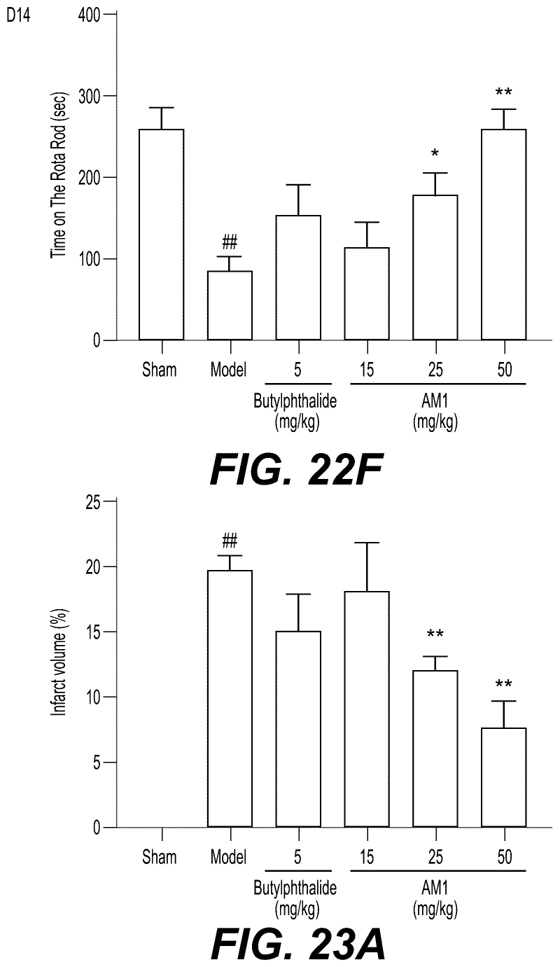

A rotarod test was performed as a third assessment of neurological function. The rotarod test scores for each test group at Days 1, 2, 4, 7 and 14 post-operation are shown in Table 26 and FIGS. 22A-22F. A statistically significant difference was observed between the sham group and the model test group at all examined days (p<0.01; Table 22). No statistically significant difference was observed between the model test group and the butylphthalide test group at all examined days (Table 26 and FIGS. 21A-22F). The ALMB-0166 25 mg/kg test group exhibited a higher rotarod test score than the model test group, with a significant difference observed at Day 14 post-operation (Table 26 and FIGS. 21A-22F). The ALMB-0166 50 mg/kg test group also exhibited a higher rotarod test score than the model test group, with a significant difference observed at Days 1, 4, 7 and 14 post-operation (Table 26 and FIGS. 21A-22F).

TABLE 26
Rotarod test score of animal test groups over study period (Mean ± SEM, n = 6).

| | Time (s) | | | | | | | | | |
|---|---|---|---|---|---|---|---|---|---|---|
| Group | Day 0 | | Day 2 | | Day 4 | | Day 7 | | Day 14 | |
| Sham (no | 274.7 ± | 53.1 | 241.7 ± | 65.8 | 274.7 ± | 62.1 | 261.8 ± | 75.6 | 262.0 ± | 59.0 |
| stroke) | | | | | | | | | | |
| Model | 75.0 ± | 19.0^{#} | 107.2 ± | 60.3^{##} | 64.0 ± | 10.7^{##} | 88.3 ± | 37.7^{##} | 87.3 ± | 38.7^{##} |
| (vehicle- | | | | | | | | | | |
| treated) | | | | | | | | | | |
| Butylphthalide | 124.5 ± | 93.5 | 126.8 ± | 52.4 | 122.2 ± | 95.3 | 130.7 ± | 96.7 | 157.3 ± | 86.9 |
| 10 mg/kg | | | | | | | | | | |
| ALMB-0166 | 87.3 ± | 18.4 | 84.4 ± | 14.9 | 64.2 ± | 13.7 | 73.3 ± | 28.6 | 117.3 ± | 68.8 |
| 15 mg/kg | | | | | | | | | | |
| ALMB-0166 | 117.2 ± | 44.4 | 130.7 ± | 35.6 | 130.0 ± | 87.0 | 116.2 ± | 54.1 | 179.0 ± | 63.4* |
| 25 mg/kg | | | | | | | | | | |
| ALMB-0166 | 140.0 ± | 38.2** | 147.3 ± | 28.0 | 179.3 ± | 51.7** | 257.0 ± | 53.7** | 262.3 ± | 56.2** |
| 50 mg/kg | | | | | | | | | | |

Note:
^{##}p < 0.01 vs. sham;
\hat{ } p 0.69 vs. model; and
*p < 0.05 vs. Model

### Effect of an Anti-Cx43 Antibody Dosing Regimen on Cerebral Infarct Volume of Rats after MCAO

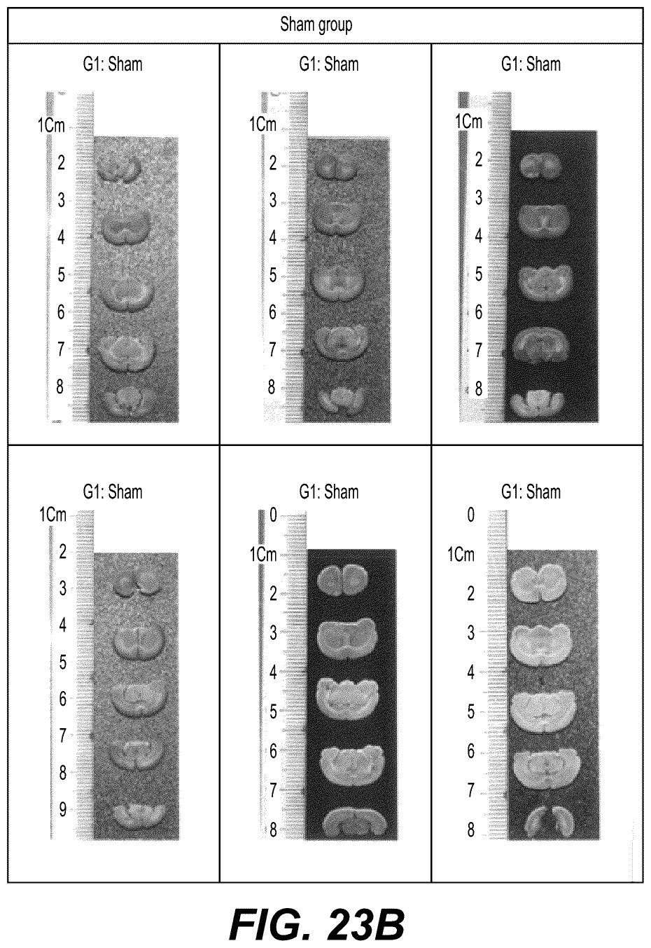

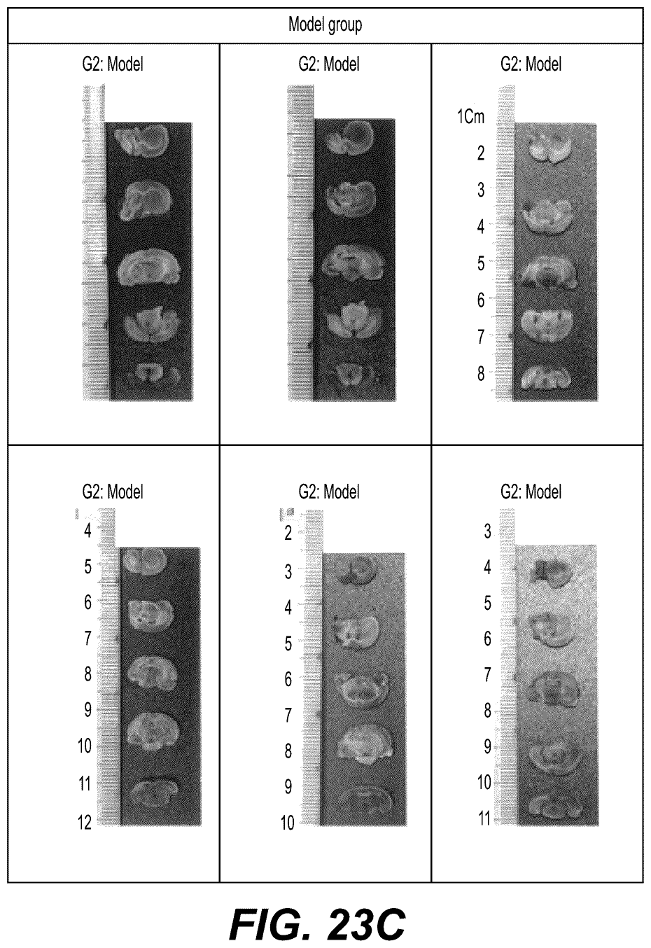

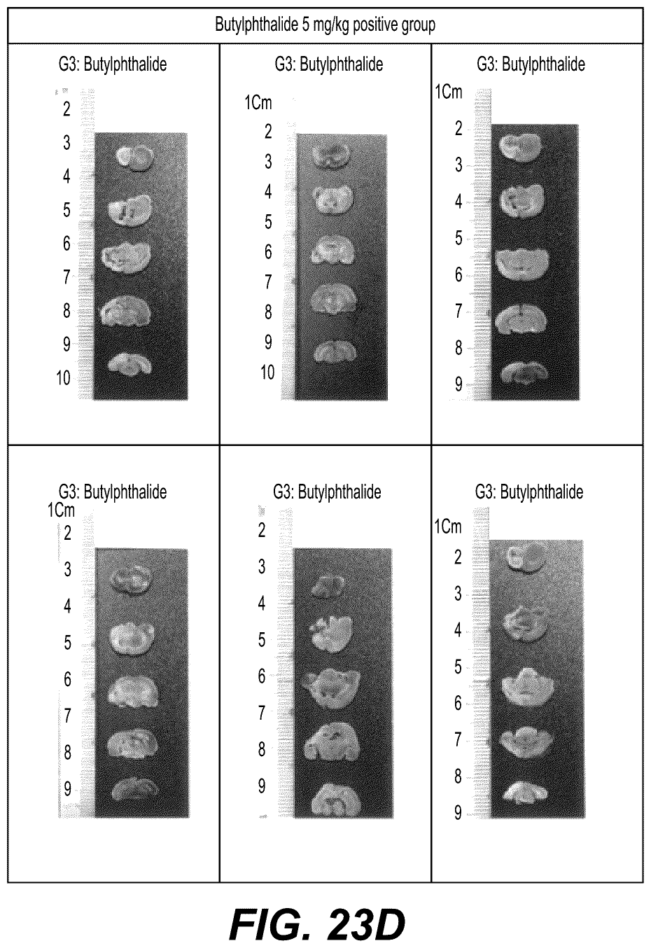

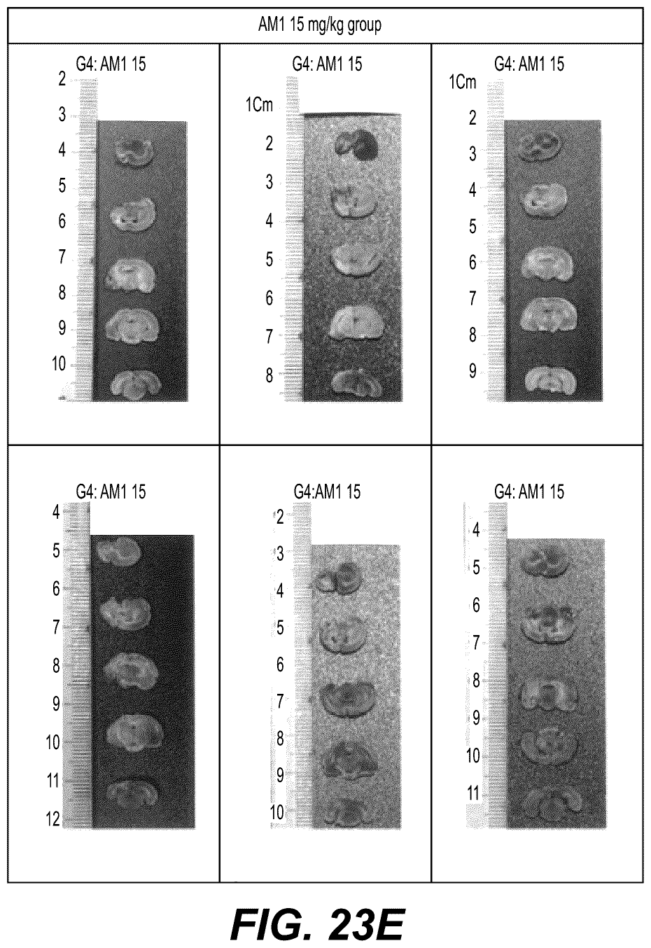

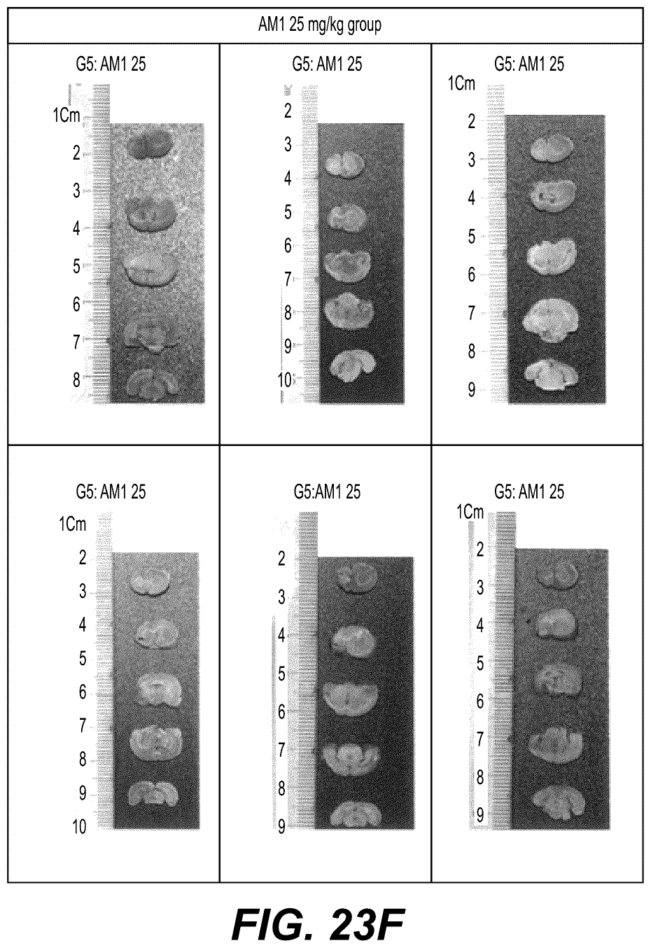

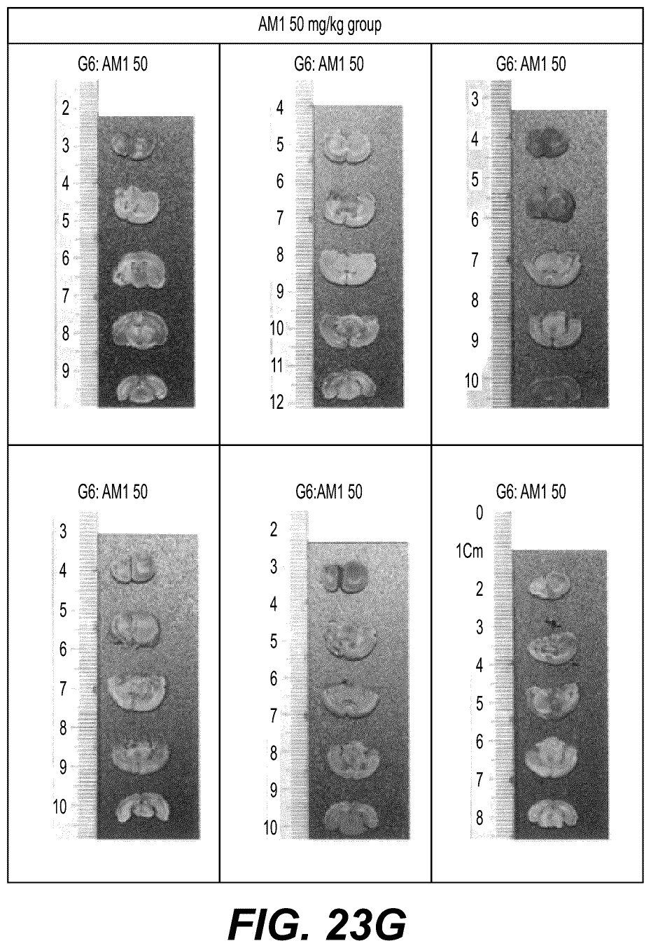

The cerebral infarct volume and the improvement on the rate of infarction of each test group was measured following 14-days post-operation (Table 27 and FIGS. 23A-23G). A statistically significant difference in cerebral infarct volume was observed between the sham group and the model test group, with no cerebral infarct volume detected for the rats in the sham group (p<0.01; Table 27 and FIG. 23A). The cerebral infarct volume was significantly lower in the ALMB-0166 25 mg/kg and 50 mg/kg test groups, as compared to the model test group (p<0.01; Table 27 and FIG. 23A). An improvement in the rate of infarction was also observed for the butylphthalide test group as well as all ALMB-0166 test groups (Table 27).

TABLE 27
Cerebral infarct volume of
animal test groups (Mean ± SEM, n = 6)

| | Cerebral | | Improvement |
|---|---|---|---|
| | Infarction | | rate of |
| Group | Volume (%) | | infarction (%) |
| Sham (no stroke) | 0.00 ± | 0.00 | — |
| Model (vehicle-treated) | 19.66 ± | 2.49^{##} | — |
| Butylphthalide 10 mg/kg | 15.01 ± | 6.50 | 23.64 |
| ALMB-0166 15 mg/kg | 18.04 ± | 8.90 | 8.24 |
| ALMB-0166 25 mg/kg | 12.09 ± | 2.62** | 38.50 |
| ALMB-0166 50 mg/kg | 7.64 ± | 5.05** | 61.11 |

Note:
^{##}p < 0.01 vs. sham; and
**p < 0.01 vs. model.

### Conclusions

Administration of anti-Cx43 antibody ALMB-0166 to a rat model of stroke induced by MCAO 30-minutes and 4-days post-infarction led to significant improvements in neurological function post-stroke in a dose-dependent manner. Administration of ALMB-0166 following this dosing regimen also led to improvements in the cerebral infarct volume post-stroke in a dose-dependent manner. The improvements in cerebral infarct volume post-stroke observed with ALMB-0166 at doses of 25 mg/kg and 50 mg/kg were superior to those observed with butylphthalide.

TABLE 28
Table of Sequences.

| SEQ ID | | |
|---|---|---|
| NO | Description | Amino Acid Sequence |
| 1 | HCDR1 | GYTFTSYY |
| 2 | HCDR2 | INPSNAGT |
| 3 | HCDR3 | TREGNPYYTMNY |
| 4 | LCDR1 | QSLLNSGNQKTY |
| 5 | LCDR2 | GAS |
| 6 | LCDR3 | QNDHSYPFT |
| 7 | VH sequence | EVQLVQSGAEVKKPGASVKVSCKASGYTFTSYYMYWVRQAPGQGLEWIGGINPSNAGT |
| | | NFNEKFKNRATLTVDKSTSTAYMELSSLRSEDTAVYYCTREGNPYYTMNYWGQGTLVT |
| | | VSS |
| 8 | VL sequence | DIVMTQSPDSLAVSLGERATISCKSSQSLLNSGNQKTYLAWYQQKPGQPPKLLIYGASTRE |
| | | SGVPDRFSGSGSGTDFTLTISSLQAEDVAVYYCQNDHSYPFTFGQGTKLEIK |
| 9 | Heavy chain of | EVQLVQSGAEVKKPGASVKVSCKASGYTFTSYYMYWVRQAPGQGLEWIGGINPSNaGTN |
| | Ab#K | FNEKFKNRATLTVDKSTSTAYMELSSLRSEDTAVYYCTREGNPYYTMNYWGQGTLVTVS |
| | | SASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPEAAGGP |
| | | SVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 10 | Light chain | DIVMTQSPDSLAVSLGERATISCKSSQSLLNSGNQKTYLAWYQQKPGQPPKLLIYGASTRE |
| | | SGVPDRFSGSGSGTDFTLTISSLQAEDVAVYYCQNDHSYPFTFGQGTKLEIKRTVAAPSVFI |
| | | FPPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 11 | Heavy chain of | EVQLVQSGAEVKKPGASVKVSCKASGYTFTSYYMYWVRQAPGQGLEWIGGINPSNgGTN |
| | Ab#E | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEFLGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 12 | Heavy chain of | EVQLVQSGAEVKKPGASVKVSCKASGYTFTSYYMYWVRQAPGQGLEWIGGINPSNaGTN |
| | Ab#G | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEFLGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 13 | Heavy chain of | EVQLVQSGAEVKKPGASVKVSCKASGYTFTSYYMYWVRQAPGQGLEWIGGINPSNgGTN |
| | Ab#I | FNEKFKNRATLTVDKSTSTAYMELSSLRSEDTAVYYCTREGNPYYTMNYWGQGTLVTVS |
| | | SASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPEAAGGP |
| | | SVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 14 | Heavy chain of | EVQLVQSGAEVKKPGASVKVSCKASGYTFTSYYMYWVRQAPGQGLEWIGGINPSNgGTN |
| | Ab#C | FNEKFKNRATLTVDKSTSTAYMELSSLRSEDTAVYYCTREGNPYYTMNYWGQGTLVTVS |
| | | SASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 15 | Heavy chain of | EVQLVQSGAEVKKPGASVKVSCKASGYTFTSYYMYWVRQAPGQGLEWIGGINPSNgGTN |
| | Ab#M | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEFEGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 16 | Heavy chain of | EVQLVQSGAEVKKPGASVKVSCKASGYTFTSYYMYWVRQAPGQGLEWIGGINPSNaGTN |
| | Ab#O | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEFEGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 17 | Heavy chain of | EVQLVQSGAEVKKPGASVKVSCKASGYTFTSYYMYWVRQAPGQGLEWIGGINPSNgGTN |
| | Ab#Q | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 18 | Heavy chain of | EVQLVQSGAEVKKPGASVKVSCKASGYTFTSYYMYWVRQAPGQGLEWIGGINPSNaGTN |
| | Ab#S | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 19 | Epitope | FLSRPTEKTI |

1. A method for treating a stroke in a subject, comprising administering to the subject at least one dose of an anti-connexin 43 (Cx43) antibody, wherein the anti-Cx43 antibody comprises heavy chain CDR sequences and light chain CDR sequences as follows:
HCDR1: SEQ ID NO: 1;
HCDR2: SEQ ID NO: 2;
HCDR3: SEQ ID NO: 3;
LCDR1: SEQ ID NO: 4;
LCDR2: SEQ ID NO: 5; and
LCDR3: SEQ ID NO: 6.

2. The method of claim 1, wherein the stroke is an ischemic stroke; or a hemorrhagic stroke.

3. (canceled)

4. The method of claim 1, wherein the at least one dose of the anti-Cx43 antibody is about 0.01 mg/kg to about 100 mg/kg.

5. The method of claim 1, wherein the at least one dose of the anti-Cx43 antibody is (i) about 15 mg/kg; (ii) about 25 mg/kg; or (iii) about 50 mg/kg.

6.-7. (canceled)

8. The method of claim 1, wherein the method comprises administering a first dose of the anti-Cx43 antibody (i) within 2 weeks from the stroke; (ii) within one week from the stroke; (iii) within 6 hours from the stroke; (iv) within 2 hours from the stroke; or (v) within 30 minutes from the stroke.

9.-12. (canceled)

13. The method of claim 1, wherein the method comprises (i) administering a second dose of the anti-Cx43 antibody at one day to four weeks after the first dose is administered; or (ii) administering a second dose of the anti-Cx43 antibody at least four days after the first dose is administered.

14. (canceled)

15. The method of claim 13, wherein the method comprises administering one or more subsequent doses of the anti-Cx43 antibody after the second dose is administered.

16. The method of claim 1, wherein the anti-Cx43 antibody is administered intravenously.

17. The method of claim 1, wherein (i) at least post-stroke criterion is assessed within three to six months after the at least one dose of the anti-Cx43 antibody is administered; and/or (ii) at least post-stroke criterion is assessed at least one day to two weeks after the at least one dose of the anti-Cx43 antibody is administered.

18. (canceled)

19. The method of claim 17, wherein at least post-stroke criterion is assessed at least one to two days after the at least one dose of the anti-Cx43 antibody is administered.

20. The method of claim 17, wherein the at least one post-stroke criterion comprises one or more of:
(a) neurological function,
(b) cerebral infarct volume, and
(c) cerebral edema.

21. The method of claim 17, wherein the at least one post-stroke criterion is improved after the at least one dose of the anti-Cx43 antibody is administered.

22. The method of claim 1, wherein the anti-Cx43 antibody comprises heavy chain variable sequence of SEQ ID NO: 7, and/or light chain variable sequence of SEQ ID NO: 8.

23. The method of one of claims 1-22 claim 1, wherein the anti-Cx43 antibody comprises heavy chain sequence of any one of SEQ ID NOs: 9, 12, 16 or 18, and/or light chain sequence of SEQ ID NO: 10.

24. The method of claim 1, wherein the anti-Cx43 antibody comprises heavy chain sequence of SEQ ID NO: 9, and/or light chain sequence of SEQ ID NO: 10.

25. A method for treating a stroke in a subject, comprising administering to the subject an effective amount of an anti-connexin 43 (Cx43) antibody, wherein the anti-Cx43 antibody is administered according to the following dosing regimen:
i) a first dose within one week from the stroke, and
ii) a second dose within a month from the stroke.

26. The method of claim 25, wherein the method comprises administering a first dose of the anti-Cx43 antibody (i) within 6 hours from the stroke; (ii) within 2 hours from the stroke; or (iii) within 30 minutes from the stroke.

27.-28. (canceled)

29. The method of claim 25, wherein the method comprises administering a second dose of the anti-Cx43 antibody at least four days after the first dose is administered.

30. The method of any one of claim 29, wherein the method further comprises administering one or more subsequent doses of the anti-Cx43 antibody after the second dose is administered.

31. The method of claim 25, wherein the first, second and/or subsequent dose of the anti-Cx43 antibody is about 0.01 mg/kg to about 100 mg/kg.

32. The method of claim 25, wherein the first, second and/or subsequent dose of the anti-Cx43 antibody is (i) about 15 mg/kg; (ii) about 25 mg/kg; or about 50 mg/kg.

33-34. (canceled)

35. The method of claim 25, wherein the anti-Cx43 antibody is administered intravenously.

36. The method of claim 25, wherein the stroke is (i) an ischemic stroke; or (ii) a hemorrhagic stroke.

37. (canceled)

38. The method of claim 25, wherein (i) at least post-stroke criterion is assessed within three to six months after the first, second and/or one or more subsequent doses of the anti-Cx43 antibody is administered; or (ii) wherein at least post-stroke criterion is assessed at least one day to two weeks after first, second and/or one or more subsequent doses of the anti-Cx43 antibody is administered.

39. (canceled)

40. The method of claim 38, wherein the at least one post-stroke criterion comprises one or more of:
(a) neurological function,
(b) cerebral infarct volume, and
(c) cerebral edema.

41. The method of claim 38, wherein at least one post-stroke criterion is improved after the first, second and/or one or more subsequent doses of the anti-Cx43 antibody is administered.

42. The method of claim 25, wherein the anti-Cx43 antibody comprises heavy chain CDR sequences and light chain CDR sequences as follows:
HCDR1: SEQ ID NO: 1;
HCDR2: SEQ ID NO: 2;
HCDR3: SEQ ID NO: 3;
LCDR1: SEQ ID NO: 4;
LCDR2: SEQ ID NO: 5; and
LCDR3: SEQ ID NO: 6.

43. The method of claim 25, wherein the anti-Cx43 antibody comprises heavy chain variable sequence of SEQ ID NO: 7, and/or light chain variable sequence of SEQ ID NO: 8.

44. The method of claim 25, wherein the anti-Cx43 antibody comprises heavy chain sequence of any one of SEQ ID NOs: 9 and 11-18, and/or light chain sequence of SEQ ID NO: 10.

45. The method of claim 25, wherein the anti-Cx43 antibody comprises heavy chain sequence of SEQ ID NO: 9, and/or light chain sequence of SEQ ID NO: 10.

46. The method of claim 1, wherein the subject is a human.

47. The method of claim 1, wherein the anti-Cx43 antibody blocks the opening of Cx43 hemichannel in the subject.

46. The method of claim 25, wherein the subject is a human.

47. The method of claim 25, wherein the anti-Cx43 antibody blocks the opening of Cx43 in the subject.
